SEQUENCE LISTING (30 sequences)

SEQ ID NO: 1 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 2 - protein, 110 aa, Homo sapiens
MAKHHPDLIFCRKQAGVAIGRLCEKCDGKCVICDSYVRPCTLVRICDECNYGSYQGRCVICGGPGVSDAYYCKECTIQEKDRDGCPKIVNLGSSKTDLFYERKKYGFKKR

SEQ ID NO: 3 - DNA, 344 bp, Artificial Sequence
  source: /note=“Description of Artificial Sequence Synthetic polynucleotide”
actctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagctttgcacgtctagggcgcagtagtccagggtttccttgatgatgtcatactaatcctgtcccttttttttccacagctcgcggttgaggacaaactcttcgcggtctttccagtactcttggatcggaaacccgtcggcctccgaacg

SEQ ID NO: 4 - DNA, 20 bp, Artificial Sequence
  source: /note=“Description of Artificial Sequence Synthetic primer”
actctcttccgcatcgctgt

SEQ ID NO: 5 - DNA, 20 bp, Artificial Sequence
  source: /note=“Description of Artificial Sequence Synthetic primer”
ccgacgggtttccgatccaa

SEQ ID NO: 6 - DNA, 18 bp, Artificial Sequence
  source: /note=“Description of Artificial Sequence Synthetic probe”
ctgttgggctcgcggttg

SEQ ID NO: 7 - DNA, 20 bp, Artificial Sequence
  source: /note=“Description of Artificial Sequence Synthetic primer”
tggcatcagattgcaaagac

SEQ ID NO: 8 - DNA, 20 bp, Artificial Sequence
  source: /note=“Description of Artificial Sequence Synthetic primer”
acgccgggtgatgtatctat

SEQ ID NO: 9 - DNA, 20 bp, Artificial Sequence
  source: /note=“Description of Artificial Sequence Synthetic probe”
cgaaacgcacccgtcagacg

SEQ ID NO: 10 - DNA, 21 bp, Artificial Sequence
  source: /note=“Description of Artificial Sequence Synthetic primer”
atatgccaaaccagctcctac

SEQ ID NO: 11 - DNA, 24 bp, Artificial Sequence
  source: /note=“Description of Artificial Sequence Synthetic probe”
agaactccacaaacccatcccagc

SEQ ID NO: 12 - DNA, 19 bp, Artificial Sequence
  source: /note=“Description of Artificial Sequence Synthetic primer”
aaggacaaaacgggactgg

SEQ ID NO: 13 - DNA, 17 bp, Artificial Sequence
  source: /note=“Description of Artificial Sequence Synthetic primer”
aaagccaatgggcaggt

SEQ ID NO: 14 - DNA, 24 bp, Artificial Sequence
  source: /note=“Description of Artificial Sequence Synthetic probe”
tccacaaacccatcttggaaggcc

SEQ ID NO: 15 - DNA, 22 bp, Artificial Sequence
  source: /note=“Description of Artificial Sequence Synthetic primer”
ccaccttctaggtcctctacat

SEQ ID NO: 16 - DNA, 20 bp, Artificial Sequence
  source: /note=“Description of Artificial Sequence Synthetic primer”
gacaaaggaggccgtgagga

SEQ ID NO: 17 - DNA, 22 bp, Artificial Sequence
  source: /note=“Description of Artificial Sequence Synthetic probe”
gtttgttacgccgtcgctgaaa

SEQ ID NO: 18 - DNA, 23 bp, Artificial Sequence
  source: /note=“Description of Artificial Sequence Synthetic primer”
tcaggcatgcttcggaaactgga

SEQ ID NO: 19 - DNA, 29 bp, Artificial Sequence
  source: /note=“Description of Artificial Sequence Synthetic primer”
gccccggggtgaataataattggtttact

SEQ ID NO: 20 - DNA, 24 bp, Artificial Sequence
  source: /note=“Description of Artificial Sequence Synthetic probe”
tttctaggatgggtttgtggagtt

SEQ ID NO: 21 - DNA, 27 bp, Artificial Sequence
  source: /note=“Description of Artificial Sequence Synthetic primer”
cctgatgccaccttctaggtcctctac

SEQ ID NO: 22 - DNA, 20 bp, Artificial Sequence
  source: /note=“Description of Artificial Sequence Synthetic primer”
gccaaggacacaaagccaat

SEQ ID NO: 23 - DNA, 24 bp, Artificial Sequence
  source: /note=“Description of Artificial Sequence Synthetic probe”
ctggagaccttacgacgggttggg

SEQ ID NO: 24 - DNA, 17 bp, Artificial Sequence
  source: /note=“Description of Artificial Sequence Synthetic primer”
aaggccgtctcgtggtt

SEQ ID NO: 25 - DNA, 3914 bp, Homo sapiens
atggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaaaacccctgatcctaaaatgaatgctaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggccgagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataa

SEQ ID NO: 26 - DNA, 333 bp, Homo sapiens
atggctaaacatcatcctgatttgatcttttgccgcaagcaggctggtgttgccatcggaagactgtgtgaaaaatgtgatggcaagtgtgtgatttgtgactcctatgtgcgtccctgcactctggtgcgcatatgtgatgagtgtaactatggatcttaccaggggcgctgtgtgatctgtggaggacctggggtctctgatgcctattattgtaaggagtgcaccatccaggagaaggacagagatggctgcccaaagattgtcaatctggggagctctaagacagacctcttctatgaacgcaaaaaatacggcttcaagaagaggtga

SEQ ID NO: 27 - protein, 76 aa, Saccharomyces cerevisiae
YAPPKEWMRICFELLELLKSTNKEIRRSANATFGFIAEAIGPHDVLVALLNNLKVQERQLRVCTAVAIGIVAKVCG

SEQ ID NO: 28 - protein, 76 aa, Homo sapiens
YVSAREWMRICFELLELLKAHKKAIRRATVNTFGYIAKAIGPHDVLATLLNNLKVQERQNRVCTTVAIAIVAETCS

SEQ ID NO: 29 - protein, 110 aa, Homo sapiens; identical to SEQ ID NO: 2

SEQ ID NO: 30 - protein, 107 aa, Saccharomyces cerevisiae
MSRHQFDLIMCLKQPGVQTGLLCEKCDGKCPICDSYVRPKRKVRVCENCSFGKQAKNCIICNLNVGVNDAFYCWECCRLGKDKDGCPRILNLGSNRLDRHFEKKKKV

(19) United States
(12) Patent Application Publication
(10) Pub. No.: US 2020/0190593 A1
(43) Pub. Date: Jun. 18, 2020

(54) SPLICEOSOME MUTATIONS AND USES THEREOF

(71) Applicant: EISAI R&D MANAGEMENT CO., LTD., Tokyo (JP)

(72) Inventors: Markus WARMUTH, Newton, MA (US); Xiaoling PUYANG, Stoughton, MA (US); Teng TENG, Winchester, MA (US); Ping ZHU, Acton, MA (US)

(21) Appl. No.: 16/494,202

(22) PCT Filed: Mar. 14, 2018
(86) PCT No.: PCT/US2018/022437

§ 371 Date: Sep. 13, 2019

Related U.S. Application Data
(60) Provisional application No. 62/471,903, filed on Mar. 15, 2017.

(51) Int. Cl. C12Q 1/6886; A61K 31/351; A61K 31/357; A61K 31/365; A61K 31/69

(52) U.S. Cl. CPC C12Q 1/6886; A61K 31/351; A61K 31/357; C12Q 2600/156; A61K 31/69; C12Q 2600/106; A61K 31/365

(57) ABSTRACT

Spliceosome mutations are described herein, including mutations in the PHF5A and SF3B1 subunits. This application also describes detecting the presence and/or absence of mutations in the spliceosome, as well as methods of diagnosing responsiveness to splice modulator treatment, methods of treating neoplastic disorders, and methods of monitoring or altering the treatment based on mutation status.

The present application is a national stage application under 35 U.S.C. § 371 of PCT/US2018/022437, filed Mar. 14, 2018, which designated the U.S. and claims the benefit of priority of U.S. Provisional Application No. 62/471,903, filed Mar. 15, 2017. The entire contents of the foregoing applications are incorporated herein by reference.

### SEQUENCE LISTING

The instant application contains a Sequence Listing which has been submitted electronically in ASCII format and is hereby incorporated by reference in its entirety. Said ASCII copy, created on May 4, 2018, is named 12636_0007-00304_SL.txt and is 27,564 bytes in size.

The present disclosure provides methods for diagnosing, predicting, monitoring, and treating a subject having a neoplastic disorder. Specifically, the methods disclosed herein involve detecting the presence and/or absence of a spliceosome mutation, e.g., a PHF5A mutation, in a subject with a neoplastic disorder and methods for selecting an appropriate treatment regime thereby. Also described herein are methods for treating a subject who has a neoplastic disorder based on their mutation status, as well as methods of monitoring treatment efficacy based on mutation status.

RNA splicing is catalyzed by the spliceosome, a dynamic multiprotein-RNA complex composed of five small nuclear RNAs (snRNAs U1, U2, U4, U5, and U6) and associated proteins. The spliceosome assembles on pre-mRNAs to establish a dynamic cascade of multiple RNA and protein interactions that catalyze excision of the introns and ligation of exons (Matera and Wang, Nature reviews. Molecular cell biology 15, 108-21 (2014)). Accumulating evidence has linked human diseases to dysregulation in RNA splicing that impact many genes (Scotti and Swanson, Nature reviews. Genetics 17, 19-32 (2016)).

The multiprotein-RNA complex of the spliceosome includes, in addition to the five snRNAs, a range of protein subunits such as the SF1-SF3 complexes, U2AF1, and SRSF2. One such unit, the splicing factor SF3b, is itself a multiprotein complex including subunits such as SF3B1, SF3B3, and PHF5A. The SF3b complex is part of the U2 snRNA-protein complex (snRNP) assembled by U2 snRNA, splicing factors SF3a and SF3b, and other associated proteins. Together, these form the 17S U2 snRNP that assembles in an ATP-dependent fashion at the 3′ side of the intron to form the A complex (Bonnal et al., Nature reviews. Drug discovery 11, 847-59 (2012)). The SF3b core complex contains several spliceosome-associated proteins (SAPs), including SF3B1/SAP155, SF3B2/SAP145, SF3B3/SAP130, SF3B4/SAP49, SF3B5/SAP10, SF3B6/SAP14a, and PHF5A/SAP14b.

Recent studies have implicated splicing factors such as SF3B1, U2AF1, and SRSF2 in hematological malignancies including chronic lymphocytic leukemia and myelodysplastic syndromes (Bonnal et al., Nature reviews. Drug discovery 11, 847-59 (2012)). Therefore, recent effort has been devoted to developing splice-modulating small molecules or oligonucleotides as therapeutic approaches to treating these diseases. Some of these have been or are being tested in clinical trials for cancer and severe neuromuscular diseases (Eskens et al., Clinical Cancer Research 19, 6296-304 (2013); Hong et al., Investigational new drugs 32, 436-44 (2014); Naryshkin et al., Science 345, 688-93 (2014); Palacino et al., Nature chemical biology 11, 511-7 (2015)). Nevertheless, patient responsiveness to these splice modulating agents has been inconsistent. Kaida et al., Nature chemical biology 3, 570-5 (2007); Kotake et al., Nature chemical biology 3, 570-5 (2007); Hasegawa et al., ACS chemical biology 6, 229-33 (2011).

Phenotypic resistant clone profiling has been utilized to identify a single amino acid substitution (R1074H) in SF3B1 which almost completely abolishes the splicing-modulating and anti-proliferative effects of pladienolide B and E7107 (Yokoi et al., The FEBS journal 278, 4870-80 (2011)). However, the precise mechanism of inhibition and the role of other components of the SF3b complex remain unclear. Understanding the function and the molecular mechanism of the SF3b complex and its components may help guide the development of next generation spliceosome inhibitors and to allow for targeted treatment to patients who are more likely to respond to splice modulating compounds or to other oncologic intervention strategies.

Accordingly, the present disclosure provides in part, novel approaches to detect, diagnose, prognosticate, treat, and monitor treatment efficacy in patients based on specific spliceosome mutations, particularly in PHF5A and/or SF3B1 that confer resistance to splicing modulation. In addition, methods for treating and identifying a neoplastic disorder are disclosed herein using the mutation status.

In various embodiments, methods of treating a subject having a neoplastic disorder or suspected of having a neoplastic disorder are provided. In some embodiments, the method comprises detecting the presence or absence of a mutation in PHF5A in the subject. In some embodiments, the method also comprises detecting the presence or absence of a mutation in SF3B1 in the subject. In some embodiments, the method comprises administering a splicing modulator to the subject lacking a mutation in PHF5A and/or SF3B1. In some embodiments, the method comprises detecting the presence of a mutation in PHF5A and/or SF3B1 in the subject and administering an alternate therapy that does not target the spliceosome. In some embodiments, the method may comprise obtaining a biological sample from the subject.

In various embodiments, methods of identifying a subject having or suspected of having a neoplastic disorder resistant or responsive to a splicing modulator are provided. In some embodiments, the method comprises obtaining a sample from the subject, and detecting the presence or absence of a mutation in PHF5A. In some embodiments, the method also comprises obtaining a sample from the subject and detecting the presence or absence of a mutation in SF3B1. In some embodiments, the patient is identified as having a treatment-resistant neoplastic disorder when a mutation in PHF5A and/or SF3B1 is detected in the sample. In some embodiments, the patient is identified as having a treatment-responsive neoplastic disorder when a mutation in PHF5A and/or SF3B1 is not detected in the sample.

In various embodiments, methods of determining a treatment regimen for a subject having or suspected of having a neoplastic disorder are provided. In some embodiments, the method comprises identifying the presence or absence of a mutation in PHF5A and/or SF3B1. In some embodiments, the subject is treated with a splicing modulator when a mutation is absent. In some embodiments, the subject is treated with an alternate treatment not targeting the spliceosome when a mutation is present. In some embodiments, the method may comprise obtaining a biological sample from the subject.

In various embodiments, methods of identifying a subject having or suspected of having a neoplastic disorder suitable for treatment with a splicing modulator are provided. In some embodiments, the method comprises obtaining a sample from the subject, and detecting the presence or absence of a mutation in PHF5A and/or SF3B1. In some embodiments, a subject is identified as being suitable for treatment with a splicing modulator when a PHF5A and/or SF3B1 mutation is absent. In some embodiments, provided herein are methods of identifying a subject having or suspected of having a neoplastic disorder suitable for treatment with a splicing modulator, comprising, obtaining a sample from the subject, detecting the presence or absence of a mutation in PHF5A and/or SF3B1, and identifying the subject as suitable for treatment with the splicing modulator when a mutation is absent.

In various embodiments, methods of monitoring splicing modulator treatment efficacy in a subject having or suspected of having a neoplastic disorder are provided. In some embodiments the method comprises administering a splicing modulator to the subject, detecting the presence or absence of a mutation in PHF5A and/or SF3B1 after administering the splicing modulator, and administering a further dose of the splicing modulator if a mutation is absent. In some embodiments, the method can be repeated until a mutation in PHF5A and/or SF3B1 is detected.

In various embodiments, provided herein are methods of detecting a mutation in PHF5A and/or SF3B1 in a subject having or suspected of having a neoplastic disorder. In some embodiments, the method comprises obtaining a tumor sample from the subject, contacting the sample with a splicing modulator, and measuring the growth or volume of the tumor after contact with the splicing modulator.

In various embodiments, the methods provided herein can further comprise administering a splicing modulator to a subject lacking a mutation. In various embodiments, the subject lacking a mutation can be administered herboxidiene, pladienolide, spliceostatin, sudemycin, or a derivative or combination thereof. In some embodiments, the subject is administered spliceostatin A. In some embodiments, the subject is administered sudemycin D.

In some embodiments, the splicing modulator comprises a SF3b complex modulator. In some embodiments the splicing modulator comprises a SF3B1 modulator. In some embodiments, the splicing modulator comprises a PHF5A modulator. In some embodiments, the SF3b modulator is a pladienolide or derivative. In some embodiments the pladienolide or derivative comprises E7107, pladienolide B, or pladienolide D. In some embodiments, the SF3b modulator is a herboxidiene or derivative. In some embodiments, the SF3b modulator is a spliceostatin or derivative. In some embodiments, the spliceostatin comprises FR901464, or spliceostatin A. In some embodiments, the SF3b modulator is a sudemycin or derivative. In some embodiments, the sudemycin comprises sudemycin D6.

In various embodiments, the methods provided herein can comprise administering an alternative treatment that does not target the spliceosome. In some embodiments, the treatment can comprise a cytotoxic agent, a cytostatic agent, or a proteasome inhibitor. In some embodiments, the alternative treatment is a proteasome inhibitor. In some embodiments, the proteasome inhibitor is bortezomib.

In various embodiments, the PHF5A mutation is located in or near the PHF5A-SF3B1 interface. In some embodiments, the mutation in or near the PHF5A-SF3B1 interface is a mutation at position Y36 in PHF5A, and/or one or more mutations at a position selected from K1071, R1074, and V1078 in SF3B1. In some embodiments, the PHF5A mutation comprises a Y36C mutation, or a Y36A, Y36C, Y36S, Y36F, Y36W, Y36E, or Y36R mutation. In some embodiments, the PHF5A mutation comprises a Y36C mutation. In some embodiments, the mutation(s) in SF3B1 comprise one or more of a K1071E mutation, an R1074H mutation, and/or a V1078A or V1078I mutation. In some embodiments, a Y36 mutation in PHF5A and/or a K1071, R1074, and V1078 mutation in SF3B1 indicates that the subject is resistant to treatment with a herboxidiene, pladienolide, spliceostatin, or sudemycin, or a derivative or combination thereof. In some embodiments, the lack of a mutation indicates that the subject may be responsive to treatment with a herboxidiene, pladienolide, spliceostatin, or sudemycin, or a derivative or combination thereof.

In some embodiments, the method may further comprise determining whether the subject has a neoplastic disorder by identifying an SF3B1 mutation selected from one or more of E622D, E622K, E622Q, E622V, Y623C, Y623H, Y623S, R625C, R625G, R625H, R625L, R625P, R625S, R1074H, N626D, N626H, N626I, N626S, N626Y, H662D, H662L, H662Q, H662R, H662Y, T663I, T663P, K666E, K666M, K666N, K666Q, K666R, K666S, K666T, K700E, V701A, V701F, V701I, I704F, I704N, I704S, I704V, G740E, G740K, G740R, G740V, K741N, K741Q, K741T, G742D, D781E, D781G, and D781N.

In various embodiments, the neoplastic disorder may be a hematological malignancy, solid tumor, or a soft tissue sarcoma. In some embodiments, the neoplastic disorder is a hematological malignancy. In some embodiments, the hematological malignancy is myelodysplastic syndrome, chronic lymphocytic leukemia, chronic myelomonocytic leukemia, or acute myeloid leukemia.

In some embodiments, the methods provided herein comprise obtaining a sample from the subject. In some embodiments, the sample can be from blood, a blood fraction, or a cell obtained from the blood or blood fraction. In some embodiments, the sample can be solid tumor sample.

In various embodiments, the methods provided herein comprise detecting the presence or absence of a mutation by comparing to a wild-type protein or nucleic acid sequence of PHF5A and/or SF3B1. In some embodiments, determining or identifying a mutation sequencing a nucleic acid, e.g., using one or more of PCR amplification, in situ PCR in a sample, Sanger sequencing, whole exome sequencing, single nucleotide polymorphism analysis, deep sequencing, targeted gene sequencing, or any combination thereof. In some embodiments, the sequencing comprises PCR amplification, real time-PCR, or targeted gene sequencing of the PHF5A and/or SF3B1 genes.

In various embodiments kits are provided, comprising a reagent that detects a mutation in PHF5A and/or SF3B1. In some embodiments, the kit may further include instructions for use to detect a mutation.

### BRIEF DESCRIPTION OF THE SEQUENCE LISTING

SEQ ID NO 1: amino acid sequence of human SF3B1 protein.

SEQ ID NO 2: amino acid sequence of human PHF5A protein.

SEQ ID NO 3: Ad2-derived nucleic acid sequence.

SEQ ID NO 4: Ad2 forward primer.

SEQ ID NO 5: Ad2 reverse primer.

SEQ ID NO 6: Ad2 reverse probe.

SEQ ID NO 7: Ftz forward primer

SEQ ID NO 8: Ftz reverse primer

SEQ ID NO 9: Ftz probe

SEQ ID NO 10: MCL1-L forward primer

SEQ ID NO 11: MCL1-L probe

SEQ ID NO 12: MCL1-L reverse primer

SEQ ID NO 13: MCL1-S forward primer

SEQ ID NO 14: MCL1-S probe

SEQ ID NO 15: MCL1-S reverse primer

SEQ ID NO 16: MCL1 intron1 forward primer

SEQ ID NO 17: MCL1 intron1 probe

SEQ ID NO 18: MCL1 intron1 reverse primer

SEQ ID NO 19: MCL1 intron2 forward primer

SEQ ID NO 20: MCL1 intron2 probe

SEQ ID NO 21: MCL1 intron2 reverse primer

SEQ ID NO 22: pan MCL1 forward primer

SEQ ID NO 23: pan MCL1 probe

SEQ ID NO 24: pan MCL1 reverse primer

SEQ ID NO 25: nucleic acid sequence of human SF3B1 protein.

SEQ ID NO 26: nucleic acid sequence of human PHF5A protein.

### BRIEF DESCRIPTION OF THE DRAWINGS

The drawings are not necessarily to scale or exhaustive. Instead, emphasis is generally placed upon illustrating the principles of the inventions described herein. The accompanying drawings, which constitute a part of this specification, illustrate several embodiments consistent with the disclosure and, together with the description, serve to explain the principles of the disclosure. In the drawings:

FIG. 1A depicts E7107 and herboxidiene resistant clone generation and whole exome sequencing (WXS) analysis. FIG. 1B depicts recurrent mutations in E7107 and herboxidiene resistant clones. FIGS. 1C-1G show 72 hour growth inhibition profiling (CellTiter-Glo cellular viability assay) of representative resistant clones response to indicated compounds. Error bar indicates standard deviation. For E7107, herboxidiene and bortezomib, n=4; for spliceostatin A and sudemycin D6, n=2.

FIG. 2A shows a Western blot analysis of PHF5A levels in parental, PHF5A WT expressing and PHF5A Y36C expressing HCT116 cells. GAPDH is shown as a loading control. FIG. 2B shows proliferation of parental, WT PHF5A expressing or Y36C PHF5A expressing HCT116 cells as measured by Incucyte imaging system. X-axis indicates hours post seeding, y-axis indicates percent of confluency. Error bar indicates standard deviation, n=5. FIG. 2C shows a Western blot analysis of indicated SF3b complex protein levels following anti-SF3B1 pull-down from nuclear extracts containing WT or Y36C PHF5A. FIG. 2D depicts 72 hr growth inhibition profiling (CellTiter-Glo cellular viability assay) of parental, PHF5A WT expressing and PHF5A Y36C expressing HCT116 cells in response to indicated splicing modulators. Error bar indicates standard deviation, n=2.

FIG. 3A depicts an in vitro splicing assay in the presence of indicated splicing modulators in nuclear extracts containing WT or Y36C PHF5A. Error bar indicates standard deviation, n=4. FIG. 3B shows Taqman gene expression analysis of mature SLC25A19 mRNA levels and EIF4A1 pre-mRNA levels in either WT or Y36C PHF5A expressing cells treated with indicated splicing modulators. All data points were normalized to the corresponding DMSO treated control samples and displayed in logarithmic scale on the y-axis. Error bar indicates standard deviation, n=2.

FIG. 4A shows a stacked bar graph of the counts (left panel) and fractions (right panel) of differential splicing events in each indicated treatment group as compared to DMSO controls. FIG. 4B depicts a summary of the counts and log 2 fold changes of differential splicing events in indicated treatment group as compared to DMSO controls.

FIG. 4C shows plot of average GC content within retained introns and downstream exons from E7107 induced intron-retention junctions. Each intron was normalized to 100 bins whereas each exon to 50 bins. Dark line represents average GC content of each bin; shaded region indicates the 95% confidence interval. FIG. 4D depicts plot of average GC content within skipped-exons and both upstream (left) and downstream (right) introns from E7107 induced exon-skipping junctions. Each intron was normalized to 100 bins whereas each exon to 50 bins (see Methods for details). Dark line represents average GC content of each bin; shaded region indicates the 95% confidence interval. FIG. 4E shows a waterfall plot of the 3′ junction usage of 3883 junctions in E7107 treated PHF5A Y36C (top) and WT (bottom) cells. X-axis on both panels is ordered based on the ES PSI (percentage spliced in) value (large to small) of each junction in E7107 treated Y36C line. On Y-axis the PSI of either exon-skipping (ES) or intron-retention (IR) of the same 3′ junction were shown. The PSI of exon-skipping event, the intron-retention event and exon-inclusion event (not shown in graph) for each junction add up to 100 for each junction. The scheme of PSI calculation is shown below waterfall plots.

FIG. 5A shows a representative sashimi plot of the production of different MCL1 isoforms under indicated treatment from either WT or Y36C PHF5A over-expressing cells. Total reads for each track are shown on the left. FIG. 5B depicts Taqman gene expression analysis of indicated MCL1 isoforms in either WT (left panel) or Y36C (right panel). PHF5A expressing cells treated with splicing modulators. Error bar indicates standard deviation, n=2.

FIG. 6A shows a ribbon diagram of PHF5A (PDB:5SYB). Zinc atoms are shown as gray balls and form the vertices of a near equilateral triangle. The secondary structural elements (α: helix, η: 310 helix, β: strand) forming the sides of the trefoil knot are arranged by their primary sequence. The N and C termini are labeled. Cysteine residues are shown as sticks, as is the Y36 residue. FIG. 6B shows a model of PHF5A in the yeast B^{act} complex. Yeast PHF5A, SF3B5 and SF3B1 formed a complex that made contacts to the RNA duplex base-paired by U2 snRNA and the branch point sequence (BPS), and as well as a single stranded intron RNA at the downstream of BPS. FIG. 6C shows a sequence alignment of the HEAT repeat 15 and 16 where this part of Hsh155 formed adenine binding site with Rds3. FIG. 6C discloses SEQ ID NOS 27-28, respectively, in order of appearance. FIG. 6D shows a sequence alignment of PHF5A with Rds3. The sequence identity is 56%. FIG. 6D discloses SEQ ID NOS 29-30, respectively, in order of appearance. FIG. 6E depicts a potential configuration of human adenine binding site showing interactions between PHF5A, SF3B1 and intron RNA. FIG. 6F shows a surface view of the potential modulator binding site composed by SF3B1, PHF5A and SF3B3. Drug resistant residues are indicated.

FIG. 7A shows coomassie staining of the recombinant four-protein mini-complexes containing PHF5A-WT or PHF5A-Y36C used for Scintillation Proximity Assays. FIG. 7B depicts the competitive titration curves of non-radioactive splicing modulators to ³H-labelled pladienolide analogue (10 nM) binding to the WT four protein complex. FIG. 7C shows the overall surface view of modeled C36 overlaid onto WT (Y36 show in cyan stick) and zoom-in PHF5A surface view at Y36 and C36. Surface potential colored in red: −8 kBT/e, blue: +8 kBT/e and white: 0 kBT/e, was calculated by APBS. FIG. 7D depicts a scintillation proximity assay of the ³H-labelled pladienolide analogue (10 nM and 1 nM) binding to protein complexes containing WT or Y36C PHF5A. Error bar indicates standard deviation, n=2. FIG. 7E shows a Western blot analysis of PHF5A levels in parental and indicated PHF5A variants expressing HCT116 cells. GAPDH is shown as a loading control. FIG. 7F depicts an unsupervised clustering heatmap of the IC50 shift between indicated PHF5A variant expressing cell lines as compared to WT cell lines. The shift is shown as fold changes and calculated from IC50 values extracted from dose response curves in FIG. 7G. Each row represents indicated PHF5A variant and each column corresponds to indicated compound. Color key is shown on the top right corner of FIG. 7G. 72 hr growth inhibition profiling (CellTiter-Glo cellular viability assay) of parental and indicated PHF5A variant expressing HCT116 cells' response to indicated compounds. Error bar indicates standard deviation, n=3.

FIG. 8 depicts the molecular surface representation of the protein complex SF3B1, PHF5A, and SF3B3. The intron RNA and branch point adenosine (BPA) are labelled. The common splicing modulators binding site is indicated by a star with the approximate positions of the surrounding residues for which resistance mutations were identified. The figure was generated using the yeast B^{act} complex coordinates. The schematic model indicates the inverse correlation between the GC content of the intron sequence and their resistance to splicing modulation. Specifically, high GC content intron substrates are weaker substrates that show more sensitivity or less resistance to splicing modulators.

FIG. 9 is a graph showing the G150 shift in PHF5A Y36C and R1074H clones. X-axis is the G150 ratios between the PHF5A Y36C mutation carrying clone versus the parental line of the same compound in logarithm scale. Y-axis is the G150 ratios between the SF3B1 R1074H mutation carrying clone versus the parental line in logarithm scale. The line at 45° diagonal represents equal GI50 shift of the same compound in both resistant clones as compared to the parental line.

FIG. 10 is a graph showing that PHF5A Y36C over-expression in PANC0504 cells yields a partial resistant phenotype to splicing modulator E7107 but not proteasome inhibitor bortezomib.

FIG. 11A shows a Scintillation Proximity Assay (SPA). FIG. 11B is a graph of the Scintillation Proximity Assay for the 3H-labelled pladienolide analogue (10 nM) binding to anti-SF3B1 or mock immunoprecipitated SF3b complex from nuclear extracts containing WT or Y36C PHF5A. Pre-treatment of unlabeled compounds (10 μM) were included when indicated.

### DETAILED DESCRIPTION OF ILLUSTRATIVE EMBODIMENTS

Provided herein are exemplary detailed descriptions of the disclosure. The embodiments within the specification should not be construed to limit the scope of the disclosure.

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The terms “subject” and “patient” are used interchangeably herein to refer to any animal, such as any mammal, including but not limited to, humans, non-human primates, rodents, and the like. In some embodiments, the mammal is a mouse. In some embodiments, the mammal is a human.

The terms “neoplastic disorder” and “cancer” are used herein interchangeably to refer to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The terms “neoplastic disorder” and “cancer” includes all types of cancers and cancer metastases, including hematological malignancy, solid tumors, sarcomas, carcinomas and other solid and non-solid tumor cancers.

The terms “effective amount” and “therapeutically effective amount” as used herein refer to that amount of a compound described herein (e.g., a splicing modulator or an alternative treatment) that is sufficient to effect the intended result including, but not limited to, disease treatment, as illustrated below. In some embodiments, the “therapeutically effective amount” is the amount that is effective for detectable killing, reduction, and/or inhibition of the growth or spread of tumor cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, the subject and their age and existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treat”, “treatment” or “treating” and grammatically related terms, refer to any improvement of any sign, symptom, or consequence of disease, such as prolonged survival, less morbidity, and/or a lessening of side effects which are the byproducts of an alternative therapeutic modality such as tumor cell growth, cancer cell proliferation, and/or metastasis. As is readily appreciated in the art, full eradication of disease is preferred but not a requirement for treatment. In various embodiments, “treatment” or “treat,” as used herein, refer to the administration of a splicing modulator or an alternative treatment to a subject having a neoplastic disorder, e.g., a patient. The treatment can be to cure, heal, alleviate, relieve, reduce, alter, remedy, ameliorate, palliate, improve or affect the disorder, the symptoms of the disorder, or the predisposition toward the disorder, e.g., a neoplastic disorder.

As used herein, the terms “splice modulator” or “splicing modulator” refer to compounds that have anti-tumor activity by interacting with components of the spliceosome. In some embodiments, a splicing modulator alters the rate or form of splicing in a target cell. Splicing modulators that function as inhibitory agents, for example, are capable of decreasing uncontrolled cellular proliferation. In particular, in some embodiments the splicing modulators may act by inhibiting the SF3b subunit of the spliceosome, e.g., by targeting the ST3B1 and/or PHF5A subunits. Such modulators may be natural compounds or synthetic compounds. Non-limiting examples of splicing modulators and categories of such modulators include pladienolide, pladienolide derivatives, herboxidiene, herboxidiene derivatives, spliceostatin, spliceostatin derivatives, sudemycin, or sudemycin derivatives. As used herein, the terms “derivative” and “analog” when referring to a splicing modulator, or the like, means any such compound that retains essentially the same, similar, or enhanced biological function or activity as the original compound but an altered chemical or biologic structure.

As used herein, a “spliceosome” refers to a ribonucleoprotein complex that removes introits from one or more RNA segments, such as pre-mRNA segments.

As used herein, the term “treatment resistant neoplastic disorder” refers to a neoplastic disorder (i.e., a cancer) that does not respond to a splicing modulator.

The term “detecting” includes determining the presence or absence of a mutation in the SF3b complex, e.g., in PHF5A and/or SF3B1. Additionally, “evaluating” includes distinguishing patients that may be successfully treated with a splicing modulator from those who will not.

### A. SF3B1 and PHF5A and Mutations Therein

The present disclosure relates, in part, to mutations affecting genes encoding components of the spliceosome that result in defective splicing. In various embodiments, the mutation is in the PHF5A subunit. In various embodiments, a mutation is in the SF3B1 subunit. The presence of a mutation in the spliceosome can be indicative of a subject's responsiveness or lack thereof to a splicing modulator. For example, a subject harboring particular PHF5A gene mutations can have decreased sensitivity to splicing modulators.

Two unique spliceosomes coexist in most eukaryotes: the U2-dependent spliceosome, which catalyzes the removal of U2-type introns, and the less abundant U12-dependent spliceosome, which is present in only a subset of eukaryotes and splices the rare U12-type class of introns. The independent spliceosome is assembled from the U1, U2, U5, and U4/U6 snRNPs and numerous non-snRNP proteins. The U2 snRNP is recruited with two weakly bound protein subunits, SF3a and SF3b, during the first ATP-dependent step in spliceosome assembly. SF3b is composed of seven conserved proteins; including PHF5A, SF3M55, SF3M45, SF3b130, SF3b49, SF3b14a, and SF3M0 (Will et al., EMBO J. 27, 4978, 2002).

PHD finger-like domain-containing protein 5A (also referred to as PHF5A) contains a Plant Homeo Domain (PHD)-finger-like domain that is flanked by highly basic amino- and carboxy-termini; therefore, PHF5A belongs to the PHD-finger superfamily but it may also act as a chromatin-associated protein. The PHF5A protein bridges the U2 snRNP with the U2AF1 (a U2AF65-U2AF35 heterodimer) associated with the 3′-end of the intron and RNA helicase DDX1 (Rzymski et al., Cytogenet. Genome Res. 121, 232, 2008). Stable U2 snRNP addition is often a regulated step in alternative pre-mRNA splicing. In certain embodiments, the wild-type human PHF5A protein is as set forth in SEQ ID NO: 2 (GenBank Accession Number NP_032758, Version NP_032758.3. In certain embodiments, mutations in PHF5A are identified by differing from the amino acid sequence of the human wild type PHF5A protein provided in SEQ ID NO: 2, or an encoding nucleic acid as set further in SEQ ID NO: 26 (GenBank Accession NM_032758 Version NM_032758.3), and by a resulting cancer phenotype (i.e., they are not natural allelic variants that do not correlate with cancer in a subject).

SF3B1 is a component of the spliceosome and forms part of the U2 snRNP complex which binds to the pre-mRNA at a region containing the branchpoint site and is involved in early recognition and stabilization of the spliceosome at the 3′ splice site (3′ss). In certain embodiments, the wild-type human SF3B1 protein is as set forth in SEQ ID NO: 1 (GenBank Accession Number NP_036565, Version NP_036565.2) (Bonnal et al., Nature Review Drug Discovery 11, 847-59 (2012)) or SEQ ID NO: 25 (GenBank Accession Number NM_012433, Version NM_012433.3). Mutations in genes encoding the SF3B3 protein are implicated in a number of cancers, such as hematologic malignancies and solid tumors (Scott et al., JNCI 105, 20, 1540-1549 (2013). In certain embodiments, mutations in SF3B1 are identified by differing from the amino acid sequence of the human wild type SF3B1 protein provided in SEQ ID NO: 1, or an encoding nucleic acid as set forth in SEQ ID NO: 25, and by a resulting cancer phenotype (i.e., they are not natural allelic variants that do not correlate with cancer in a subject).

In some embodiments, a subject has a tumor or cancer cell harboring one or more PHF5A mutations and/or one or more SF3B1 mutations, or a subject is tested for the presence or absence of such mutation(s).

In some embodiments, the one or more PHF5A and/or SF3B1 mutations can include a point mutation (e.g., a missense or nonsense mutation), an insertion, and/or a deletion. In other embodiments, the one or more PHF5A and/or SF3B1 mutations can include a somatic mutation. In still other embodiments, the one or more PHF5A and/or SF3B1 mutations can include a heterozygous mutation or a homozygous mutation. In certain embodiments, a PHF5A mutation is present in combination with an SF3B1 mutation. In other embodiments, a PHF5A mutation and/or an SF3B1 mutation is mutually exclusive.

In various embodiments, one or more mutations present in PHF5A and/or SF3B1 are in a tumor or cancer cell from a subject, or the subject is tested for the presence or absence of such mutation(s). In certain embodiments, a PHF5A mutation can be located in or near the PHF5A-SF3B1 interface. In some embodiments, a PHF5A mutation can be located in the PHF5A-SF3B1 interface. In some embodiments the PHF5A mutation can be located near the PHF5A-SF3B1 interface.

In various embodiments, the one or more PHF5A mutations comprise a mutation at position Y36 of PHF5A. In some embodiments, the mutation at position Y36 is the only mutation in PHF5A, while in other embodiments additional mutations are present in PHF5A. In some embodiments, the mutation at position Y36 is accompanied by one or more mutations in SF3B1 (e.g., a mutation at one or more of positions K1071, R1074, and V1078). In some embodiments, the mutation at position Y36 is not accompanied by any mutations in SF3B1.

In various embodiments, the Y36 mutation in PHF5A is selected from a Y36C, Y36A, Y36S, Y36F, Y36W, Y36E, and Y36R mutation. In certain embodiments, the PHF5A mutation is Y36C.

In certain embodiments, the one or more mutations in SF3B1 are selected from one or more of a K1071E mutation, an R1074H mutation, and/or a V1078A or V1078I mutation. In various embodiments, additional mutations are present in SF3B1. In certain embodiments, no mutation is present at positions K1071, R1074, and/or V1078. In certain embodiments, alternate mutations are present in SF3B1 outside of positions K1071, R1074, and V1078.

In some embodiments, one or more further mutations are present in SF3B1. In some embodiments, the additional mutation is one or more of an E622D, E622K, E622Q, E622V, Y623C, Y623H, Y623S, R625C, R625G, R625H, R625L, R625P, R625S, N626D, N626H, N626I, N626S, N626Y, H662D, H662L, H662Q, H662R, H662Y, T663I, T663P, K666E, K666M, K666N, K666Q, K666R, K666S, K666T, K700E, V701A, V701F, V701I, I704F, I704N, I704S, I704V, G740E, G740K, G740R, G740V, K741N, K741Q, K741T, G742D, D781E, D781G, or D781N mutation. In some embodiments, these mutations are used to identify a patient who has cancer. In certain embodiments, SF3B1 mutations may include one or more of K700E, K666N, R625C, G742D, R625H, E622D, H662Q, K666T, K666E, K666R, G740E, Y623C, T663I, K741N, N626Y, T663P, H662R, G740V, D781E, or R625L. In some embodiments, a mutation within SF3B1 may include E622D, R625H, H662D, K666E, K700E, G742D, and/or K700E. Additional SF3B1 mutations include, without limitation, those described in, e.g., Papaemmanuil et al., N. Engl. J. Med. 365:1384-1395 (2011) and Furney et al., Cancer Discov., 3(10):1122-1129 (2013).

Spliceosome modulators generally act preferentially on tumor cells in a gene/mutation-specific manner (Fan et al., ACS Chem. Biol. 6, 582-589 (2011)). In various embodiments, a PHF5A mutation and/or an SF3B1 mutation confers resistance to cancer treatment with a splicing modulator. In other embodiments, a mutation in PHF5A and/or SF3B1 results in reduced activity or altered activity of the splicing modulator. In some embodiments, a mutation in PHF5A alone confers resistance to treatment with a splicing modulator. In some embodiments, a mutation in PHF5A and a mutation in SF3B1 confers resistance to treatment with a splicing modulator.

In certain embodiments, a mutation in PHF5A can confer or increase resistance to a pladienolide or pladienolide derivative, a herboxidiene or herboxidiene derivative, a spliceostatin or a spliceostatin derivative, and/or a sudemycin or a sudemycin derivative, as compared to a subject having a cancer lacking that mutation. In some embodiments, a mutation at position Y36 in PHF5A can confer or heighten resistance to a pladienolide or pladienolide derivative, a herboxidiene or herboxidiene derivative, a spliceostatin or a spliceostatin derivative, and a sudemycin or a sudemycin derivative. In certain embodiments, the mutation is a Y36C mutation. In some embodiments, a Y36C mutation in PHF5A can confer or heighten resistance to E7107 FR901464, herboxidiene, pladienolide, spliceostatin A, and/or sudemycin D.

In some embodiments, a Y36C mutation in PHF5A can confer or heighten resistance to E7107. In some embodiments, a Y36C mutation in PHF5A can confer or heighten resistance to herboxidiene. In some embodiments, a Y36C mutation in PHF5A can confer or heighten resistance to FR901464. In some embodiments, a Y36C mutation in PHF5A can confer or heighten resistance to pladienolide. In some embodiments, a Y36C mutation in PHF5A can confer or heighten resistance to spliceostatin A. In some embodiments, a Y36C mutation in PHF5A can confer or heighten resistance to sudemycin D.

In various embodiments, a mutation in PHF5A in combination with one or more mutations in SF3B1 can confer or increase resistance to a pladienolide or pladienolide derivative, a herboxidiene or herboxidiene derivative, a spliceostatin or a spliceostatin derivative, and/or a sudemycin or a sudemycin derivative, as compared to a subject having a cancer lacking that combination of mutations. In certain embodiments, the PHF5A mutation comprises a mutation at position Y36 and the SF3B1 mutation comprises a mutation at one or more of positions K1071, R1074, and V1078. In some embodiments, the mutation at position Y36 is a Y36C, Y36A, Y36S, Y36F, Y36W, Y36E, or Y36R mutation. In some embodiments, the mutation at one or more of positions K1071, R1074, and V1078 comprise a K1071E mutation, an R1074H mutation, and/or a V1078A or V1078I mutation.

In certain embodiments, a cancer in a subject does not have a Y36 mutation in PHF5A but does have one or more mutations in SF3B1 at one or more of positions K1071, R1074, and V1078. In some embodiments, the mutation at one or more of positions K1071, R1074, and V1078 comprise a K1071E mutation, an R1074H mutation, and/or a V1078A or V1078I mutation. In some embodiments, a mutation in SF3B1 can confer or increase resistance to a pladienolide or pladienolide derivative, a herboxidiene or herboxidiene derivative, a spliceostatin or a spliceostatin derivative, and/or a sudemycin or a sudemycin derivative, as compared to a subject having a cancer lacking such a mutation.

### B. Splicing Modulators

A variety of splicing modulator compounds are known in the art (see, e.g., Lee and Abdel-Wahab, Nature Medicine 7, 976-86 (2016)), and can be used in accordance with the methods described herein (e.g., administered to patients having cancers comprising or lacking all or certain mutations in PHF5A and/or SF3B1). For example, bacterially derived products and their analogs have been shown to target the SF3b complex. These compounds may be useful in the treatment of neoplastic disorders. In some embodiments the splicing modulator is an SF3B1 modulator. In some embodiments the splicing modulator is a PHF5A modulator. In some embodiments, combinations of modulators may be used.

In some embodiments, the splice modulating compound is a pladienolide or pladienolide derivative. A “pladienolide derivative” refers to a compound which is structurally related to a member of the family of natural products known as the pladienolides and which retains one or more biological functions of the starting compound. Pladienolides were first identified in the bacteria Streptomyces platensis (Mizui et al., The Journal of Antibiotics. 57, 188-96 (2004)) as being potently cytotoxic and resulting in cell cycle arrest in the G1 and G2/M phases of the cell cycle (e.g., Bonnal et al., Nature Reviews, Drug Discovery 11, 847-59 (2012)). There are seven naturally occurring pladienolides, pladienolide A-G (Mizui et al., The Journal of Antibiotics. 57, 188-96 (2004); Sakai et al., The Journal of Antibiotics, 57, 180-7 (2004)).

One of these compounds, pladienolide B, has been shown to target the SF3b spliceosome to inhibit splicing and alter the pattern of gene expression (Kotake et al., Nature Chemical Biology 3:570-575 (2007)). Certain pladienolide B analogs are described in, e.g., WO 2002/060890; WO 2004/011459; WO 2004/011661; WO 2004/050890; WO 2005/052152; WO 2006/009276; and WO 2008/126918.

U.S. Pat. Nos. 7,884,128 and 7,816,401 describe methods for synthesizing pladienolide B and D. Synthesis of pladienolide B and D may also be performed using methods described in Kanada et al., Angew. Chem. Int. Ed., 46, 4350-4355 (2007); U.S. Pat. No. 7,550,503, and International Publication No. WO 2003/099813 (describes methods for synthesizing E7107 (compound 45; a synthetic urethane derivative of pladienolide B) from pladienolide D (11107D)).

In some embodiments the splice modulating compound is pladienolide B, pladienolide D, or E7107. In some embodiments, the modulating compound is pladienolide B. In other embodiments, the modulating compound is pladienolide D. In further embodiments, the SF3B1 modulator is E7107.

In some embodiments, the splice modulating compound is a pladienolide compound having a structure as set forth below:

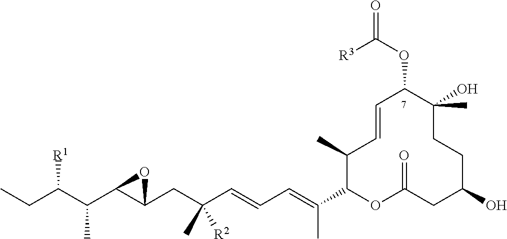

| Compound | R¹ | R² | R³ |
|---|---|---|---|
| pladienolide B | OH | H | Me |
| D | OH | OH | Me |
| E7107 | OH | OH | 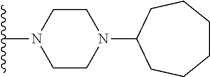 [SMILES: *N1CCN(C2CCCCCC2)CC1] |
| FD-895 | OH | H | Me |

In some embodiments, the splice modulating compound is a compound described in U.S. Publication No. 20150329528. In some embodiments, the modulating compound is a pladienolide compound having any one of formulas 1-4 as set forth in Table 1.

TABLE 1
Pladienolide Compound Structure

| | |
|---|---|
| 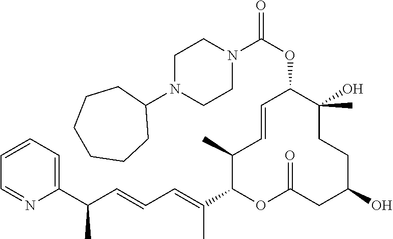 [SMILES: C/C(=C\C=C\[C@@H](C)c1ccccn1)[C@H]1OC(=O)C[C@H](O)CC[C@@](C)(O)[C@@H](OC(=O)N2CCN(C3CCCCCC3)CC2)/C=C/[C@@H]1C] | 1 |
| 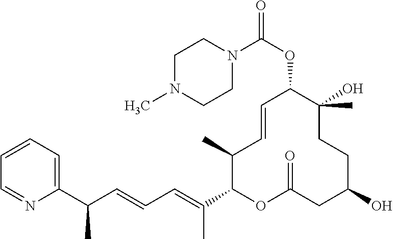 [SMILES: C/C(=C\C=C\[C@@H](C)c1ccccn1)[C@H]1OC(=O)C[C@H](O)CC[C@@](C)(O)[C@@H](OC(=O)N2CCN(C)CC2)/C=C/[C@@H]1C] | 2 |
| 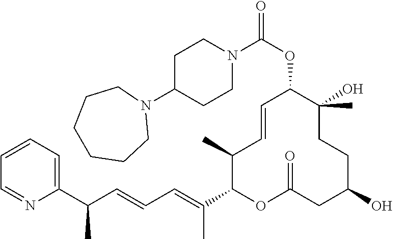 [SMILES: C/C(=C\C=C\[C@@H](C)c1ccccn1)[C@H]1OC(=O)C[C@H](O)CC[C@@](C)(O)[C@@H](OC(=O)N2CCC(N3CCCCCC3)CC2)/C=C/[C@@H]1C] | 3 |
| 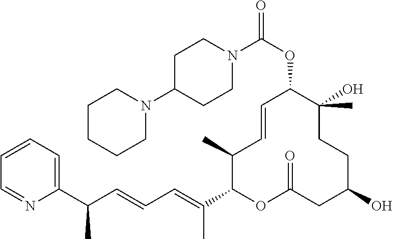 [SMILES: C/C(=C\C=C\[C@@H](C)c1ccccn1)[C@H]1OC(=O)C[C@H](O)CC[C@@](C)(O)[C@@H](OC(=O)N2CCC(N3CCCCC3)CC2)/C=C/[C@@H]1C] | 4 |

In some embodiments, the splice modulating compound may be FD-895. FD-895 is a pladienolide-like member (Kashyap et al., Haematological, 100, 945-954 (2015)). It is derived from Streptomyces hygroscopicus A-9561 (see, e.g., Seki-Asano et al., Journal of Antibiotics, 47, 1395-401 (1994)).

In some embodiments, the splice modulating compound is a FD-895 compound having a structure as set forth below:

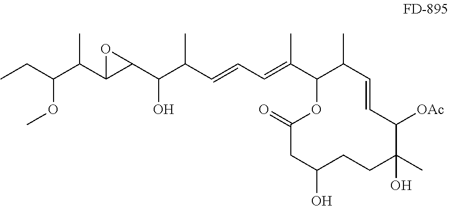 [SMILES: CCC(OC)C(C)C1OC1C(O)C(C)/C=C/C=C(\C)C1OC(=O)CC(O)CCC(C)(O)C(OC(C)=O)/C=C/C1C]

In some embodiments, the splice modulating compound is a herboxidiene or herboxidiene derivative. Herboxidiene is a form of GEX1A. A “herboxidiene derivative” refers to a compound which is structurally related to a member of the herboxidiene or GEX1A family and which retains one or more biological functions of the starting compound. Herboxidiene analogs also include other GEX family members. Herboxidiene was first identified in Streptomyces chromofuscus A7847 (Sakai et al., Journal of Antibiotics (Tokyo), 55, 855-62 (2002); Hasegawa et al., ACS Chemical Biology, 18, 229-33 (2011)). Herboxidiene and derivatives provide antitumor activity by targeting the SF3b complex, for example by interfering with the splicing of pre-mRNA. Id. Synthesis of herboxidiene may be performed using the methods described in Lagisetti et al., ACS Chemical Biology, 9, 643-648 (2014). U.S. Pat. No. 5,719,179 also describes methods for preparing herboxidiene. Other techniques to synthesize herboxidiene or herboxidiene derivatives would be readily recognized by one skilled in the art.

In some embodiments, the splice modulating compound is a herboxidiene compound having a structure as set forth below:

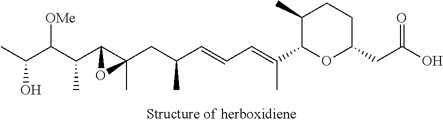 [SMILES: COC([C@@H](C)[C@H]1O[C@]1(C)C[C@H](C)/C=C/C=C(\C)[C@H]1O[C@@H](CC(=O)O)CC[C@@H]1C)[C@@H](C)O]

In other embodiments, the herboxidiene derivative is 6-nor herboxidiene (Lagisetti et al., ACS Chemical Biology, 9, 643-648 (2014)).

In some embodiments, the splice modulating compound is a spliceostatin or spliceostatin derivative. A “spliceostatin derivative” refers to a compound which is structurally related to a member of the family of known spliceostatins and which retains one or more biological functions of the starting compound. Spliceostatins were originally derived from Pseudomonas sp. No. 2663 and are reported to be potent cytotoxic agents targeting SF3b (Lee and Abdel-Wahab, Nature Medicine 7, 976-86 (2016)). U.S. Pat. No. 9,504,669 provides methods for the preparation of spliceostatins and derivatives. Other techniques to synthesize spliceostatins and derivatives would be readily recognized by one skilled in the art.

Exemplary spliceostatin compounds include, but are not limited to, FR901463, FR901464, FR901465, meayamycin, meayamycin B, spliceostatin A (a methylated derivative of FR901464), and thailanstatin. In some embodiments, the splice modulating compound is FR901463. In some embodiments, the splice modulating compound is FR901464. In other embodiments, the splice modulating compound is FR901465. In some embodiments, the splice modulating compound is meayamycin. In another embodiment, the splice modulating compound is meayamycin B. In further embodiments, the splice modulating compound is spliceostatin A.

In some embodiments, the splice modulating compound is a spliceostatin compound having a structure as set forth below:

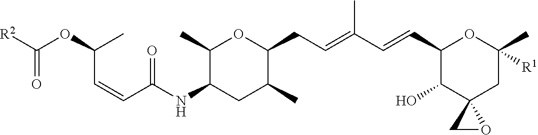

| Compound | R¹ | R² |
|---|---|---|
| FR901464 | OH | Me |
| meayamycin | Me | Me |
| meayamycin B | Me | morpholine |
| spliceostatin A | OMe | Me |

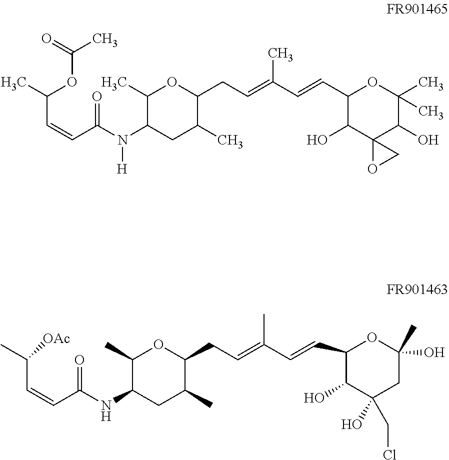 [SMILES: CC(=O)OC(C)/C=C\C(=O)NC1CC(C)C(C/C=C(C)/C=C/C2OC(C)(C)C(O)C3(CO3)C2O)OC1C ; CC(=O)O[C@@H](C)/C=C\C(=O)N[C@@H]1C[C@H](C)[C@H](C/C=C(C)/C=C/[C@H]2O[C@](C)(O)C[C@@](O)(CCl)[C@@H]2O)O[C@@H]1C]

In various embodiments, the splice modulating compound is a thailanstatin or thailanstatin a derivative. A “thailanstatin derivative” refers to a compound which is structurally related to a member of the family of known thailanstatins. Thailanstatins were first identified in Burkholderia thailandensis MSMB43. Three thailanstatins have been isolated from thailanstatin (Liu et al., Journal of Natural Products, 76, 685-93 (2013). In some embodiments, the splice modulating compound is thailanstatin A, thailanstatin B, or thailanstatin C. In other embodiments, the splice modulating compound is thailanstatin A. In some embodiments, the splice modulating compound is thailanstatin B. In some embodiments, the splice modulating compound is thailanstatin C.

In some embodiments, the splice modulating compound is a spliceostatin compound having a structure as set forth below:

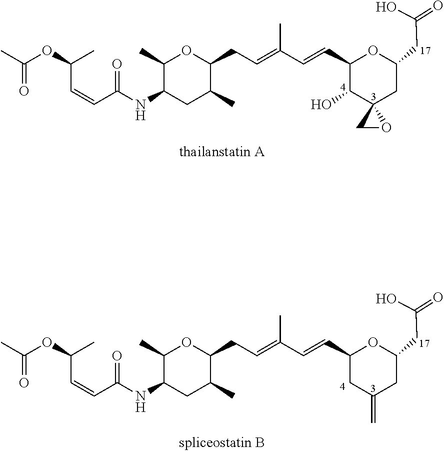 [SMILES: CC(=O)O[C@@H](C)/C=C\C(=O)N[C@@H]1C[C@H](C)[C@H](C/C=C(C)/C=C/[C@H]2O[C@H](CC(=O)O)C[C@@]3(CO3)[C@@H]2O)O[C@@H]1C ; C=C1C[C@@H](CC(=O)O)O[C@H](/C=C/C(C)=C/C[C@@H]2O[C@H](C)[C@H](NC(=O)/C=C\[C@H](C)OC(C)=O)C[C@@H]2C)C1]

In some embodiments, the splice modulating compound is a sudemycin or sudemycin derivative. A “sudemycin derivative” refers to a compound which is structurally related to a member of the family of known sudemycins and which retains one or more biological functions of the starting compound. Sudemycins can be synthesized from derivatives of pladienolide B and FR901464 (see, e.g., Fan et al., ACS Chem. Biol., 6 582-9 (2011)). Sudemycins show the same effects as have been reported for other natural spliceosome modulators including: inhibition of spicing in an in vitro cell-free splicing assay, inhibition of splicing in a cell-based dual reporter assay, cell cycle arrest, and alteration of the cellular localization of SF3b splicing factors. Id. Sudemycins can be synthesized as described by Lagisetti et al., J. Med. Chem., 52, 6979-90, (2009); and Lagisetti et al., J. Med. Chem., 51:6220-24 (2008). Other techniques to synthesize sudemycins and derivatives would be readily recognized by one skilled in the art.

Exemplary splice modulating compounds include, but are not limited to sudemycin C, sudemycin C1, sudemycin D1, sudemycin D6, sudemycin E, and sudemycin F1. In various embodiments, the splice modulating compound is sudemycin C. In certain embodiments, the splice modulating compound is sudemycin C1. In various embodiments, the splice modulating compound is sudemycin D1. In other embodiments, the splice modulating compound is sudemycin D6. In some embodiments, the splice modulating compound is sudemycin E. In other embodiments, the splice modulating compound is sudemycin F1.

In some embodiments, the splice modulating compound is a sudemycin compound having a structure as set forth below:

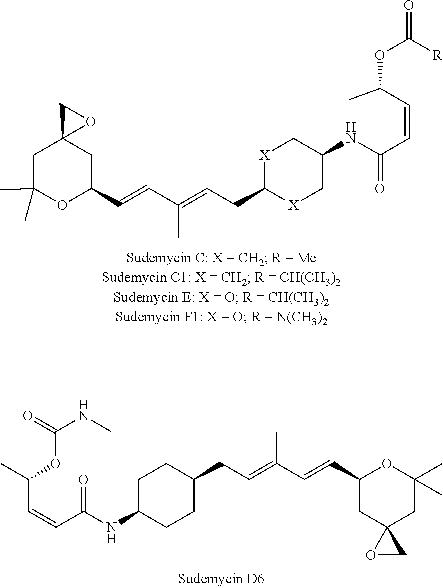

The methods described herein may also be used to evaluate and identify additional known and novel splice modulating compounds, such as compounds targeting the splice complex, for use dependent on PHF5A and/or SF3B1 mutation status. These include alternative derivatives and analogs of herboxidiene, pladienolide, spliceostatin A, and sudemycin.

### C. Sequencing Methods and Samples

Certain embodiments of the methods described herein involve identifying, detecting, and/or determining the presence of a PHF5A mutation and/or a SF3B1 mutation. A variety of methods exists for detecting, quantifying, and sequencing nucleic acids or proteins encoded thereby, and each may be adapted for detection of PHF5A mutations and/or SF3B1 mutations in the embodiments disclosed herein. Exemplary methods include an assay to quantify nucleic acid such as in situ hybridization, microarray, nucleic acid sequencing, PCR-based methods, including real-time PCR (RT-PCR), whole exome sequencing, single nucleotide polymorphism analysis, deep sequencing, targeted gene sequencing, or any combination thereof. In some embodiments, the foregoing techniques and procedures are performed according to methods described in, e.g., Sambrook et al. Molecular Cloning: A Laboratory Manual (3rd ed., Cold Spring Harbor Laboratory Press, Cold Spring Harbor, N.Y. (2000)). See, also, Ausubel et al., Current Protocols in Molecular Biology, ed., Greene Publishing and Wiley-Interscience, New York, 1992 (with periodic updates).

In exemplary PCR-based methods, a particular PHF5A mutation and/or a SF3B1 mutation may be detected by specifically amplifying a sequence that contains or is suspected to contain the mutation. For example, the method may involve obtaining a tumor or cancer cell sample from a patient, isolating genomic DNA, and amplifying the PHF5A and/or SF3B1 gene or a portion thereof surrounding the suspected mutation (e.g., a region including Y36 in PHF5A).

In various embodiments, a PCR-based method may employ a first primer specifically designed to hybridize to a first portion of the PHF5A or SF3B1 gene from a tumor sample. The method may further employ a second opposing primer that hybridizes elsewhere in the PHF5A or SF3B1 gene and/or to a segment of the PCR extension product of the first primer that corresponds to another sequence in the gene, such as a sequence at an upstream or downstream location. In some embodiments, a PCR primer may hybridize to a region containing the suspected mutation (e.g., a region including Y36 in PHF5A) or a region that does not include the suspected mutation position. In various embodiments, the PCR detection method may be quantitative (or real-time) PCR. In some embodiments of quantitative PCR, an amplified PCR product is detected using a nucleic acid probe, wherein the probe may contain one or more detectable labels.

In certain embodiments, sequencing technologies, including but not limited to whole genome sequencing (WGS) and whole exome sequencing (WES), may be used to detect, measure, or analyze a sample for the presence or absence of a PHF5A mutation and/or a SF3B1 mutation. WGS (also known as full genome sequencing, complete genome sequencing, or entire genome sequencing), determines the complete DNA sequence of a subject or cell sample. Exemplary methods for WGS to detect PHF5A mutation and/or SF3B1 mutations in a sample may include those described by Ng and Kirkness, Methods Mol Biol. 628, 215-26 (2010).

WES (also known as exome sequencing, or targeted exome capture) allows for the analysis of many genes, but only exons. Exemplary methods for WES may include those described by Gnirke et al., Nature Biotechnology 27, 182-189 (2009).

In various embodiments, a sample is obtained from a human or non-human animal subject that contains cancer cells or tumor tissue. A “sample” is any biological specimen from a subject. The term includes samples obtained from a variety of biological sources. Exemplary samples include but are not limited to a cell culture, a tissue, a biopsy, oral tissue, gastrointestinal tissue, an organ, an organelle, a biological fluid, a blood sample, a urine sample, a skin sample, and the like. Blood samples may be whole blood, partially purified blood, or a fraction of whole or partially purified blood, such as peripheral blood mononucleated cells (PBMCs). The source of a sample may be a solid tissue sample such as a tumor tissue biopsy. Tissue biopsy samples may be biopsies from, e.g., breast tissue, skin, lung, or lymph nodes. Samples may also be, e.g., samples of bone marrow, including bone marrow aspirate and bone marrow biopsies. Sample may also be liquid biopies, e.g. circulating tumor cells, circulating cell-free tumor DNA, or exosomes.

In certain embodiments, the sample is a human sample. In certain embodiments, the human sample comprises hematological cancer cells or solid tumor cells. Exemplary hematological cancers include chronic lymphocytic leukemia, acute lymphoblastic leukemia, acute myeloid leukemia, chronic myeloid leukemia, chronic myelomonocytic leukemia, acute monocytic leukemia, Hodgkin's lymphoma, Non-Hodgkin's lymphoma, and multiple myeloma. Exemplary solid tumors include carcinomas, such as adenocarcinomas, and may be selected from breast, lung, liver, prostate, pancreatic, colon, colorectal, skin, ovarian, uterine, cervical, or renal cancers. Tumor samples may be obtained directly from a patient or derived from samples obtained from a patient, such as cultured cells derived from a biological fluid or tissue sample. Samples may be archived samples, such as kryopreserved samples, of cells obtained directly from a subject or of cells derived from cells obtained from a patient.

### D. Diagnostic Methods

In various embodiments, provided herein are methods of identifying a subject having or suspected of having a neoplastic disorder suitable for treatment with a splicing modulator. In some embodiments, the methods of identifying a subject having or suspected of having a neoplastic disorder that would benefit from treatment with a splicing modulator may comprise obtaining a biological sample from the subject and detecting the presence or absence of a mutation in PHF5A (either in the protein or in a nucleic acid encoding the protein) alone or in combination with one or more mutations in SF3B1.

In some embodiments, the subject is identified as a suitable candidate for treatment with a splicing modulator in the absence of a PHF5A mutation, particularly in the absence of a mutation at position Y36. In some embodiments, the subject does not have a Y36A, Y36C, Y36S, Y36F, Y36W, Y36E, or Y36R mutation. In some embodiments, the subject does not have a Y36C mutation. The absence of a PHF5A mutation may indicate that the subject is not resistant to treatment with a splicing modulator. The absence of a PHF5A mutation may indicate that the subject may likely benefit from treatment with a splicing modulator. The absence of a PHF5A mutation can also be used to confirm that a tumor initially susceptible to treatment with a splicing modulator has not mutated to become resistant to treatment (e.g., by developing a mutation at position Y36). Thus, in some embodiments, the mutation status of PHF5A can be used to monitor treatment efficacy over the course of treatment, and to determine whether to continue with splice modulator therapy.

In some embodiments, the subject is identified as a suitable candidate for treatment with a splicing modulator in the absence of a mutation in SF3B1. For instance, the subject may be checked for a mutation at one or more of positions K1071, R1074, and V1078 (e.g., a K1071E mutation, an R1074H mutation, and/or a V1078A or V1078I mutation).

In some embodiments, the mutation status of PHF5A and/or SF3B1 can be used to monitor treatment efficacy over the course of treatment, and to determine whether to continue with splice modulator therapy (e.g., by confirming that the subject has not developed a mutation in a cancer cell at position Y36 in PHF5A during treatment).

In other embodiments, the subject is identified as not being a suitable candidate for treatment with a splicing modulator if a cancer sample in the subject contains a PHF5A mutation, particularly a mutation at position Y36, alone or in combination with one or more SF3B1 mutations (e.g., a K1071E mutation, an R1074H mutation, and/or a V1078A or V1078I mutation). In some embodiments, the subject has a Y36A, Y36C, Y36S, Y36F, Y36W, Y36E, or Y36R mutation. In some embodiments, the subject has a Y36C mutation. The presence of a PHF5A and/or SF3B1 mutation may indicate that the subject is resistant to treatment with a splicing modulator, including a herboxidiene, pladienolide, spliceostatin, and sudemycin. The presence of a PHF5A and/or SF3B1 mutation may indicate that the subject is unlikely to benefit from treatment with such a splicing modulator. In some embodiments, the presence of a PHF5A and/or SF3B1 mutation may indicate that the subject is more likely to benefit from an alternative treatment for the cancer that does not target the spliceosome.

A detailed description of methods for treating a subject with a neoplastic disorder is provided in section E (below).

In various embodiments, provided herein are methods of diagnosing a subject with a neoplastic disorder resistant to a splicing modulator by detecting the presence or absence of one or more of the mutations mentioned herein. In certain embodiments, provided herein are methods of diagnosing a subject as having a neoplastic disorder responsive to a splicing modulator by detecting the absence of one or more of the mutations mentioned herein. In some embodiments, diagnosis includes obtaining a biological sample from the subject and detecting the presence or absence of a PHF5A mutation, alone or in combination with an SF3B1 mutation. In some embodiments, the PHF5A mutation is a Y36 mutation. In some embodiments, the presence of a PHF5A mutation results in a diagnosis that the subject has a neoplastic disorder resistant to a splicing modulator. In other embodiments, the absence of a PHF5A mutation results in a diagnosis that the subject has a neoplastic disorder responsive to a splicing modulator. In some embodiments, the SF3B1 mutation comprises a mutation at one or more of positions K1071, R1074, and V1078 (e.g., a K1071E mutation, an R1074H mutation, and/or a V1078A or V1078I mutation).

In certain embodiments, provided herein are methods for detecting a mutation in PHF5A and/or SF3B1 in a subject having or suspected of having a neoplastic disorder. Such methods may include obtaining a tumor sample from a subject, contacting the tumor sample with a splicing modulator, and measuring the growth, volume, or size of the tumor after contact with the splicing modulator. In some embodiments, a decrease in the growth, volume, or size of the tumor sample as compared to an untreated control sample from the same subject indicates the absence of a PHF5A and/or SF3B1 mutation. In other cases, the absence of a decrease or an increase in the growth, volume, or size indicates the presence of a PHF5A and/or SF3B1 mutation.

In some embodiments, the methods provided herein further comprise administering a treatment to the subject having or suspected of having a neoplastic disorder based on the presence or absence of a mutation. Methods of treatment are described in section E (below).

In certain embodiments, determining or identifying a mutation in PHF5A may comprise sequencing a PHF5A protein, or the gene encoding PHF5A, in a sample from the patient. In some embodiments, determining or identifying a mutation in SF3B1 comprises sequencing an SF3B1 protein, or the gene encoding SF3B1, in a sample from the patient.

In some embodiments, a method of identifying a splice modulator capable of overcoming a PHF5A and/or SF3B1 mutation is provided, comprising providing a tumor sample from a subject identified as having a mutation in PHF5A (particularly a mutation at position Y36) and/or in SF3B1 (particularly a K1071E mutation, an R1074H mutation, and/or a V1078A or V1078I mutation), contacting the sample with the putative splice modulator, and measuring the growth of the tumor sample. If the tumor sample has reduced growth relative to an untreated sample, then a splice modulator capable of overcoming a PHF5A and/or SF3B1 mutation has been identified.

### E. Methods of Treatment

In various embodiments, provided herein are methods for treating a subject with a neoplastic disorder or suspected of having a neoplastic disorder. In certain embodiments, provided herein are methods for treating a subject diagnosed with a neoplastic disorder. In some embodiments, the neoplastic disorder may be a hematological malignancy, a solid tumor, or a soft tissue sarcoma. In some embodiments, the neoplastic disorder is a cancer associated with one or more mutations in the spliceosome.

In some embodiments, the neoplastic disorder is a hematological malignancy. As used herein, the term “hematological malignancy” refers to a proliferative disorder such as a cancer that affects the circulatory system, e.g., blood, bone marrow, and/or lymph nodes. Examples of hematological malignancies include, but are not limited to, myelodysplastic syndromes, chronic lymphocytic leukemia, acute lymphoblastic leukemia, chronic myelomonocytic leukemia, and acute myeloid leukemia.

In some embodiments the neoplastic disorder is a solid tumor. As used herein, the term “solid tumor” refers to a proliferative disorder such as a cancer that forms an abnormal tumor mass in a tissue that usually does not contain cysts or liquid areas, such as a sarcoma, carcinoma, and/or lymphoma. Exemplary conditions include, but are not limited to, colon cancer, pancreatic cancer, endometrial cancer, ovarian cancer, breast cancer, uveal melanoma, gastric cancer, cholangiocarcinoma, and lung cancer, or any subset thereof.

In some embodiments, the condition being treated is myelodysplastic syndrome (MDS) or another dysplasia syndrome.

In certain embodiments, the neoplastic disorder is a soft tissue sarcoma. As used herein the term “soft tissue sarcoma” refers to a type of cancer that originates in the soft tissues of a subject's body. The soft tissue may include muscle, fat, blood vessels, nerves, fibrous tissue, surrounding joints including tendons or deep skin tissue. A large variety of sarcomas can occur in these areas, and they can occur in any part of the body. Non-limiting examples may include, leiomyosarcoma, liposarcoma, fibroblastic sarcomas, rhabdomyosarcomas, and synovial sarcomas, or any variant thereof.

In various embodiments, provided herein are methods for treating a subject having or suspected of having a neoplastic plastic disorder lacking a mutation in PHF5A, as well as methods for treating a subject having or suspected of having neoplastic plastic disorder having a mutation in PHF5A and/or SF3B1.

Detailed descriptions of mutations in PHF5A and SF3B1_ and methods of detecting mutations in the proteins or the genes encoding them, are provided above.

In various embodiments, a method of treatment comprises detecting a mutation or absence of a mutation in PHF5A and/or SF3B1. In some embodiments, the method comprises administering a splicing modulator to a subject lacking a mutation in PHF5A. In some embodiments, the method comprises administering a splicing modulator to a subject lacking a mutation in PHF5A and in SF3B1.

In some embodiments, a subject diagnosed with a neoplastic disorder is treated using a splicing modulator. In other embodiments, provided herein are methods for treating a subject having or suspected of having a neoplastic plastic disorder, comprising detecting the absence of a mutation in PHF5A in the subject and administering a splicing modulator to the subject lacking a mutation in PHF5A. In other embodiments provided herein are methods for treating a subject having a neoplastic disorder, comprising obtaining a biological sample from the subject, determining that the sample from the subject does not contain a mutation in PHF5A, and administering a therapeutically effective amount of a splicing modulator to the subject. In some embodiments, the sample is determined not to have a mutation at position Y36. In some embodiments, the sample does not have a Y36A, Y36C, Y36S, Y36F, Y36W, Y36E, or Y36R mutation. In some embodiments, the subject is then administered a splicing modulator. In some embodiments, the splicing modulator is a herboxidiene, pladienolide, spliceostatin, sudemycin, or derivative or analog thereof.

In some embodiments, the sample from the subject is further assessed to determine whether it contains a mutation in SF3B1 before treatment. For example, the sample may be assessed to determine whether a mutation at one or more of positions K1071, R1074, and V1078 in SF3B1 is present. In some embodiments, a mutation is not present at one or more of positions K1071, R1074, and V1078. In some embodiments, the subject is then administered a splicing modulator. In some embodiments, the splicing modulator is a herboxidiene, pladienolide, spliceostatin, sudemycin, or derivative or analog thereof.

In some embodiments, the sample from the subject is determined to have a Y36 mutation in PHF5A and/or a mutation at one or more of positions K1071, R1074, and V1078 in SF3B1. In some embodiments, the PHF5A mutation is a Y36A, Y36C, Y36S, Y36F, Y36W, Y36E, or Y36R mutation and the mutation in SF3B1 is selected from one or more of a K1071E mutation, an R1074H mutation, and/or a V1078A or V1078I mutation. In some embodiments, a subject comprising at least this mutation pattern is not administered a splicing modulator. In some embodiments, the subject is administered an alternate cancer treatment (also referred to as an alternate anti-neoplastic agent), e.g., a cytotoxic agent, antibody, cell cycle regulatory agent, apoptotic agent, necrotic agent, or other agent that does not target the spliceosome.

In some embodiments, provided herein are methods for treating, monitoring, and/or adjusting treatment of a subject having or suspected of having a neoplastic plastic disorder. In some embodiments, the method comprises detecting the absence of a mutation in PHF5A in a first sample from the subject, administering a splicing modulator to the subject lacking a mutation in PHF5A, obtaining an additional sample from the subject after the first treatment or after several rounds of treatment, determining the presence or absence of a mutation in PHF5A in the second sample, and administering a further dose of the splicing modulator if a mutation is still absent. In some embodiments, the mutation in PHF5A is at position Y36. In some embodiments, the splicing modulator selected from herboxidiene, pladienolide, spliceostatin, sudemycin, or derivative or analog thereof.

In some embodiments, the samples are also checked for mutations in SF3B1. In some embodiments, the samples are checked for mutations at one or more of positions K1071, R1074, and V1078 in SF3B1. In some embodiments, a mutation is not present at one or more of these positions in SF3B1 (nor at position Y36 in PHF5A) and the subject is administered a splicing modulator selected from herboxidiene, pladienolide, spliceostatin, sudemycin, or derivative or analog thereof.

In further embodiments, a mutation in PHF5A is detected in the second sample after administering the splicing modulator. In some embodiments, the mutation is at position Y36. In some embodiments, the PHF5A mutation is a Y36A, Y36C, Y36S, Y36F, Y36W, Y36E, or Y36R mutation. In some embodiments, a mutation is detected in the second sample at one or more of positions K1071, R1074, and V1078 in. In these embodiments, spliceosome treatment is discontinued and the subject is not administered a further dose of the splicing modulator. In some embodiments, if a mutation in PHF5A is detected after administering the splicing modulator, the subject is administered an alternative cancer treatment that does not target the spliceosome.

In various embodiments, the process of obtaining samples and screening for mutations in PHF5A and/or SF3B1 is repeated one or more additional times throughout the treatment regimen. In some embodiments, continued treatment is contingent on the presence or absence of mutations identified in the additional samples according to the protocols described above.

In certain embodiments, provided herein are methods for identifying a subject having a neoplastic disorder responsive to a splicing modulator. In other embodiments, provided herein are methods for identifying a subject having a neoplastic disorder responsive to a splicing modulator comprising obtaining a sample from the subject, and detecting the absence of a mutation in PHF5A and/or SF3B1. In a further embodiment, the subject is identified as having a treatment-responsive neoplastic disorder when a mutation in the PHF5A and/or is not detected. In further embodiments, the subject lacking a PHF5A mutation is administered a splicing modulator. In some embodiments, the subject lacking a PHF5A and SF3B1 mutation is administered a splicing modulator. In certain embodiments, provided herein are methods for identifying a subject having a neoplastic disorder responsive to a splicing modulator comprising obtaining a sample from the subject, and detecting the absence of a mutation in PHF5A and/or SF3B1, wherein the subject is identified as having a treatment-responsive neoplastic disorder when a mutation in PHF5A and/or SF3B1 is not detected. The method may further comprise administering a splicing modulator to the subject.

In various embodiments, a subject lacking a mutation in PHF5A is administered one or more types of splicing modulators, alone or in combination with another cancer treatment not targeting the spliceosome. In some embodiments, the subject lacking a mutation in PHF5A is administered one, two, three, four, five, or more splicing modulators. Suitable therapeutically-effective dosages and dosing regimens may be selected by the skilled artisan depending on the patient and oncologic condition to be treated and other factors recognized in the art.

In some embodiments, the subject lacking a mutation is administered an SF3131 modulator. In other embodiments, the subject lacking a mutation is administered a PHF5A modulator. See section B, above, for a more detailed description of splicing modulators.

In certain embodiments, a subject lacking a mutation in PHF5A can be administered a pladienolide or a derivative, a spliceostatin or a derivative, a herboxidiene or a derivative, a thailanstatin or a derivative, or any combination thereof. In some embodiments, a subject determined to lack a mutation in PHF5A can be administered a pladienolide and/or a spliceostatin, or a herboxidiene, or a thailanstatin. In other embodiments, a subject determined to lack a mutation in PHF5A can be administered a spliceostatin and/or a pladienolide, or a herboxidiene, or a thailanstatin. In another embodiment, a subject determined to lack a mutation in PHF5A can be administered a herboxidiene and/or a spliceostatin, or a pladienolide, or a thailanstatin.

In some embodiments, a subject determined to lack a mutation in PHF5A can be administered pladienolide B, pladienolide D, E7107, or a pladienolide modulator as shown in table 1, or a combination thereof. In other embodiments, a subject determined to lack a mutation in PHF5A can be administered FR901463, FR901464, FR901465, meayamycin, meayamycin B, spliceostatin A, sudemycin C, sudemycin C1, sudemycin D1, sudemycin D6, sudemycin E, or sudemycin F, or a combination thereof. In further embodiments a subject determined to lack a mutation in PHF5A can be administered herboxidiene or a derivative.

In other embodiments, a subject lacking a mutation in PHF5A is co-administered a splicing modulator with one or more other oncology treatments.

In various embodiments, the methods provided herein comprise detecting a mutation in PHF5A. In some embodiments, the subject has been determined to have a mutation in PHF5A. In some embodiments, the subject has been determined to have a mutation in or near the PHF5A-SF3B1 interface. In some embodiments, specific PHF5A mutations include a Y36 mutation. In certain embodiments, the methods provided herein detect a Y36 mutation in PHF5A. In certain embodiments, the methods provided herein detect a Y36A, Y36C, Y36S, Y36F, Y36W, Y36E, or Y36R mutation. In specific embodiments, the methods provided herein detect a Y36C mutation.

In various embodiments when a PHF5A mutation (e.g., a Y36 mutation) and/or an SF3B1 mutation is detected, any anti-neoplastic agent that does not target the spliceosome may be used as an alternative treatment for the neoplastic disorder. In addition, such treatments may be used as adjuncts to treatment with a splice modulator in subjects who lack a PHF5A mutation.

Suitable alternative treatments may be used alone or in combination. In some embodiments, the alternative anti-neoplastic agent may be a cytotoxic agent and/or a cytostatic agent. Non-limiting examples of cytotoxic and/or cytostatic agents include Anastrozole, Azathioprine, Bcg, Bicalutamide, Chloramphenicol, Ciclosporin, Cidofovir, Coal tar containing products, Colchicine, Danazol, Diethylstilbestrol, Dinoprostone, Dithranol containing products, Dutasteride, Estradiol, Exemestane, Finasteride, Flutamide. Ganciclovir, Gonadotrophin, chorionic Goserelin, Interferon containing products (including peginterferon), Leflunomide, Letrozole, Leuprorelin acetate, Medroxyprogesterone, Megestrol, Menotropins, Mifepristone, Mycophenolate mofetil, Nafarelin, Oestrogen containing products, Oxytocin (including syntocinon and syntometrine), Podophyllyn, Progesterone containing products, Raloxifene, Ribavarin, Sirolimus, Streptozocin, Tacrolimus, Tamoxifen, Testosterone, Thalidomide, Toremifene, Trifluridine, Triptorelin, Valganciclovir, and Zidovudine.

In some embodiments, the alternative anti-neoplastic agent may be a proteasome inhibitor. In some embodiments, the proteasome inhibitor may be a pan-cytotoxic inhibitor. Non-limiting examples of protease inhibitors include bortezomib (Velcade®), carfilzomib (Kyprolis®), ixazomib (Ninlaro®), thalidomide (Thalomid®), pomalidomide (Pomalyst®), disulfiram, epigallocatechin-3-gallate, marizomib (salinosporamide A), oprozomib (ONX-0912), delanzomib (CEP-18770), epoxomicin, MG132, and beta-hydroxy beta-methylbutyrate.

In certain embodiments, the methods disclosed herein further comprise determining whether the subject has a cancer prior to treatment. In some embodiments, this determination is made by identifying one or more of the following SF3B1 mutations: E622D, E622K, E622Q, E622V, Y623C, Y623H, Y623S, R625C, R625G, R625H, R625L, R625P, R625S, N626D, N626H, N626I, N626S, N626Y, H662D, H662L, H662Q, H662R, H662Y, T663I, T663P, K666E, K666M, K666N, K666Q, K666R, K666S, K666T, K700E, V701A, V701F, V701I, I704F, I704N, I704S, I704V, G740E, G740K, G740R, G740V, K741N, K741Q, K741T, G742D, D781E, D781G, and/or D781N. In certain embodiments, the SF3B1 mutations include K700E, K666N, R625C, G742D, R625H, E622D, H662Q, K666T, K666E, K666R, G740E, Y623C, T663I, K741N, N626Y, T663P, H662R, G740V, D781E, and/or R625L. In certain embodiments, the subject identified as having cancer is then screened for resistance to splice modulating agents prior to treatment according to the methods described above. In some embodiments, a subject identified as having a cancer and having a cancer responsive to treatment with a splice modulating agent is then treated according to the methods described above.

In various embodiments, provided herein are methods for determining a treatment regime for a subject having or suspected of having a neoplastic disorder. In certain embodiments, the methods comprise identifying the presence or absence of a mutation in PHF5A. and/or SF3B1. In some embodiments, a treatment regimen comprising a splicing modulator is indicated when a mutation is absent. In other embodiments, an alternative cancer treatment is indicated when a mutation is present.

In various embodiments, provided herein are methods of monitoring mutation status in a subject during treatment of a neoplastic disorder. In some embodiments, the methods include detecting the absence of a mutation in PHF5A in the subject before or during treatment. For example, in some embodiments, the absence of a mutation in PHF5A before and/or during treatment indicates that the subject may be responsive to a splicing modulator. In other embodiments, the presence of a mutation before and/or during treatment may indicate that an alternative treatment that does not target the spliceosome is needed. In some embodiments, the methods provided herein include detecting the absences of a mutation in PHF5A before treatment, administering a splicing modulator to the subject, and monitoring the mutation status during treatment. In some embodiments, the method further comprises detecting the absence of a mutation in PHF5A during treatment with a splicing modulator and deciding to continue with treatment. The absence of a mutation in PHF5A indicates that the subject may be administered a further dose of a splicing modulator. In some embodiments, the method further comprises detecting the presence of a mutation in PHF5A during treatment with a splicing modulator and deciding to discontinue treatment and/or switch to an alternate cancer treatment. For example, the presence of a mutation in PHF5A may indicate that treatment with the splicing modulator should be terminated and an alternative treatment, as described herein, should be administered.

In some embodiments, the methods provided herein comprise monitoring for the presence or absence of a mutation in PHF5A throughout treatment. In some embodiments, the methods provided herein further comprise also monitoring for the presence or absence of a mutation in SF3B1 throughout treatment. In some embodiment, the methods provided herein comprise checking for the presence or absence of a mutation in PHF5A and/or SF3B1 after each treatment cycle with a splicing modulator.

In various embodiments, the disclosure herein provides splice modulators for use in the treatment of neoplastic disorders, wherein the splice modulators are indicated for use when mutations in PHF5A and/or SF3B1 are present or absent as indicated previously. In various embodiments, the disclosure herein provides splice modulators for use in the manufacture of medicaments for treating neoplastic disorders, wherein the splice modulators are indicated for use when mutations in PHF5A and/or SF3B1 are present or absent as indicated previously. In various embodiments, the disclosure herein provides mutations in PHF5A and/or SF3B1 for use in treating neoplastic disorders, where splice modulators are indicated for treatment depending on the presence or absence of the mutations in PHF5A and/or SF3B1.

### F. Kits

Also disclosed herein, in various embodiments, is a kit comprising a reagent that detects a mutation in PHF5A and/or SF3B1. One skilled in the art will recognize components of kits suitable for carrying out a method (or methods) of the present disclosure. For example, a kit may include one or more containers, each of which is suited for containing one or more reagents or other means for detecting mutations in PHF5A and/or SF3B1, instructions for detecting mutations in PHF5A and/or SF3B1 using the kit, and optionally instructions for carrying out one or more of the methods described herein after identifying the presence or absence of such mutations.

In some instances, the kit may also include one or more vials, tubes, bottles, dispensers, and the like, which are capable of holding one or more reagents needed to practice the present disclosure.

Instructions for kits of the present disclosure may be affixed to packaging material, included as a package insert, and/or identified by a link to a website. While the instructions are typically written or printed materials, they are not limited to such, Any medium capable of storing such instructions and communicating them to an end user is contemplated by the present disclosure. Such media include, but are not limited to, electronic storage media (e.g., magnetic discs, tapes, cartridges, chips), optical media (e.g., CD ROM), and the like. As used herein, the term “instructions” can include the address of an Internet site that provides the instructions. An example of this can include a kit that provides a web address where the instructions can be viewed and/or from which the instructions can be downloaded. In other instances, kits of the present disclosure may comprise one or more computer programs that may be used in practicing the methods of the present disclosure. For example, a computer program nay be provided that takes the output from microplate reader or realtime-PCR gels or readouts and prepares a calibration curve from the optical density observed in the wells, capillaries or gels, and compares these densitometric or other quantitative readings to the optical density or other quantitative readings in wells, capillaries, or gels with test samples.

In some embodiments, the kit can comprise instructions for use to detect a mutation. In other embodiments, the kit can comprise a reagent that detects a mutation in PHF5A, and instructions for use to detect a mutation. The kit may further comprise a reagent for detecting a mutation in SF3B1. In some embodiments, the kit is used to detect a Y36C mutation in PHF5A and/or a K1071, R1074, or a V1078 mutation in SF3B1, or a combination thereof. In specific embodiments, the kit described herein is used to detect the presence or absence of a Y36C mutation in PHF5A and/or a K1071 mutation in SF3B1. In another specific embodiment, the kit described herein is used to detect the presence or absence of a Y36C mutation in PHF5A and/or a R1074 mutation in SF3B1. In yet another specific embodiment, the kit described herein is used to detect the presence or absence of a Y36C mutation in PHF5A and/or a V1078 mutation in SF3B1. In other embodiments, the kit is used to detect the presence or absence of a Y36C mutation in PHF5A. In some embodiments, the kit is used to detect the presence or absence of a K1071 mutation in SF3B1. In other embodiments, the kit is used to detect the presence or absence of a R1074 mutation in SF3B1. In other embodiments, the kit is used to detect the presence or absence of a V1078 mutation in SF3B1.

### EQUIVALENTS

It will be readily apparent to those skilled in the art that other suitable modifications and adaptations of the methods of the invention described herein are obvious and may be made using suitable equivalents without departing from the scope of the disclosure or the embodiments. Having now described certain compounds and methods in detail, the same will be more clearly understood by reference to the following examples, which are included for purposes of illustration only and are not intended to be limiting.

### Examples

The following examples serve to illustrate, and in no way limit, the present disclosure.

### 1. Methods

### 1.1. Materials

Parental HCT116 cells were obtained from ATCC and cultured in RPMI 1640 medium (Thermo Fisher, GIBCO #11875) supplemented with 10% FBS. Parental Panc0504 cells were obtained from ATCC and cultured in GIBCO RPMI 1640 medium (Thermo Fisher, GIBCO #11875) supplemented with glucose (to 4.5 g/L final), HEPES (10 mM final), sodium pyruvate (1 mM final), human insulin (10 μg/ml final) and 15% FBS. Cell line authentication was achieved by genetic profiling using polymorphic short tandem repeat (STR) loci (ATCC). All cell lines were free of mycoplasma contamination. Lenti-X-293T cells (Clontech Laboratories, Inc. Cat #632180), a cell line for lentiviral packaging, was maintained in Dulbecco's modified Eagle's medium (Thermo Fisher, GIBCO #11965) containing 10% fetal bovine serum and 4 mM L-glutamine. WT PHF5A cDNA was obtained from Genecopoeia and cloned into a pDONR 221 vector (Thermo Fisher). Sequence verified positive clones were cloned into pLenti6.3N5 vector (Thermo Fisher) through LR recombination. Mutagenesis of Y36 and V37 were carried using Agilent Quickchange II kit following manufacturer's recommendation using the PHF5A WT plasmid. All primers used for mutagenesis were designed used the QuikChange Primer Design tool by Agilent. Verified positive clones of PHF5A Y36 or V37 variants were used for lentivirus production using X293T cells. Parental HCT116 cells and Panc0504 cells were then infected with virus containing medium and selected with Blasticidin S (Thermo Fisher) at 10 μg/ml for one week. Engineered cell lines were maintained in the same medium without antibiotics. The following primary antibodies were used at 1:1000 dilution for western blot analysis in LI-COR buffer (LI-COR): α-SF3B1 mouse monoclonal antibody (MBL, D221-3), α-SF3B3 rabbit polyclonal antibody (Protein Tech, 14577-1-AP), α-SF3B4 goat polyclonal antibody (Santa Cruz, 14276), α-SF3B6/p14 rabbit polyclonal antibody (Protein Tech, 12379-1-AP), α-PHF5A rabbit polyclonal antibody (Protein Tech, 15554-1-AP). α-GAPDH rabbit polyclonal antibody (Sigma, G9545) was used at 1:10,000. Anti-rabbit and anti-goat IRDye-800CW secondary antibody (LI-COR) was used at 1:5000 dilution and anti-mouse IRDye-680LT secondary antibody (LI-COR) was used at 1:20,000 dilution. Western blot was imaged using Odyssey V3.0 imager (LI-COR).

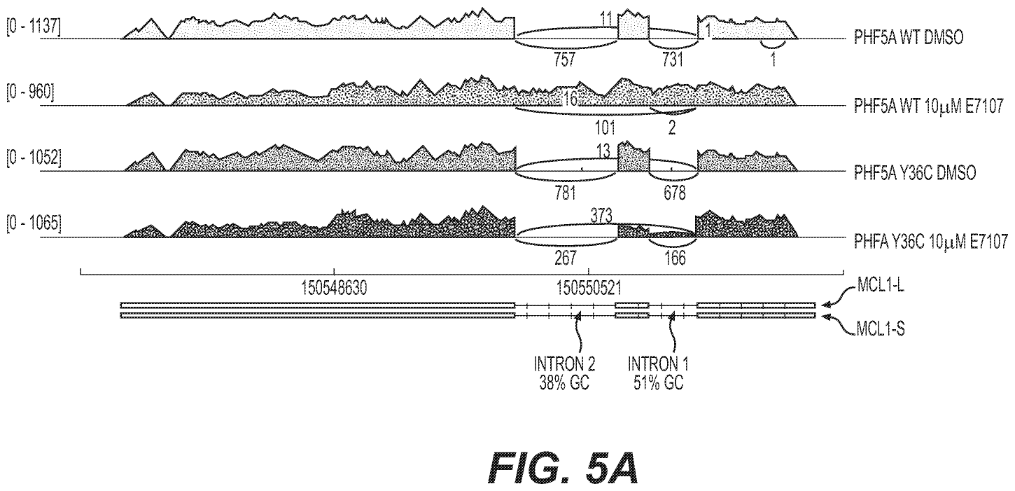

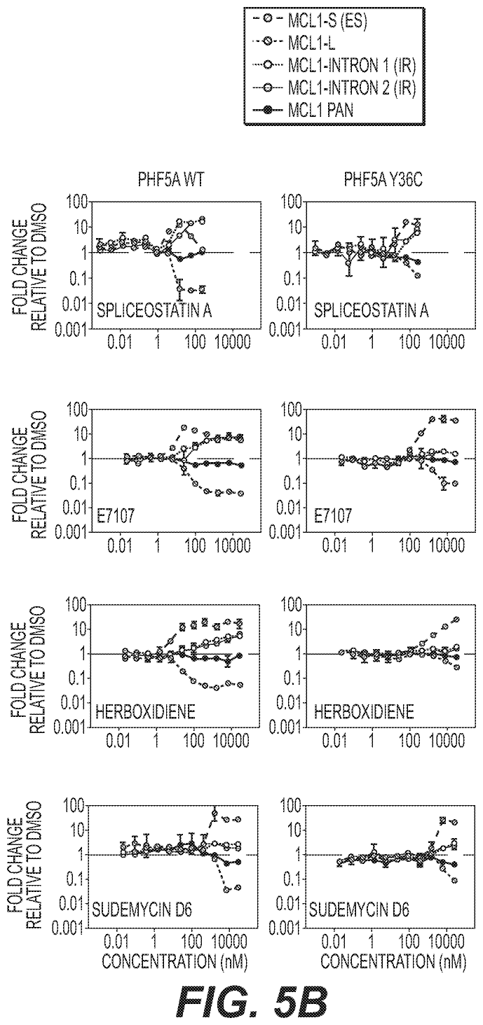

FIG. 5 shows that PHF5A-Y36C alters splicing modulators effects toward MCL1 splicing. FIG. 5A depicts a representative sashimi plot of the production of different MCL1 isoforms under indicated treatment from either WT or Y36C PHF5A over-expressing cells. FIG. 5B shows a taqman gene expression analysis of indicated MCL1 isoforms in either WT (left panel) or Y36C (right panel). PHF5A over-expressing cells treated with splicing modulators. Error bar indicates standard deviation, n=4.

### 1.2. Compounds

Bortezomib (PS-341) was purchased from LC Laboratories (Cat. No. B-1408, Lot: BBZ-112). E7107 and ³H labelled Pladienolide probe were provided by Eisai Co. Ltd. and their synthesis was previously reported (Kotake et al., The FEBS journal 278, 4870-80 (2011)). Herboxidiene was also provided by Eisai Co. Ltd. Spliceostatin A and Sudemycin D6 were synthesized in house following established procedures (Ghosh and Chen, Organic letters 15, 5088-91 (2013); Lagisetti et al., Journal of medicinal chemistry 56, 10033-44 (2013)). For splicing modulators, the compound identity and purity was assessed by LC/MS and proton NMR. Purity was determined using a Waters H class Acquity ultra performance liquid chromatography system with an XSelect CSH C18, 1.7 μm 2.1×50 mm column, a flow rate of 0.8 mL/min at 20° C. Injections consisted of 1 μL of 1 mM sample in DMSO over a gradient from 5% acetonitrile and 0.1% formic acid to 90% acetonitrile and 0.1% formic acid over a time span of 2.5 mins. Purity for each compound was determined from the integrated UV absorbance peak. Masses were detected in the positive ion scan and correspond to those predicted by their formula weight. The detector conditions were: capillary voltage 3.25 kV, cone voltage 30 V, source temperature 150° C., desolvation temperature 500° C., desolvation gas 1000 L/hr, cone gas 100 L/hr. Single ion recording was used to determine quantification of samples. The data were acquired over scan range from m/z=100-1000 in 0.2 s and processed using QuanLynx software. Proton NMR spectra were acquired for each compound on a Bruker Ascend 400 MHz spectrometer to further assess the identity and purity of the samples. The indicated solvents correspond to those used in previous publications (pyridine for E7107 (Kotake et al., Nature chemical biology 3, 570-5 (2007)), chloroform for spliceostatin A (Ghosh and Chen, Organic letters 15, 5088-91 (2013)) and sudemycin D6 (Lagisetti et al., Journal of medicinal chemistry 56, 10033-44 (2013)), and methanol for herboxidiene (Ghosh and Li, Organic letters 15, 5088-91 (2013)). The acquired spectra match previous data reported for these compounds.

### 1.3. Resistant Clone Generation, Whole Exome Sequencing Sample Preparation, Data Process and Identification of Candidate Mutations

2.5 million HCT116 cells were seeded in each 10 cm dish and treated with indicated dosages of splicing modulators for 2 weeks. Compounds were refreshed every 4 days. When needed, confluent dishes were split 1:3 and cells were allowed to recover overnight without splicing modulator treatment after re-seeding. At the end of the compound selection period, surviving individual clones were picked and transferred to 12-well plates. Individual resistant clones were further expanded without splicing modulator treatment and 1 million cells from each clone were pelleted for genomic DNA extraction using the DNeasy Blood & Tissue Kit from Qiagen. Whole exome sequencing (WXS) libraries were generated by Novogene Corporation using Agilent SureSelect Human All Exon V6 kit and sequenced on Illumina HiSeq platform. 12G raw data were gathered for each sample. WXS reads were then aligned to hg19 by BWA-MEM (Shi et al., Nature biotechnology 33, 661-7 (2015)) and somatic mutations were identified with MuTect2 (Schenone et al., Nature chemical biology 9, 232-40 (2013)) through Sentieon pipeline (Wacker et al., Nature chemical biology 8, 235-7 (2012)) by pairing resistant clone with parental cell lines. As the resistant clones for WXS were selected, the allele frequencies for the mutations which are responsible for the resistance should be high. Non-silent mutations (among the H3 curated spliceosome genes) with allele frequency higher than 0.2 were focused on.

### 1.4. Cell Titer-Glo Luminescent Cell Viability Assay for Growth Inhibition Analysis

For CellTiter-Glo analysis, 500 cells were seeded in each well of a 384-well plate the day before compound addition. An 11 part serial dilution was used starting with a top final dosage of 10 μM for ten additional doses. DMSO percentage was maintained throughout and a DMSO only control was included. 72 hours post compound addition; CellTiter-Glo reagent was added to the medium, incubated and assayed on EnVision Multilabel Reader (PerkinElmer). The luminescence value from each treatment sample was normalized to the average value of the respective DMSO control. The dosage response curve plots were generated using Graphpad Prism 6 and fit using nonlinear regression analysis and the log (inhibitor) vs. response—Variable slope (four parameters). For heatmap summarization of IC50 shifts, IC50 value were extracted from dosage response curves and the fold changes of IC50 values in PHF5A variants expressing lines over that of the WT lines were calculated and plotted using TIBCO Spotfire software. For IC50s greater than the top dosage, the values were arbitrarily set at 10 μM. Unsupervised clustering analysis was performed in TIBCO Spotfire using the following default parameters: Clustering method: UPGMA; Distance measure: Euclidean; Ordering weight: Average value; Normalization: (None); Empty value replacement: Constant value: 0.

### 1.5. Cell Proliferation Assay

1000 cells of indicated genotypes were seeded in 96-well clear bottom plates (Corning, #3904) and HD phase-contrast image was captured every 4 hours with 4× objective lens using IncuCyte ZOOM System (Essen BioScience). Collected images were analyzed with IncuCyte ZOOM Software (2016A) (Essen BioScience) to calculate the confluency percentage. Analyzed data were graphed with Graphpad Prism 6, n=5.

### 1.6. Immunofluorescence

One million cells of indicated genotypes were seeded onto Corning BioCoat Fibronectin 22 mm cover-slips (Fisher Scientific 08-774-386) in 6 well plates. After 2 days, cells were fixed with 4% paraformaldehyde/PBS for 20 mins at room temperature (RT). After 3×PBS wash, cells were permeabilized with 0.1% Triton X-100/PBS for 20 mins at RT. After 3×PBS wash, cells were blocked with 5% FBS/PBS for 1 hour at RT and incubated with α-SF3B1 mouse monoclonal antibody (MBL, D221-3) or α-SC35 mouse monoclonal antibody (Abcam, ab11826) at 1:50 dilution in 5% FBS/PBS in cold room overnight. On the second day, coverslips were washed with PBS three times and incubated with Alexa Fluor 488 anti-mouse secondary antibody (Thermo Fisher Cat #: A-11029) at 1:500 dilution in 5% FBS/PBS at RT in dark for 1 hour. Coverslips were then washed with PBS three times and mounted using ProLong Gold Antifade Mountant with DAPI (Thermo Fisher, P36935). Slides were imaged with 10× objective on Olympus IX-81 inverted fluorescence microscope and imaged captured and processed with Metamorph for Olympus.

### 1.7. Cell Lysis and Nuclear Extract Preparation

For western blot analysis, cell pellets were extracted using RIPA buffer supplemented with proteasome complete protease inhibitor cocktail and PhosStop phosphatase inhibitor cocktail (Roche Life Science). Lysates were then centrifuged for 10 min at top speed; the supernatants were subjected to SDS-PAGE. For nuclear extract preparation, cells were first washed and then scraped into PBS. After centrifugation, cell pellets were resuspended in 5 packed cell volume (PCV) of hypotonic buffer (10 mM HEPES, pH7.9, 1.5 mM MgCl2, 10 mM KCl, 0.2 mM PMSF, 0.5 mM DTT) and centrifuged at 3000 rpm for 5 min. Cell pellets were resuspended in 3 PCV of hypotonic buffer and swelled on ice for 10 min. Swollen cells were then lysed using a dounce homogenizer and spun at 4000 rpm for 15 min at 4° C. The pellets contained the nuclei and were suspended with half packed nuclei volume (PNV) of low salt buffer (20 mM HEPES, pH7.9, 1.5 mM MgCl2, 20 mM KCl, 0.2 mM EDTA, 25% glycerol, 0.2 mM PMSF, 0.5 mM DTT) gently. Half PNV of high salt buffer (20 mM HEPES, pH7.9, 1.5 mM MgCl2, 1.4 M KCl, 0.2 mM EDTA, 25% glycerol, 0.2 mM PMSF, 0.5 mM DTT) was then added and mixed gently. The lysates were rocked for 30 min in cold room before centrifuged at 10,000 rpm for 30 min at 4° C. The supernatants contained the nuclear extracts and were dialyzed for 4 hours using Slide-A-Lyzer dialysis cassettes with 30,000 MWCO cutoff in dialysis buffer (20 mM HEPES, pH7.9, 0.2 mM EDTA. 20% glycerol, 0.2 mM PMSF, 0.5 mM DTT) with a change of buffer after 2 hours. The nuclear extract was then aliquoted and flash frozen.

### 1.8. In Vitro Splicing Assay

The following Ad2-derived (Pellizzoni et al., Cell 95, 615-24 (1998)) and subsequently modified (Corrionero et al., Genes & development 25, 445-59 (2011)) sequence actctcttccgcatcgctgtctgcgagggccagctgttggggtgagtactccctctcaaaagcgggcatgacttctgcgctaaga ttgtcagtttccaaaaacgaggaggatttgatattcacctggcccgcggtgatgcctttgagggtggccgcgtccatctggtcag aaaagacaatctttttttgttgtcaagattgcacgtctagggcgcagtagtccagggtttccttgatgatgtcatactaatcctgtcc cttttttttccacagctcgcggttgaggacaaactcttcgcggtctttccagtactcttggatcggaaacccgtcggcctccgaacg (SEQ ID NO: 3) (intron in italic and underlined) was cloned into the pGEM-3Z vector (Promega) using 5′ EcoRI and 3′ XbaI restriction sites. The FtzΔi plasmid (Luo & Reed, Proceedings of the National Academy of Sciences of the United States of America 96, 14937-42 (1999)) was obtained from Robin Reed. The pGEM-3Z-Ad2.1 and FtzΔi plasmids were linearized using XbaI and EcoRI, respectively, purified, resuspended in TE buffer, and used as a DNA template in the in vitro transcription reaction. The Ad2.1 pre-mRNA and Ftz mRNA were generated and purified using MEGAScript T7 and MegaClear kits, respectively (Invitrogen). 20 μL splicing reactions were prepared using 80 μg nuclear extracts, 20U RNAsin Ribonuclease inhibitor (Promega), 20 ng Ad2.1 pre-mRNA and 2 ng Ftz mRNA (internal control). After a 15 min pre-incubation with indicated compound, activation buffer (0.5 mM ATP, 20 mM creatine phosphate, 1.6 mM MgCl2) was added to initiate splicing, and the reactions were incubated for 90 min at 30° C. RNA was extracted using a modified protocol from a RNeasy 96 Kit (Qiagen). The splicing reactions were quenched in 350 μL Buffer RLT Plus (Qiagen), and 1.5 volume ethanol was added. The mixture was transferred to an RNeasy 96 plate, and the samples were processed as described in the kit protocol. RNA was diluted 1/100 with dH₂O. 10 μL RT-qPCR reactions were prepared using TaqMan RNA-to-C_{T} 1-step kit (Life Technologies), 2 μL diluted splicing reactions, 0.5 μL Ad2 (forward: ACTCTCTTCCGCATCGCTGT (SEQ ID NO: 4); reverse: CCGACGGGTTTCCGATCCAA (SEQ ID NO: 5); probe: CTGTTGGGCTCGCGGTTG (SEQ ID NO: 6)), and 0.5 μL Ftz (forward: TGGCATCAGATTGCAAAGAC (SEQ ID NO: 7); reverse: ACGCCGGGTGATGTATCTAT (SEQ ID NO: 8); probe: CGAAACGC ACCCGTCAGACG (SEQ ID NO: 9)) mRNA primer/probe sets. The Ad2 Ftz probes are from IDT and labeled with FAM acceptor with ZEN quencher and the Ftz probe is labeled with Hex and ZEN quencher.

### 1.9. Scintillation Proximity Assay

Batch immobilization of anti-FLAG antibody (Sigma) to anti-mouse PVT SPA scintillation beads (PerkinElmer) was prepared as follows. For every 1.5 mg of beads, 10 μg antibody was prepared in 150 μL PBS. The antibody-bead mixture was incubated for 30 minutes at room temperature and centrifuged at 15,000 RPM for 5 minutes. 150 μL PBS was used to resuspend every 1.5 mg antibody-bead mixture. The aforementioned mini-SF3b complexes were tested for ³H-labelled pladienolide probe (Kotake et al., Nature chemical biology, 570-5 (2007)) binding. 100 μL binding reactions were prepared with 50 μL bead slurry and 0 or 50 nM protein in buffer (20 mM HEPES pH 8, 200 mM KCl, 5% glycerol). The mixture was incubated for 30 minutes, and varying concentrations of ³H-labelled pladienolide probe were added. The mixture was incubated for 30 minutes, and luminescence signals were read using a MicroBeta2 Plate Counter (PerkinElmer). Compound competition studies were performed with the WT mini-SF3b complex. 100 μL binding reactions were prepared with 50 μL bead slurry, 25 nM protein in buffer, and compounds at varying concentrations. After a 30-minute pre-incubation, 1 nM ³H-labelled pladienolide probe was added. The reactions were incubated for 30 minutes, and luminescence signals were read.

Previous prepared nuclear extracts were stored as 2.5 mg aliquots. Each aliquot was sufficient for three SPA samples and was diluted into a total volume of 1 mL PBS with phosphatase and protease inhibitors. Sufficient amount of aliquots were centrifuged at 15,000 RPM for 10 mins at 4° C. The supernatant was removed into a clean tube and kept on ice. Recombined protein complexes containing WT or Y36C PHF5A were prepared as described above. Batch immobilization of anti-SF3B1 (MBL) antibody to anti-mouse PVT SPA scintillation beads (PerkinElmer) was prepared as follows. For every 2.5 mg of nuclear extracts, 5 μg anti-SF3B1 antibody and 1.5 mg of beads were mixed in 150 μL PBS. The antibody-bead mixture was incubated for 30 mins at room temperature and centrifuged at 15,000 RPM for 5 mins. The beads were suspended and added to the prepared nuclear extracts. The slurry was incubated for 2 hours at 4° C. with gentle mixing. The beads were collected by centrifuging at 15,000 RPM for 5 mins, and washed twice with PBS+0.1% Triton X-100. After a final centrifugation step, every 1.5 mg of beads was suspended with 150 μL of PBS. 100 μL binding reactions were prepared as follows: 50 μL bead slurry, 25 μL cold competitive compound at 10 μM, and after 30 mins pre-incubation, 10 nM 3H-labelled pladienolide probe was added. The mixture was incubated for 30 mins, and luminescence signals were read using a MicroBeta2 Plate Counter (PerkinElmer).

### 1.10. Mass Spectrometry Analysis

The enriched samples were reduced with 5 mM DTT at 56° C. for 45 mins and alkylated with 20 mM Iodoacetamide at room temperature for 30 mins. The samples were run on a 4-15% Tris glycine gel and the gel was excised, de-stained and trypsin digested overnight at 30° C. Peptides were extracted with 50 μl of buffers A, B and C sequentially (Buffer A—1% formic acid and 50% acetonitrile, B—100 mM Ammonium Bicarbonate, C—100% acetonitrile). Samples were dried down using a lyophilizer and resuspended in 30 μl of running buffer A (0.1% formic acid in water). Samples were analyzed by nanocapillary liquid chromatography tandem mass spectrometry on an easy-nLC 1000 HPLC system coupled to a QExactive mass spectrometer (Thermo Scientific) using a C18 easy spray column Particle Size: 3 μm; 150×0.075 mm I.D. and the data were analyzed using Proteome discoverer 1.4.

### 1.11. Cloning, Protein Purification, and Crystallization of PHF5A

Full-length human PHF5A, containing a C40S mutation for enhanced protein stability, was synthesized and subcloned between the NdeI and EcoRI sites of pET-28a with an N-terminal His-MBP-TEV cleavable tag. Protein was expressed in BL21 (DE3) star cells grown in LB media. Cells were induced at OD₆₀₀=1.0 overnight at 16° C. with 0.5 M IPTG supplemented with 100 μM ZnCl₂. Lysate was prepared in HEPES pH 7.5, 500 mM NaCl, 1 mM TCEP, loaded onto a NTA-column, and eluted over a gradient up to 500 mM imidazole. The peak fraction was pooled and the MBP tag was cleaved by TEV protease overnight at 4° C. Cleaved MBP and excess TEV were removed by reverse NTA-column. The flow through fractions containing PHSA were concentrated and loaded onto a 16/60 Sephacryl-100 column equilibrated in 100 mM NaCl, 25 mM HEPES pH7.5, 1 mM TCEP. The peak fraction was further purified by ion exchange on a HiTrap SP HP column equilibrated in gel filtration buffer and eluted in a gradient up to 1 M NaCl. PHF5A eluted in approximately 300 mM NaCl and was concentrated to 10 mg/ml and flash frozen in liquid N2 for storage at −80° C. The resulting protein failed to crystallize but a proteolytically stable domain was obtained by limited digestion with chymotrypsin (1:1000 molar ratio) for two hours at room temperature. Cubic shaped crystals grew to final dimensions of 50×50×50 microns after a week from 2 μL+2 μL hanging drops equilibrated over a reservoir containing 100 mM CHES pH9.5, 800 mM sodium citrate and 0.5% octyl-β-glucoside. Crystals were frozen in reservoir solution supplemented with 20% ethylene glycol.

### 1.12. Structure Determination

Single wavelength anomalous diffraction (SAD) data at the zinc edge was collected by Shamrock Structures LLC at the APS beamline 21D. Crystals diffracted to 2.0 Å and the data were processed with iMosflm and xia2 in a cubic space group P2₁3 (a=b=c=82.2 Å and α=β=γ=90°) (Winter et al., Acta crystallographica. Section D, Biological crystallography 69, 1260-73 (2013); Battye et al., cta crystallographica. Section D, Biological crystallography 67, 271-81 (2011)) indicating a solvent content of 47%, assuming two molecules in the asymmetric unit. Anomalous signal extended to approximately 2.0 Å and was used to located six high-occupancy zinc anomalous sites using SHELX C/D/E (Skubak & Pannu et al., Nature communications 4, 2777 (2013); Sheldrick, Acta crystallographica. Section D, Biological crystallography 66, 479-85 (2010)), confirming two molecules in the asymmetric unit. The FOM from this initial substructure solution was 0.404 and after density modification and hand determination, the FOM improved to 0.76. Buccaneer and REFMAC5 (Murshudov et al., Acta crystallographica. Section D, Biological crystallography 53, 240-55 (1997)) auto-traced 76 residues for each monomer and an additional 13 residues were built using Coot. This model was used to refine against the native data set to 1.8 Å and after several iterative rounds of rebuilding and refinement, the final model was obtained consisting of residues 2-91 in molecule A and 3-92 in molecule B and final statistics R=0.17, R_{free}=0.20 and FOM=0.86 (Murshudov et al., Acta crystallographica. Section D, Biological crystallography 53, 240-55 (1997); Emsley et al., Acta crytallographica. Section D, Biological crystallography 66, 486-501 (2010)). The refined coordinates were deposited in the protein data bank (PDB: 5SYB).

### 1.13. Cloning and Purification of the Recombinant Protein Complex

In order to reassemble the modulator-binding site, four proteins from SF3b complex were selected based on the yeast cryo-EM structure. Truncated SF3B1, full-length SF3B3, PHF5A, and SF3B5 were synthesized and subcloned between the EcoRI and NcoI site of pFastBac1 vector. Only the HEAT repeat domain from residue 454-1304 of SF3B1 was cloned with an addition of N-terminal FLAG tag. SF3B3 and SF3B5 were with an N-terminal His-tag. Four viruses were generated and used to co-infect SF21 cells at ratio of ˜10:1. The cells were harvested after 72 hours and lysed in 40 mM HEPES pH8.0, 500 mM NaCl, 10% glycerol and 1 mM TCEP. The complex was purified by batch method, using nickel beads and FLAG beads. The eluent was concentrated and ran on a gel filtration column (superdex 200) in buffer 20 mM HEPES pH8.0, 300 mM NaCl, 10% glycerol and 1 mM TCEP. The fraction was collected, concentrated to 4 mg/mL and flash frozen in liquid N2 for storage at −80. The production of recombinant complex containing PHF5A-Y36C mutation is the same as the WT recombinant complex.

### 1.14. RNA-Seq Sample Preparation, Data Process and Identification of Differential Splice Junctions and Gene Level Venn Diagram Generation and Gene Set Enrichment

Either PHF5A WT or Y36C mutant overexpressing cells were treated with either DMSO or E7107 (100 nM and 10 μM) for 6 hours in quintuplicate before lysed in TRIzol reagent (Thermo Fisher). After phase separation, top aqueous phase was further processed using MagMAX™-96 Total RNA Isolation Kit (Thermo Fisher, AM1830) for RNA extraction. RNA quality was assessed using Agilent tapestation with RNA screen tape. RNA-seq libraries were prepared by Beijing Genomic Institute (BGI) and sequenced on Illumina Hiseq 4000 for 6G clean reads per sample. RNA-seq reads were aligned to hg19 by STAR (Dobin et al., Bioinformatics 29, 15-21 (2013)) and raw junction counts generated by STAR were used for calculating percent spliced in (PSI) to quantify splice junction usage relative to all other splice junctions that share the same splice site as described before (Darman et al., Cell reports 13, 1033-45 (2015)). Differential PSI were assessed between a pair of sample groups using moderated t-test defined in limma package (Smyth, Statistical applications in genetics and molecular biology 3, Article3 (2004)) in Bioconductor. The statistical p-values were corrected using the Benjamini-Hochberg procedure and q-values less than or equal to 0.05 were considered statistically significant. Gene IDs associated with significant splicing changes upon E7107 treatment as compared to DMSO in either PHF5A WT or Y36C cells were used for generation of the Venn Diagram using online tool (http://bioinformatics.psb.ugent.be/webtools/Venn/). PHF5A WT or Y36C specific genes identified from the Venn Diagram analysis were then subject to Gene Set Enrichment Analysis (GSEA) (http://software.broadinstitute.org/gsea/msigdb/annotate.jsp) using the Kyoto Encyclopedia of Genes and Genomes (KEGG) pathway database.

### 1.15. Exon-Skipping Versus Intron-Retention PSI Comparison for 3383 Junctions

The number of reads which cover the splice junction which excludes a given cassette exon (exon skipping reads) were compared with both the number of spliced reads which share its 3′ splice site yet have an alternative 5′ splice site bordering the cassette exon (exon inclusion reads) and the number of reads which cross the exon-intron boundary at that same 3′ splice site (intron retention reads.) These counts were summed and their fractions from the percent spliced in (PSI) for the exon skipping event, the exon inclusion event, and the intron retention event, respectively, at that locus. The PSI for all significant exon skipping events derived from the comparison between 100 nM E7107 treatment in PHF5A Y36C cells and the respective DMSO controls (3883 events) and the PSI for the intron retention junction at the same locus were plotted. For all other treatments, the PSI of the exon skipping junction and the intron retention junction for each locus were plotted in the same order. PSI is averaged over samples in quintuplicate.

### 1.16. GC Content Calculation of Significantly-Retained Intron Junctions

The set of all significant, treatment-induced exon skipping junctions were reduced to those introns (those bordering the cassette exon on their 3′ and 5′ ends, respectively) had a sequence length of at least 100, were significantly enriched in the untreated samples as “exon inclusion” events with q<0.05, and for which the intervening sequence space formed by the borders of their 3′ and 5′ ends was known be an exon in the RefSeq transcriptome annotation of length at least 50, to avoid ambiguity caused by events which skip multiple exons. The sequences of each intron and exon were divided into 100 and 50 bins of equal length strings, respectively, then the GC content (fraction of bases either ‘G’ or ‘C’) were assessed for each string. Once all intron/exon pairs have their sequence content binned in this way, the resulting mean and 95% confidence interval for each bin was assessed using 100 bootstraps of the data (up to the number of intron/exon pairs, with replacement) and drawn using a solid line and a transparent interval, respectively. The background was drawn from 10,000 random intron/exon pairs from RefSeq which satisfied the same length and boundary requirements.

### 1.17. Taqman Gene Expression Assay

8000 cells of indicated genotypes were seeded in each well of 96-well plate and allowed to settle overnight. On the second day, 11 points serial dilution (1:4 fold dilution across) of indicated compound with a top dosage of 10 μM final was added to the culturing medium. 4 hour post compound addition, culturing medium was decanted and washed once with PBS. PBS was then decanted completely from the plate and Lysis buffer (plus DNase I) from TaqMan Gene Expression Cells-to-CT Kit (Thermo Fisher, cat # AM1729) was added according to the manual. After 5 mins incubation at room temperature on the shaker, stop solution was added to each well and incubated for 2 min. Reverse transcription was set up immediately using the Cells-to-CT Kit and cDNAs were used for quantitative real time PCR analysis using Viia7 (Thermo Fisher). Each reaction is multiplexed with a FAM labelled probe targeting specific target gene splicing isoforms and a VIC labelled probe targeted 18S rRNA as loading control. Therefore, the FAM Ct value in each well was first normalized to the VIC Ct value in the same well before further normalization to the FAM/VIC ratio of DMSO treated control samples to calculate fold change over DMSO. Graphs were generated using Graphpad Prism 6, n=2. Taqman gene expression probes used in these assays are:

TABLE 2
Gene probes
MCL1-L probe set

| | |
|---|---|
| Forward Primer | ATATGCCAAACCAGCTCCTAC (SEQ ID NO: 10) |
| Probe | AGAACTCCACAAACCCATCCCAGC (SEQ ID NO: 11) |
| Reverse Primer | AAGGACAAAACGGGACTGG (SEQ ID NO: 12) |

MCL1-S probe set

| | |
|---|---|
| Forward Primer | AAAGCCAATGGGCAGGT (SEQ ID NO: 13) |
| Probe | TCCACAAACCCATCTTGGAAGGCC (SEQ ID NO: 14) |
| Reverse Primer | CCACCTTCTAGGTCCTCTACAT (SEQ ID NO: 15) |

MCL1 intron1 probe set

| | |
|---|---|
| Forward Primer | GACAAAGGAGGCCGTGAGGA (SEQ ID NO: 16) |
| Probe | GTTTGTTACGCCGTCGCTGAAA (SEQ ID NO: 17) |
| Reverse Primer | TCAGGCATGCTTCGGAAACTGGA (SEQ ID NO: 18) |

MCL1 intron2 probe set

| | |
|---|---|
| Forward Primer | GCCCCGGGGTGAATAATAATTGGTTTACT (SEQ ID NO: 19) |
| Probe | TTTCTAGGATGGGTTTGTGGAGTT (SEQ ID NO: 20) |
| Reverse Primer | CCTGATGCCACCTTCTAGGTCCTCTAC (SEQ ID NO: 21) |

pan MCL1 probe set

| | |
|---|---|
| Forward Primer | GCCAAGGACACAAAGCCAAT (SEQ ID NO: 22) |
| Probe | CTGGAGACCTTACGACGGGTTGGG (SEQ ID NO: 23) |
| Reverse Primer | AAGGCCGTCTCGTGGTT (SEQ ID NO: 24) |
| SLC25A19 mature form | Life Technologies Assay ID = Hs00222265_ml |
| probe set | |
| EIF4A1 pre-mRNA form | Life Technologies Assay ID = AJRR9DL |
| probe set | |

### 1.18. Statistics

Appropriate statistical methods and determination of statistical significance were performed as described in the above sections.

### 2. Results

### 2.1. Chemogenomic Analysis Identifies New Resistant Mutations in PHF5A and SF3B1 Against Splicing Modulators

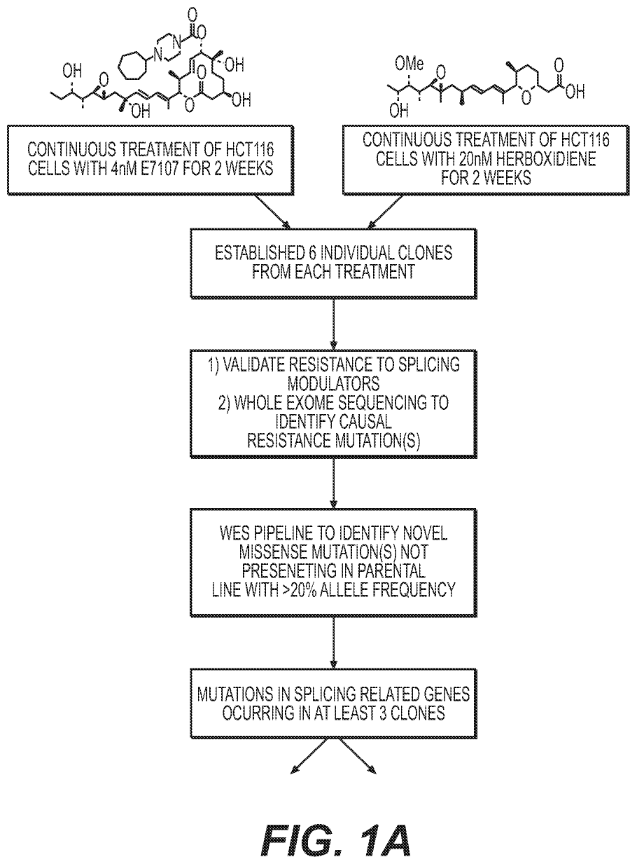

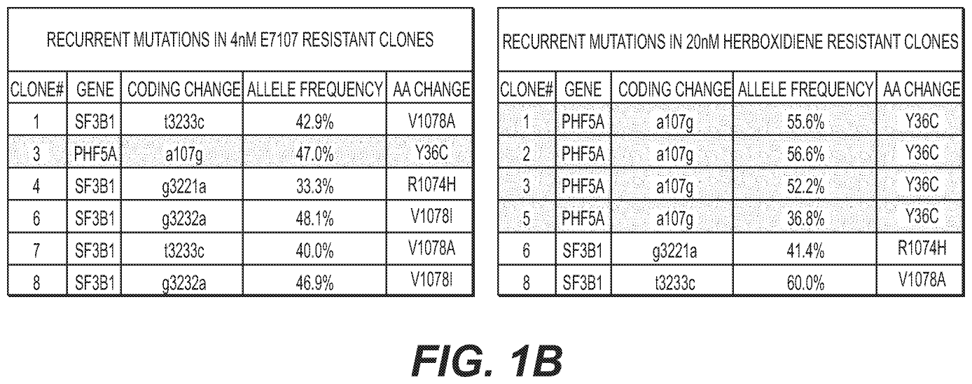

To further investigate the mechanism of splicing modulators targeting the SF3b complex, the possibility of resistant clone generation with lower stress levels of compound was explored via continuous administration of either lower dosage of E7107 (4 nM, approximately 3×GI50), a pladienolide derivative, or a less potent, structurally different splicing modulator, herboxidiene at 20 nM (approximately 3×GI50) in HCT116 cells (FIG. 1A). In contrast, previous approaches used stepwise induction of pladienolide B or E7107 doses up to 100 nM (approximately 130×GI50 in WiDR cells) to isolate resistant clones (Yokoi et al., The FEBS journal 278, 4870-80 (2011)). This approach could potentially mitigate off-target activity at high concentrations, as well as enhance the possibility to identify subtle but common mechanisms of splicing modulators. After two weeks of selection, six resistant clones from each treatment were expanded and subjected to whole exome sequencing (WXS) to identify candidate causal genes for resistance to splicing modulators. Compared to the parental line, totally about 11,000 single nucleotide variants (SNVs) and indels were identified with greater than 20% allele frequency. However, after cross-referencing with a curated splicing related gene list and focusing on genes affected in at least three individual clones, mutations in only two genes were consistently scored. Five out of six E7107 resistant clones and two of the six herboxidiene resistant clones carried mutations in SF3B1 (FIG. 1B), including the previously identified R1074H mutation and two novel mutations, V1078A and V1078I, strengthening the evidence that this region of SF3B1 is involved in splice modulator action. Interestingly, the remaining E7107 resistant clone and four herboxidiene resistant clones carried a Y36C mutation in PHF5A (FIG. 1B). All identified mutations in PHF5A and SF3B1 were further confirmed by targeted Sanger sequencing. In addition, Sanger sequencing revealed that one independent clone from 20 nM herboxidiene treatment appeared to be a pool of two individual populations, which harbored both PHF5A-Y36C and a novel K1071E mutation in SF3B1. While the apparent bias in mutation occurrences in either SF3B1 or PHF5A in the resistant clones (FIG. 1B) may implicate differences in how the pladienolide and herboxidiene scaffolds interact with the SF3b complex, these data suggest that both proteins are common cellular targets for splicing modulators.

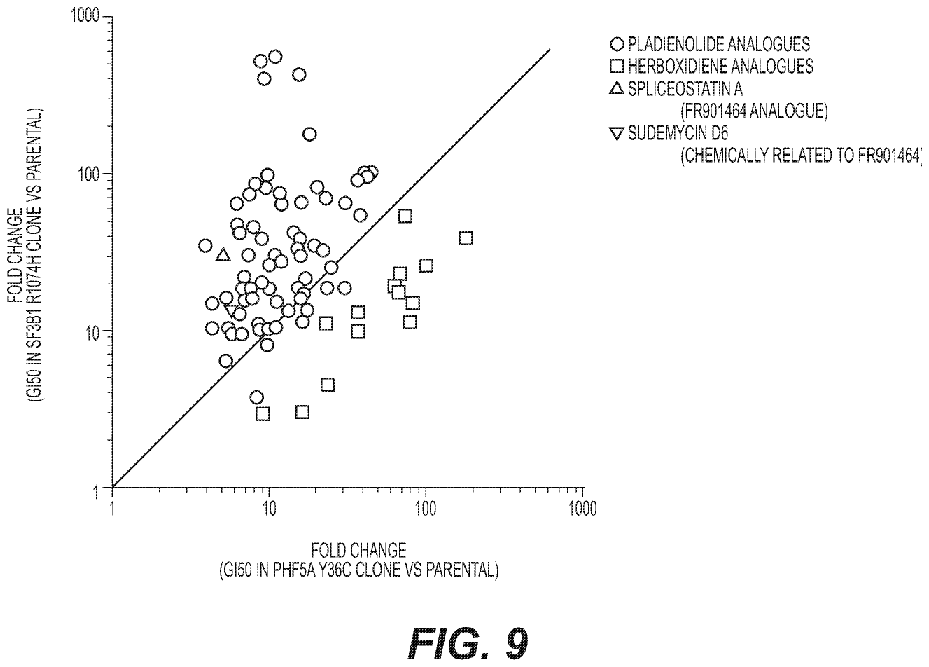

Growth inhibition profiling of the different resistant clones revealed that the SF3B1-R1074H mutation conferred the most robust resistance to E7107 whereas the PHF5A-Y36C and SF3B1-V1078 mutations were weaker (FIG. 1C). Interestingly, the SF3B1-R1074H mutation also conferred better resistance to spliceostatin A and sudemycin D6, both chemically related to FR901464, which is structurally different from pladienolides (FIGS. 1D and 1E). In contrast, the PHF5A-Y36C mutation rendered more resistance in response to herboxidiene treatment (FIG. 1F), in line with the higher percentage of clones harboring this mutation after herboxidiene selection (FIG. 1B). Mutations in SF3B1 or PHF5A did not affect the cell lines sensitivity to bortezomib, a pan-cytotoxic proteasome inhibitor, highlighting the specificity of the mutations toward splicing modulators (FIG. 1G). To validate the apparent preference for different scaffolds, CTG profiling to additional compounds was expanded and the GI50 shift in the SF3B1 R1074H clone was directly compared over the parental line versus the GI50 shift in the PHF5A Y36C clone. Both resistant mutations conferred resistance to all examined splicing modulators. Also, compounds appeared to cluster based on their scaffold, with PHF5A Y36C showing better resistance to the herboxidiene analogues and SF3B1 R1074H showing better resistance to the pladienolide and spliceostatin analogues (FIG. 9).

2.2. PHF5A-Y36C does not Affect Basal Cellular Functions but Confers Resistance to Splicing Modulators

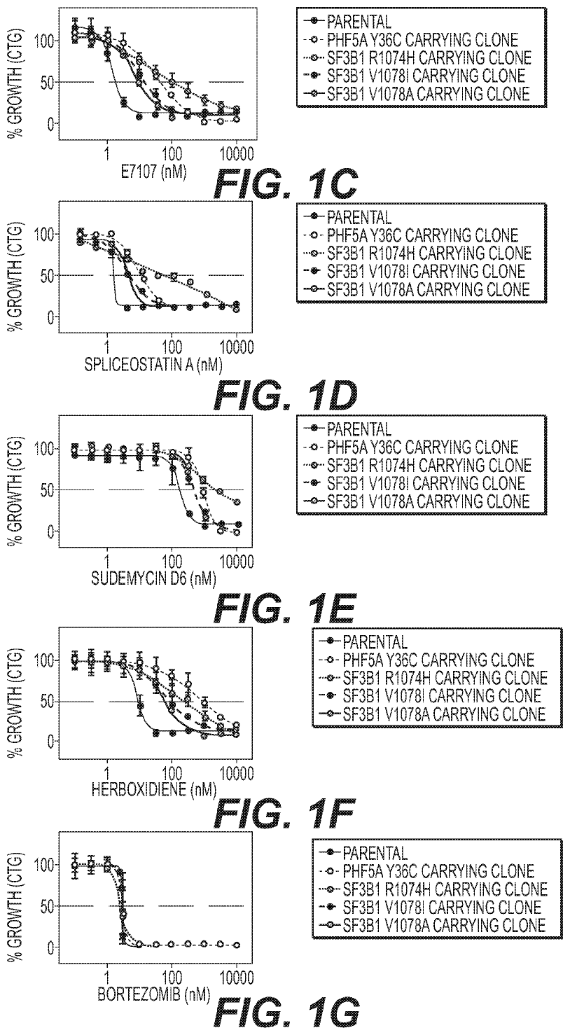

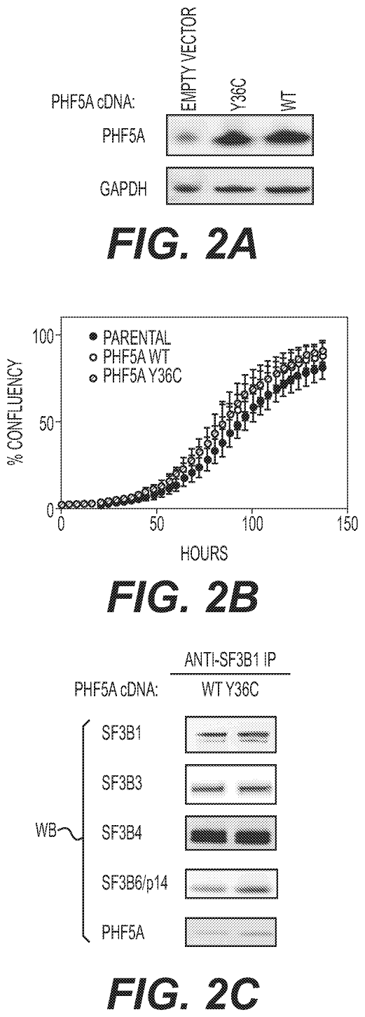

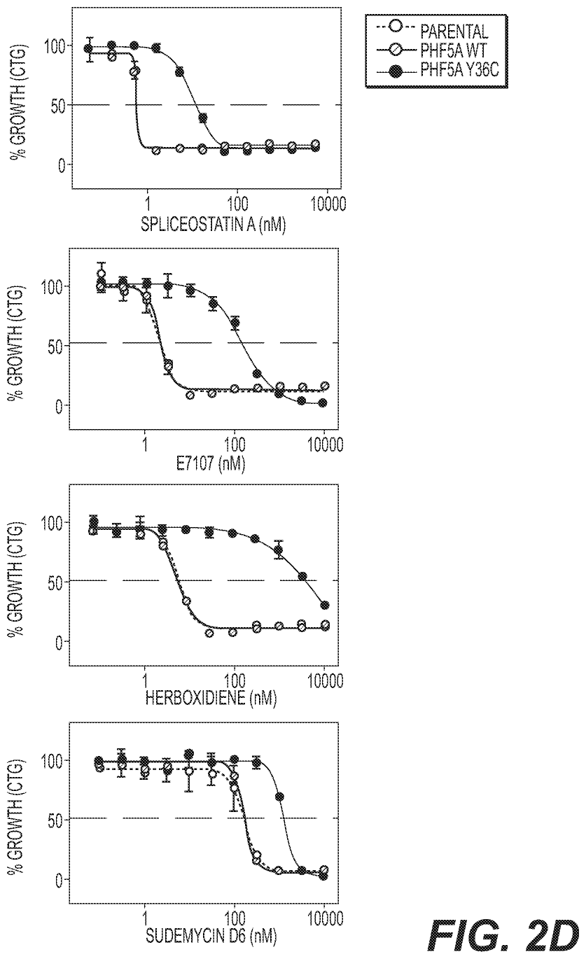

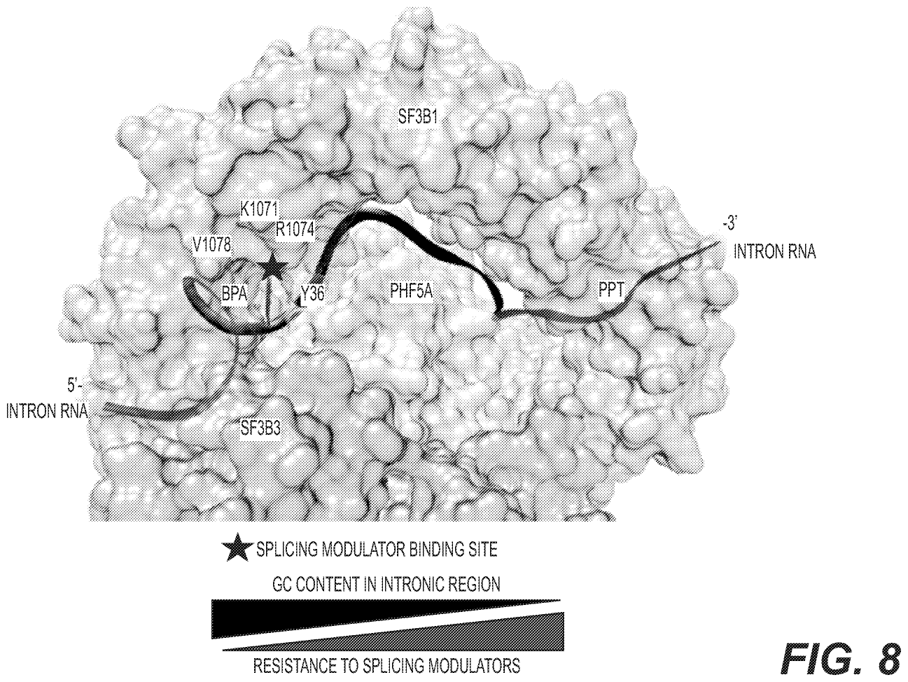

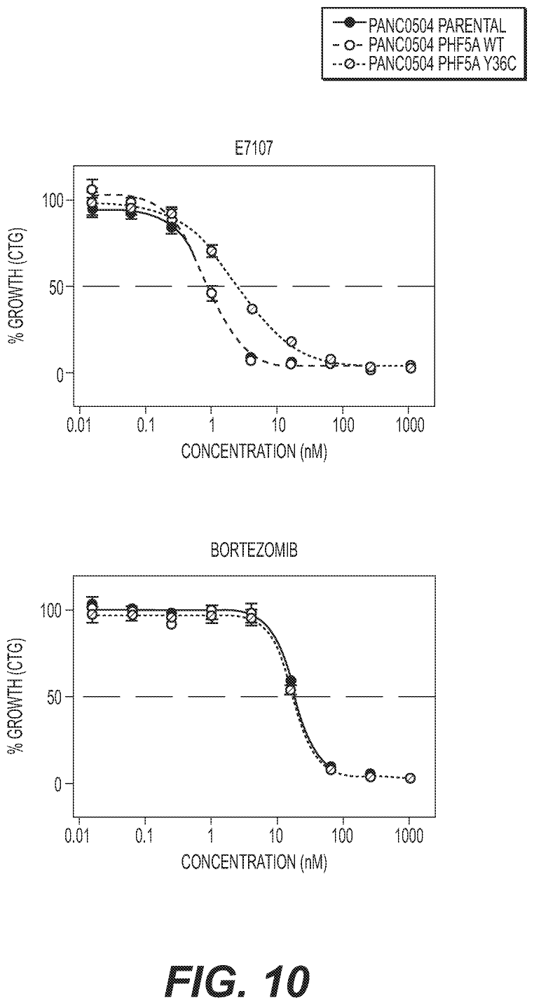

To further validate PHF5A-Y36C as a mechanism underlying resistance to splicing modulation, either wild-type (WT) PHF5A or Y36C PHF5A at similar levels in the parental HCT116 cell line were expressed (FIG. 2A). Despite the sequence conservation of this tyrosine residue through evolution (van Roon et al., Proceedings of the National Academy of Sciences of the United States of America 105, 9621-6 (2008)), expression of either PHF5A-WT or Y36C has no apparent effect on cell growth (FIG. 2B), localization of SF3B1 protein or formation of nuclear speckles. Given that PHF5A is one of seven proteins in the SF3b complex, whether the mutation could disrupt interactions with any of the core components and alter the overall composition of the complex was examined. Immunoprecipitated (IP'ed) samples by anti-SF3B1 antibodies from WT and mutant cell lines were subjected to western blot and mass-spectrometry analysis to qualitatively assess their composition (FIG. 2C). No significant differences in the overall composition of the complexes containing WT or Y36C PHF5A was observed, suggesting that aside from this mutation they are otherwise intact and functional. Whole-transcriptome RNA-seq analysis confirmed that expression of PHF5A-Y36C accounted for approximately 92% of the total PHF5A mRNA in the engineered cell line but had minimal effects on global splicing or gene expression when compared to WT (FIG. 8). Whereas parental cells and cells expressing WT PHF5A were sensitive to splicing modulator treatment, expression of PHF5A-Y36C conferred resistance to a panel of splicing modulators (FIG. 2D), phenocopying the spontaneous PHF5A Y36C resistant clones (FIG. 1C-1F). This resistance phenotype appears to be general as it was also observed when PHF5A-Y36C was introduced to another cell line (FIG. 10).

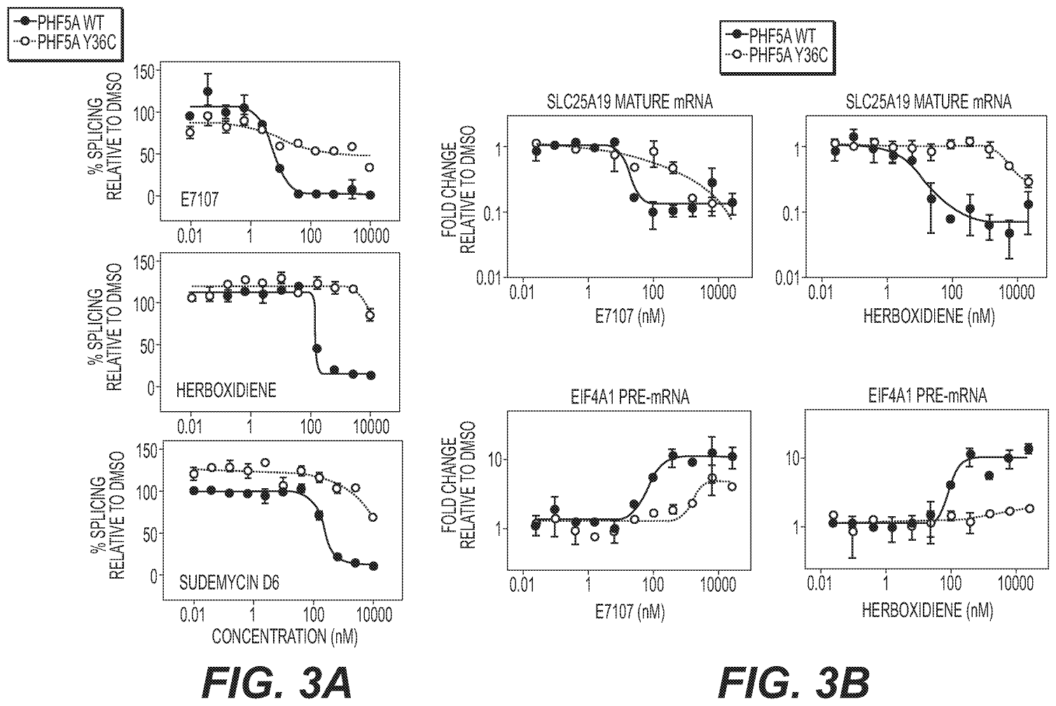

The behavior of the PHF5A-Y36C mutation at the biochemical level was next examined. Consistent with the cellular data (FIG. 2D), in vitro splicing assays with an exogenous pre-mRNA substrate showed that the Y36C mutant protected against the inhibition by splicing modulators of different scaffolds (FIG. 3A). To validate whether similar levels of protection are also present in vivo, quantitative real-time PCR analysis was used to assay the splicing of two endogenous pharmacodynamic marker genes which were used previously in the Phase I clinical trial of E7107 (Eskens et al., Clinical Cancer Research, 19, 6296-304 (2013)) (FIG. 3B). In agreement with the effect observed in in vitro splicing assays, Y36C mutation also reduced the inhibition on the production of spliced, mature SLC25A19 mRNAs and the accumulation of unspliced, immature EIF4A1 pre-mRNA elicited by splicing modulators (FIG. 3B). It appears that PHF5A-Y36C protects against splicing modulator induced mis-splicing.

### 2.3. PHF5A-Y36C Alters E7107 Induced Aberrant Splicing at a Global Level

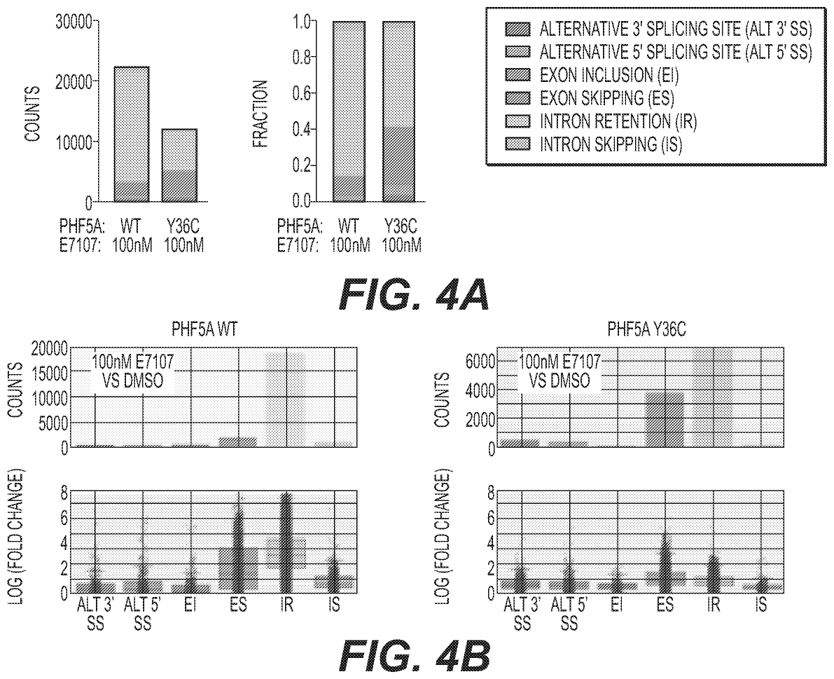

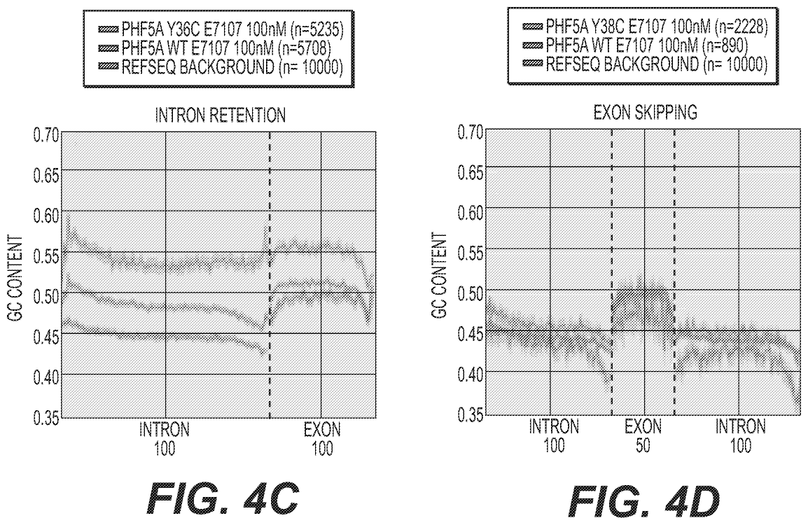

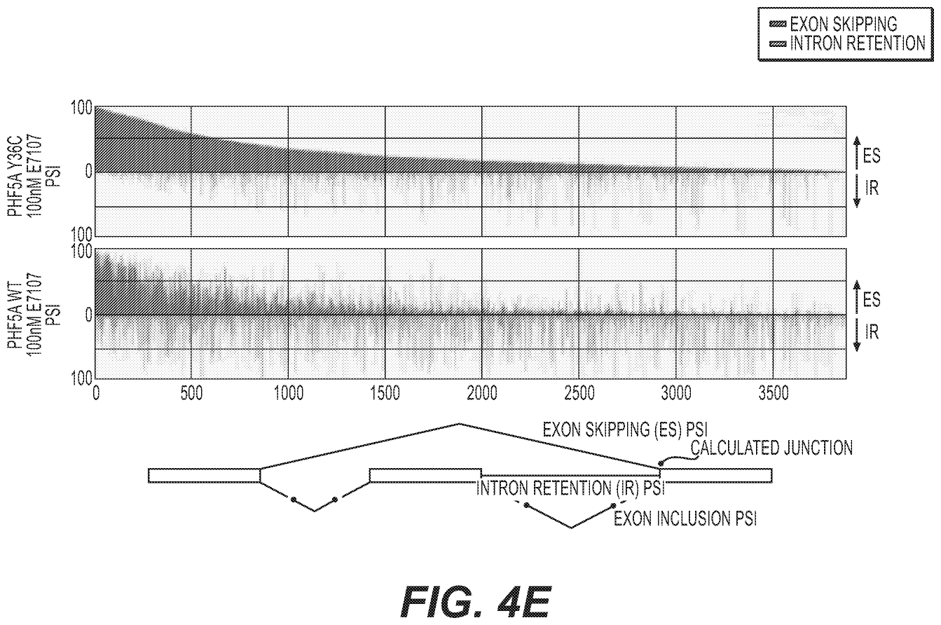

To examine how global splicing is affected by splicing modulators, whole transcriptome RNA-seq analysis was applied in both WT and Y36C PHF5A expressing cells treated with 100 nM E7107. Unsupervised clustering based on gene expression and principal component analysis of splicing junction usage confirmed that the Y36C cells treated with E7107 clustered away from their WT counterpart but near the DMSO treated controls, suggesting that the Y36C mutation weakened E7107 activity. Detailed differential splicing analysis further unveiled the quantitative and qualitative effects imposed by the Y36C mutation (FIGS. 4A and 4B). Specifically, compared to the respective DMSO treated controls, intron-retention (IR) events were predominant in WT cells treated with E7107 as measured by both the number of events and average fold change (FIGS. 4A and 4B left panel). Consistent with the protective effect of Y36C, the overall amount of IR events and their average fold change were greatly reduced in the mutant cells treated with E7107 (FIGS. 4A and 4B right panel). Surprisingly, the number of compound induced exon-skipping (ES) events was increased in the mutant cells compared to WT upon E7107 treatment (FIGS. 4A and 4B), suggesting that PHF5A-Y36C-mediated resistance to splicing inhibition involves a differential response at the global level.

The regulation of IR and ES events is known to be associated with exon/intron length and nucleotide content, as well as with specific chromatin marks (Naftelberg et al., Annual review of biochemistry 84, 165-98 (2015). Particularly, a differential in GC content between neighboring introns and exons may have evolved as recognition signals for the splicing machinery (Amit et al., Annual review of biochemistry 84, 165-98 (2015)). Therefore, this experiment sought to examine whether intronic GC content might also affect splice site recognition in PHF5A-WT or Y36C cells under splicing inhibition (FIGS. 4C and 4D). In WT cells, E7107 induced IR introns harbor higher GC content and less differential with the downstream exons as compared to the randomly selected background introns (FIG. 4C). Interestingly, IR introns/exons in PHF5A Y36C cells treated with E7107 displayed much higher GC composition and minimal differential between affected introns and exons as compared to its WT counterpart (FIG. 4C). In contrast, whereas ES junctions in compound treated WT cells showed lower GC composition than the background, ES junctions in Y36C cells treated with E7107 presented with higher GC content (FIG. 4D). In aggregate, these data suggest that intron/exon GC content may contribute to Y36C-mediated interference of splicing modulation.

Intriguingly, the intron/exon GC contents of IR events in WT cells (FIG. 4C) are comparable to those of ES events in Y36C cells (FIG. 4D). In addition, E7107 treatment induced more ES events but fewer IR events in PHF5A-Y36C cells (FIGS. 4A and 4B). Thus, it was hypothesized that some of these ES related introns from the Y36C cells might be switched to IR in the WT cells under the same E7107 treatment. To this end, the percentage (percent spliced in, PSI) of the individual 3′ intron-exon junction usage for these ES events in both PHF5A WT and Y36C were calculated. Theoretically, the outcome of these 3′ junctions would be either ES, IR, or exon inclusion (for scheme of the calculation, see FIG. 4E and Methods). Consistent with the ES/IR switch hypothesis, 2470 out of these 3883 Y36C related ES junctions (˜64%) showed reduced ES PSI and increase IR PSI in the WT cells treated with E7107 (FIG. 4E). This provided further evidence at the global level that PHF5A Y36C could weaken the activity of splicing inhibitors by modulating the usages of specific intron-exon junctions both quantitatively and qualitatively, utilizing the evolutionarily developed relative GC content of the neighboring introns/exons (Amit et al., Annual review of biochemistry 84, 165-98 (2015)).

### 2.4. The IR/ES Switch of MCL1 is Altered by PHF5A-Y36C in the Presence of E7107

Despite differences in the number of splicing events elicited by E7107, the overall numbers of affected genes from WT or Y36 cells were comparable and shared a large overlap. Gene Set Enrichment Analysis (GSEA) also identified candidate genes linked to pathways in either WT or Y36C specific genes. To validate the global differential splicing analyses, which revealed an IR/ES switch by splicing modulators in PHF5A-Y36C cells, genes which were associated with significant IR events in WT cells treated with E7107 as comparing to DMSO controls but were linked to significant ES events in Y36C under compound treatment were evaluated. A large number of genes such as MCL1, CDC25B, RBM5 and CDK10 were among the group, and individual sashimi plots validated the differential in splicing behavior between WT and Y36C cells treated with E7107 (FIG. 5A). MCL1 exists as two isoforms, MCL1-L and MCL1-S and was previously reported as a major target for splicing modulators such as meayamycin B (Gao and Koide ACS chemical biology 8, 895-900 (2013); Gao et al., Scientific reports 4, 6098 (2014) and sudemycin D1 (Xargay-Torrent et al., Oncotarget 6, 22734-49 (2015)). Interestingly, the second intron of MCL1 harbors a low (38%) GC content compared to the GC-rich (51%) upstream intron. Sashimi plots of the MCL1 RNA-seq data confirmed that in DMSO treated control samples both ES and IR events occurred at very low levels in WT and Y36C cells, resulting in dominant production of the canonical MCL1-L form (FIG. 5A). Upon E7107 treatment, IR was the dominant event observed in WT cells. In contrast, upon PHF5A Y36C expression, the effect of E7107 was largely altered, and mainly ES events were observed yielding the MCL1-S form (FIG. 5A).

Next, MCL1 was utilized as a biomarker to expand the analysis of the ES/IR switch to additional splicing modulators of different scaffolds and multiple dosages. Taqman gene expression not only confirmed the RNA-seq analysis but also revealed a correlation between the potency of splicing modulators and the relative rates of induction for ES and IR events. Specifically, in PHF5A WT cells, the more potent spliceostatin A (GI50=0.76 nM in HCT116) led to similar kinetics for dose-dependent induction of MCL1 ES and IR events, whereas the slightly less potent E7107 (GI50=1.5 nM in HCT116) presented with “earlier” induction of MCL1 ES events than IR events at lower doses. The weaker herboxidiene (GI50=7.6 nM in HCT116) showed an even more pronounced effect, and finally the IR events were not observed with the weakest compound tested, sudemycin D6 (GI50=149 nM in HCT116) (FIG. 5B left panels). These data strengthened the observation that the low GC containing intron 2 of MCL1 was more resistant to splicing inhibition than the higher GC containing intron 1 in the same gene. Expression of the PHF5A Y36C mutation delayed or blocked the onset of the MCL1 IR events in the presence of these splicing modulators (FIG. 5B right panels). Interestingly, MCL1-S production, representing ES events, was enhanced to a higher level in PHF5A-Y36C cells compared to WT upon increasing dosage of E7107 (FIG. 5b second row). Taken together, these data confirmed the observation that PHF5A Y36C controlled the switch between compound induced IR events and ES events.

### 2.5. Crystal Structure of Human PHF5A, the Core of the SF3b Complex

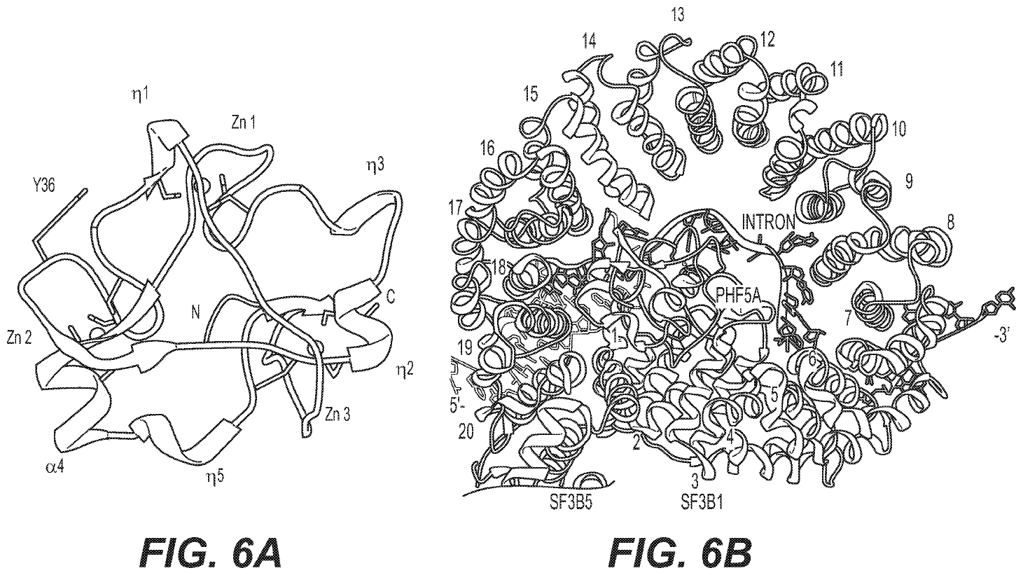

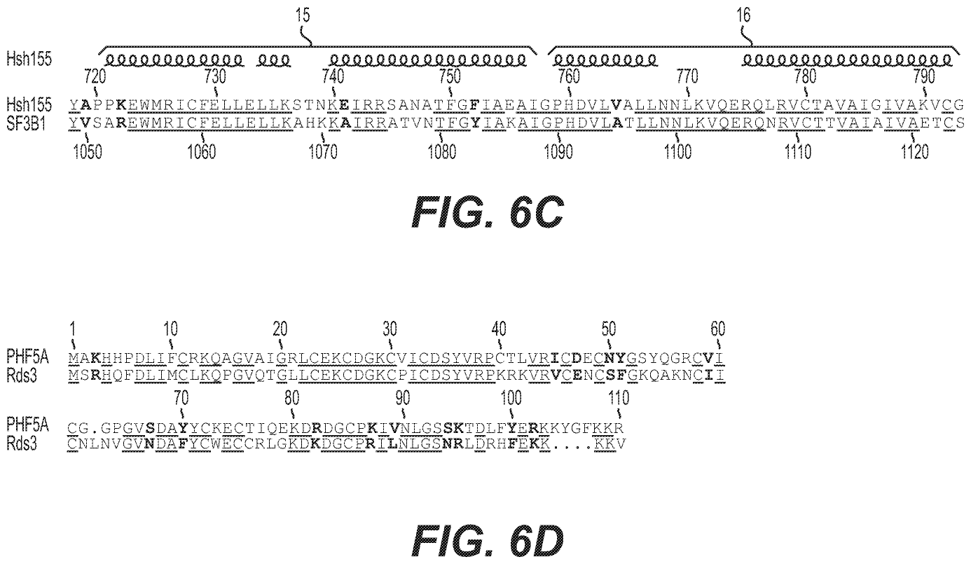

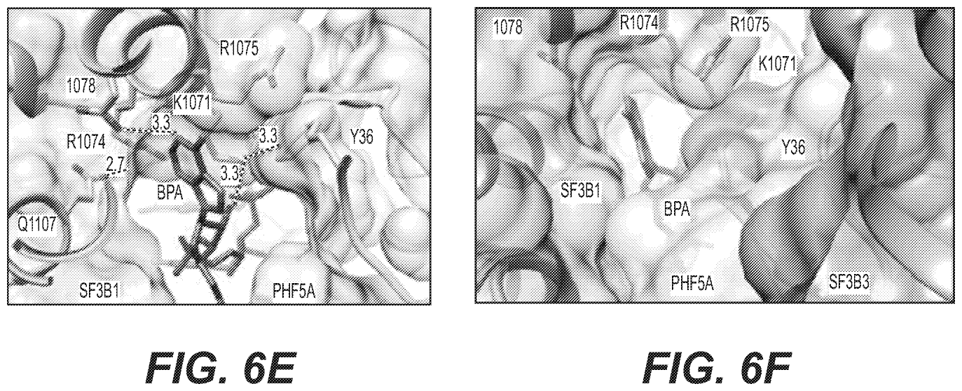

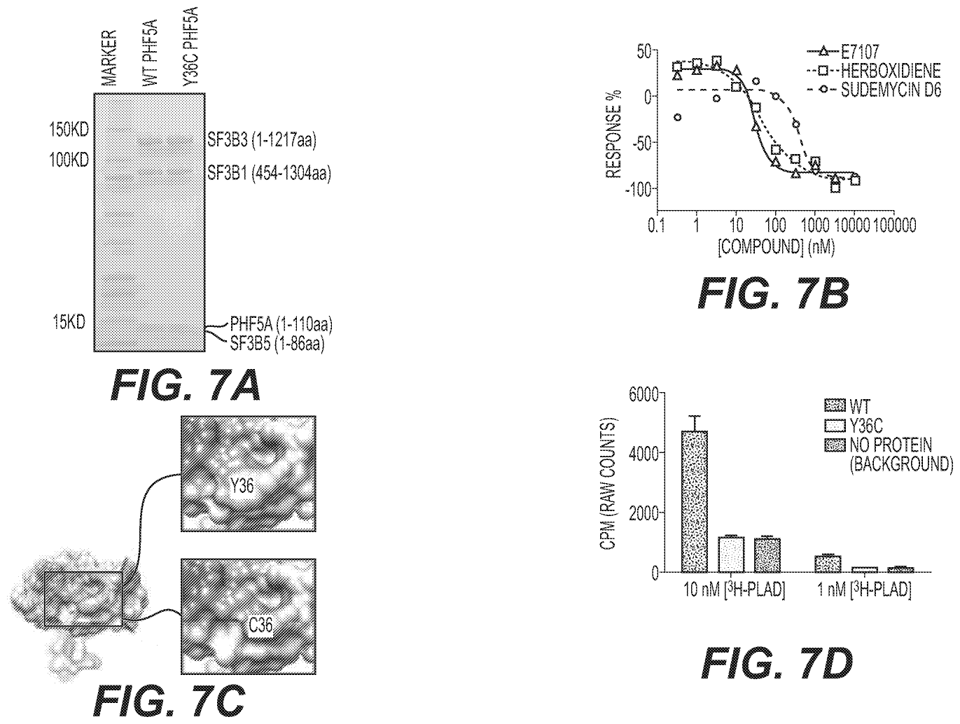

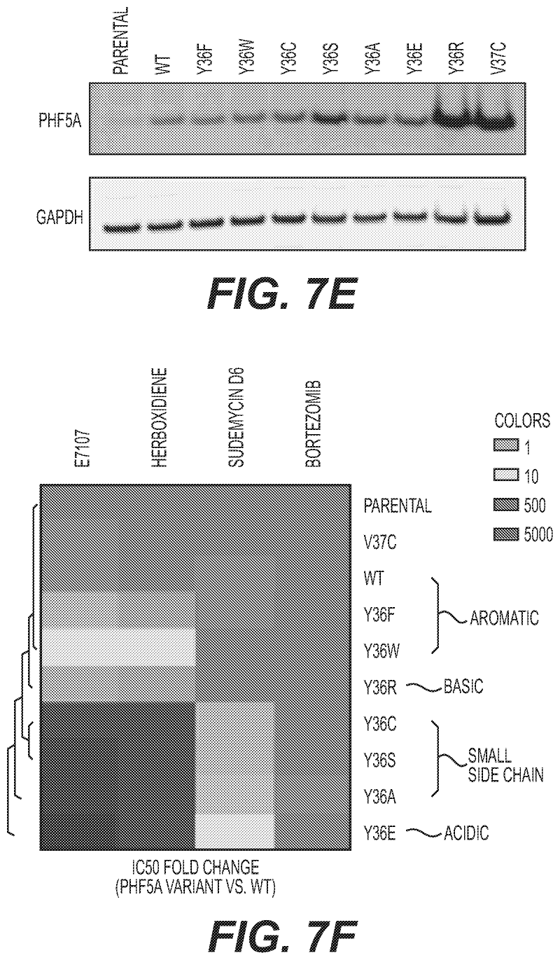

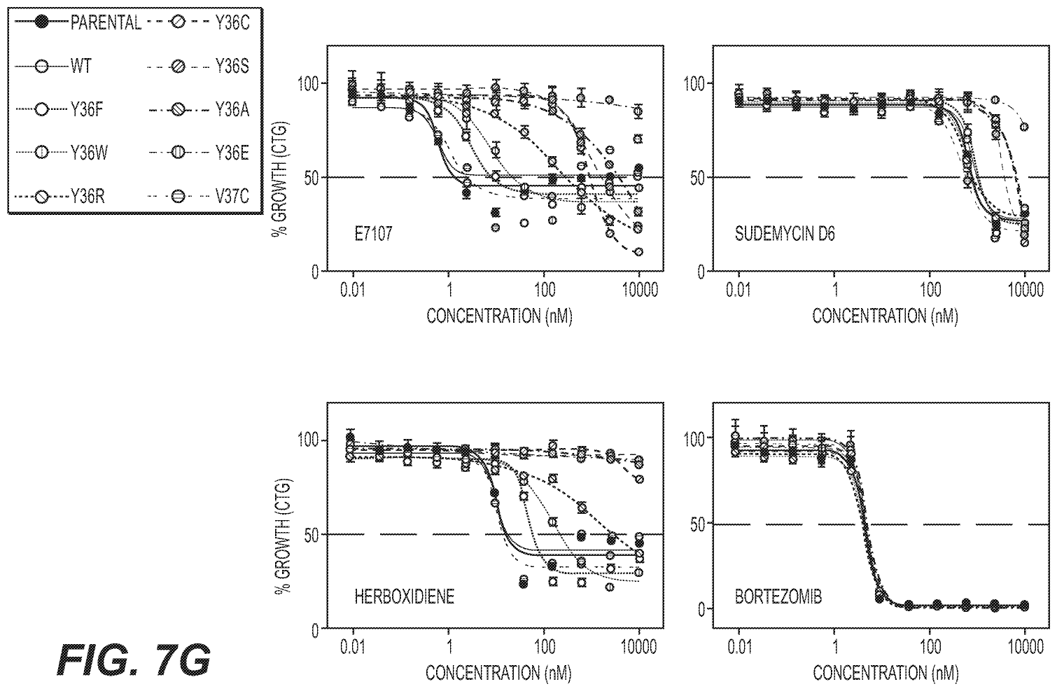

Given that Y36C PHF5A has no effect on basal splicing but plays a role in hindering and altering splicing modulators' effect on RNA splicing, the role of PHF5A in the context of the three dimensional structure was investigated. The WT protein determined the crystal structure at 1.8 Å resolution was purified. The final model contains residues 2-93 out of 110 total. PHF5A forms a mushroom-like structure with a triangular shaped cap and a stem composed of antiparallel strands from the N and C termini (FIG. 6D). The cap is formed by a left-handed, triangular, deep trefoil knot containing three zinc ions and 5 CXXC motifs, which are permuted between the zinc fingers. PHF5A contains 13 Cys residues and 12 of these coordinate 3 zinc ions in tetrahedral geometry. The remaining cysteine was mutated to serine (C40S) to enhance soluble protein expression. Interestingly, PHF5A incorporates three different types of zinc finger. Zinc finger 1 (ZnF1) folds into a gag knuckle and has C4 coordination from the first and fourth CXXC motifs. The first of these has a short helical turn (η1) while the fourth has a zinc knuckle (Krishna et al., Nucleic acids research 31, 532-50 (2003)). Zinc finger 2 (ZnF2) is formed by the second and fifth CXXC motifs. The first of these motifs is a zinc knuckle and the second comes from helix-α4 and therefore resembles the treble clef GATA-like zinc finger18. Zinc finger 3 (ZnF3) is formed by the third CXXC motif from helix-η2 and two individual cysteines from the loops connecting the first and the last beta strands of the mushroom stem. This third zinc finger resembles an interrupted classical ββα finger with a short helix (van Roon et al., Proceedings of the National Academy of Sciences of the United States of America 105, 9621-6 (2008); Krishna et al., Nucleic acids research 31, 532-50 (2003)). Given the location of PHF5A-Y36 on the surface near the second zinc finger, and the evidence that it does not alter any tested cellular activities, it is predicted that mutation to Cys would have minimal effect on the overall fold but rather act locally altering the surface topology (FIG. 7C).

While classified as a PHD finger, PHF5A has low sequence homology with other PHD fingers and differs from the canonical fold. A high level of sequence identity across diverse eukaryotic organisms shows its unique trefoil knot topology is likely to be conserved (FIG. 3D). At the same time, PHF5A has very low sequence identity when compared to other sequences within the same organism, suggesting a unique biological role in the cell. However, proteins with low sequence identity can still share similar three dimensional structures and have similar function. To explore this possibility, the structure was compared to all other available structures in the PDB and found only one other protein with similar fold, Rds3, a PHF5A homolog from yeast (Holm and Rosenstrom, Nucleic acids research 38, W545-9 (2010)). The Rds3 structure was solved by NMR, containing 80 residues and unstructured coils at the N- and C-termini van Roon et al., Proceedings of the National Academy of Sciences of the United States of America 105, 9621-6 (2008)). It also has three zinc fingers and the same trefoil knot fold (Z-scores 12.6 and RMSD 2.2 Å) (Holm and Rosenstrom, Nucleic acids research 38, W545-9 (2010)).

The full-length Rds3 protein was recently observed in the cryo-EM structure of the spliceosome Bact complex at a resolution range of 3.0-3.5 Å15. This structure shows that Rds3/PHF5A is a central scaffolding protein, interacting with Hsh155/SF3B1, Rse1/SF3B3, Ysf3/SF3B5, U2 snRNA and the intron RNA (FIG. 6B). Here, the SF3B1 HEAT repeats (HR) form a right-handed superhelical spiral of one complete turn forming a central ellipsoid cavity of approximately 34×39 Å (FIG. 6B). PHF5A nestles into this cavity forming extensive contacts along its sides with HR 2-3, 6, 15, and 17-20 (FIG. 6B). Of 110 total residues in PHF5A, 28 are forming contacts with SF3B1 burying 19% (1337 Å2) of surface area and a high degree of sequence conservation between the two interfaces. The C-terminal HR-20 helix and N-terminal helix of SF3B5 form a parallel helix-helix interaction that completes the superhelical turn while forming additional interactions with PHF5A (residues F6-L12) (FIG. 6B). SF3B3 sits along the top face of the SF3B1-PHF5A complex forming contacts with both, while the intronic RNA sits along the bottom face of the complex. Most of these interactions are to the phosphodiester backbone, as evidenced by complementary electropositive surface.

Superimposing the yeast and human PHF5A structures reveals structural differences at only two regions, which both form interactions with the intron RNA. The last helix (G93-R110) of the C-terminus, which is missing in the PHF5A crystal structure, contains conserved basic residues located between HR-2 from SF3B1 and the intron-U2 RNA duplex. These basic residues form multiple contacts to the intron nucleotides (+1-CACAUU) downstream of BPA (position 0). A minor difference is at the helix (η2)-loop-helix (η3) (from N50-R57) near ZnF3 where it has lower sequence conservation and also adopts multiple conformations in the Rds3 solution structure, suggesting this part of the molecule might be flexible. This region is making contact to two nucleotides (+9-AU) from the intron and the flexibility could accommodate conformations of different intronic RNAs.

### 2.6. Structural Analysis of Resistant Mutations in PHF5A and SF3B1

Recently, several cryo-EM structures have provided snapshots of the pre-catalytic and catalytic steps in the splicing reaction. The SF3b complex was only observed in the pre-catalytic Bact complex (Yan et al., Science 353, 904-11 (2016)). In the next step, rearrangements occur triggering dissociation of the SF3b complex and formation of the C complex, in which the phosphodiester bond has been made between the 2′-OH of the BPA and the 3′ phosphate of guanosine at the 5′-splice site (Folco et al., Genes & development 25, 440-4 (2011); Galej et al., Nature 537, 197-201 (2016); Wan et al., Science 353, 895-904 (2016)). Strikingly, the yeast Bact complex cryo-EM structure shows that the interface between PHF5A and SF3B1 is where the branchpoint adenosine (BPA) binds (FIG. 6E). These proteins from Sf3b complex apparently shield the reactive group from premature nucleophilic attack. Indeed, in this model, PHF5A-Y36 forms direct contact with the BPA, clearly implicating PHF5A in branchpoint recognition. This specialized biological role may explain its high sequence conservation and lack of any other apparent counterparts in the cell, which is consistent with previous finding of its roles in splicing regulation and splicing modulator sensitivity in glioblastoma stem cells (Hubert et al., Genes & development 27, 1032-45 (2013)). The HEAT repeats of SF3B1 that define this binding pocket (HR15-17) are also highly conserved (FIG. 6C). Interestingly, the resistance mutations identified in this study, PHF5A-Y36C, SF3B1-K1071E, SF3B1-V1078A/I, and previously reported SF3B1-R1074H, all cluster around this pocket (FIGS. 6E and 6F). Moreover, cross linking data show that these splicing modulators interact directly with SF3B1 and SF3B3 (Kotake et al., Nature chemical biology 3, 570-5 (2007); Hasegawa et al., ACS chemical biology 6, 229-33 (2011)), which sits immediately above this pocket (FIG. 6F). These striking coincidences provide evidence that this BPA binding pocket is also the region where splicing modulators bind. While conferring resistance, remarkably these mutations are not detrimental to basal splicing despite their proximity to the BPA. Detailed analysis shows that SF3B1-K1071 is a conserved residue (FIG. 6C) and forms H-bonds with the 2′-hydroxyl of the BPA ribose sugar and also with the hydroxyl of PHF5A-Y36, which helps to position and orient these residues at the interface (FIG. 6E). Since mutation of either of these residues results in resistance, this interaction is likely involved in modulator binding. PHF5A-Y36 also forms extensive van der Waals interaction with another conserved residue, SF3B1-R1075, which also helps orient this sidechain and alter the binding pocket. Based on the Y36C model, the mutation does not cause a significant change to the electrostatic surface but does alter the surface topology (FIG. 7C). The loss in affinity suggests the aromatic sidechain at this position is involved in splice modulator binding. SF3B1-R1074H is located at the base of this binding pocket (FIG. 6E). It does not make any direct interactions with RNA or PHF5A, but mutation would alter the shape of the binding pocket and could affect compound binding but not BPA interaction (FIGS. 6E and 6F). SF3B1-V1078A/I is near the top of this pocket and not conserved between yeast and human (FIG. 6C). In yeast, this residue forms an H-bond to the BPA adenosine, but in humans this residue is likely to result in a relatively subtle change and indeed confers the least amount of overall resistance.

### 2.7. PHF5A-Y36C Reduces the Binding Affinity of Splicing Modulators

In order to demonstrate the splicing modulator binding site is at the interface composed by SF3B1, PHF5A and SF3B3, a recombinant protein complex based on the yeast B^{act} cryo-EM structure was engineered (Yan et al., Science 353, 904-11 (2016)). By co-expressing these three proteins with SF3B5, it was possible to reconstitute a stable 250 kDa complex that could be purified in two-steps (FIG. 7A). To validate this recombinant complex can recapitulate a functional modulator binding site, it was captured on scintillation proximity assay (SPA) beads and probed its interaction with a ³H-labeled pladienolide analogue (Kotake et al., Nature chemical biology 3, 570-5 (2007)). SPA assays revealed ³H-labeled pladienolide probe bound to the complex and other non-radioactive splicing modulators were able to compete off the bound probe, demonstrating the specificity of the interaction (FIG. 7B). In this competition assay, reduced signal from titrating non-radioactive modulators reveals the relative affinity of these three compounds to the complex compared to the pladienolide-like analogue and is consistent to the potency and rank ordering seen in the IVS assay (FIG. 3A) and the cellular assay (FIG. 2D). This validates that these four proteins reconstitute a functional binding site for splicing modulators.

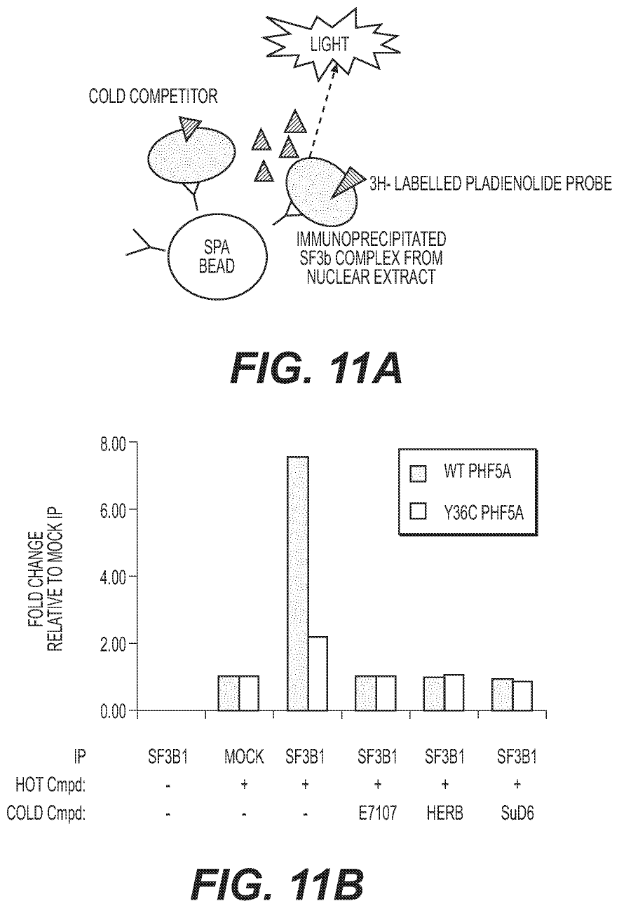

Next, the corresponding complex containing PHF5A-Y36C was generated to inspect whether the observed resistance mutation is a result of reduced binding between splicing modulator(s) and the SF3b complex. Purified PHF5A-Y36C recombinant complex was captured on the SPA beads and the same ³H-labeled tracer compound Kotake et al., Nature chemical biology 3, 570-5 (2007)) was used to probe the interaction at two different concentration, 10 nM and 1 nM. SPA assay reveals that an approximate 5 fold induction of the 10 nM ³H-labeled probe binding to the WT PHF5A containing complex over background, whereas the binding to the PHF5A-Y36C complex was equal to background. This demonstrates that the single Y36C mutation is sufficient to reduce modulator binding significantly (FIG. 7D) and suggests Y36 interacts with modulators. The reduced affinity was also observed in the IP'ed SF3b complex from PHF5A-Y36C cell nuclear lysates, confirming that this mutation is able to decrease modulator binding in a physiological relevant protein complex as well (FIG. 11).

### 3. Discussion

Spliceosomes undergo multiple ATP-dependent conformational changes involving a number of snRNPs, and this dynamic complexity makes it challenging to determine where and when splicing modulators bind. Previous photocrosslinking studies with pladienolide and herboxidiene analogues narrowed down the interaction point to the SF3b complex, one of the subunits of the U2 snRNP, specifically to the individual proteins SF3B3 and SF3B1 (Kotake et al., Nature chemical biology 3, 570-5 (2007); Hasegawa et al., ACS chemical biology 6, 229-33 (2011)). The resistant mutation SF3B1-R1074H generated under high doses of pladienolide B and E7107 provided further evidence that SF3B1 is involved in compound binding (Yokoi et al., The FEBS journal 278, 4870-80 (2011)). By applying a genomic resistance mapping approach with low doses of E7107 and herboxidiene novel resistance mutations were elicited. This allows the splicing modulator binding pocket to be assessed and potentially to further refine and account for the mechanism of action among certain introns. A series of mutations, Y36C in PHF5A, V1078A/I, K1071E and the previously identified R1074H (Id.) in SF3B1 were uncovered. Together with the photocrosslinking data (Kotake et al., Nature chemical biology 3, 570-5 (2007); Hasegawa et al., ACS chemical biology 6, 229-33 (2011)), the modulator binding pocket was pinpointed to the interface between PHF5A, SF3B1, and SF3B3 (FIG. 8). The other two modulators, spliceostatin A and sudemycin D, also show resistance to the Y36C clone indicating that these compounds interact with this site as well (Kaida et al., Nature chemical biology 3, 576-83 (2007); Xargay-Torren et al., Oncotarget 6, 22734-49 (2015)). Indeed, the binding of splicing modulators to this common binding pocket was confirmed by reconstituting a functional 4-protein complex consisting of PHF5A, SF3B1, SF3B3 and SF3B5 (FIG. 7A). Furthermore, the single amino acid substitution of Y36C reduced the binding of the pladienolide probe to background levels, suggesting that the mechanism of resistance is due to the decreased affinity of splicing modulators to the binding pocket (FIG. 7C). Detailed site-directed mutagenesis of Y36 shows that both the aromatic ring and electrical charge at the Y36 residue are involved in the activity of splicing modulators (FIG. 7E-7G). Furthermore, mutations at Y36 revealed different levels of protection against these modulators with different scaffolds, indicating that these modulators may adopt slightly different poses within mode of interaction at this common binding pocket. Webb et al., have previously hypothesized several pharmacophore features for herboxidiene activity including a hydrophobic motif (a diene group) between C8 to C11 (Lagisetti et al., ACS chemical biology 9, 643-8 (2014)). Pladienolide and herboxidiene share this diene moiety, implying this may bind at the proximity of Y36.

Given the location of the resistance mutations around the BPA binding site, one possible model for the mechanism of action is that the splicing modulators are BPA competitive inhibitors (FIG. 8). This close proximity of splicing modulators binding pocket to the BPA is consistent with previous reports that both spliceostatins and pladienolides impair the canonical base pairing between U2 snRNA and pre-mRNA branch point region in the presence of heparin (Folco et al., Genes & development 25, 440-4 (2011); Corrionero et al., Genes & development 25, 445-59 (2011)). Corrionero et al showed that spliceostatin A prevents U2 snRNP from establishing canonical base-pairing between the pre-mRNA and U2 snRNA in the presence of heparin (5 mg/mL), which impedes U2 snRNP from complex A assembly on the pre-mRNA (Corrionero et al., Genes & development 25, 445-59 (2011)). In addition, the splicing modulators E7107 and pladienolide B were found to have a similar weakening effect on binding of U2 snRNP to pre-mRNA (Folco et al., Genes & development 25, 440-4 (2011)). In these studies, the excess of negatively charged heparin presumably serves to further weaken the interaction between U2 snRNA and the pre-mRNA by disrupting cooperative, but nonspecific, interactions that help tether them to the protein complex. Therefore, in the absence of heparin, splicing modulators may weaken but not completely disrupt the interaction between the U2 snRNA and pre-mRNA (Corrionero et al., Genes & development 25, 445-59 (2011)). Moreover, in vitro splicing reactions show that inhibition depends on the order of reagent addition, namely a compound must be added to the nuclear extracts prior to substrate and ATP or else the reaction will proceed normally (Folco et al., Genes & development 25, 440-4 (2011)). These data suggest that the compounds act on the U2 snRNP early in spliceosomal assembly before the ATP-dependent transition in which the substrate pre-mRNA is loaded. It also points to an irreversible commitment step that cannot be blocked once the U2 snRNP has assembled onto the pre-mRNA. Collectively, these observations led to a model where splicing modulators directly impact on the fidelity of SF3B1 branch site recognition with consequences on the 3′ splice site recognition (Corrionero et al., Genes & development 25, 445-59 (2011)). This competitive binding model immediately suggests several possible functional consequences that can be examined at the global splicing level. Specifically, weaker GC rich intron substrates would be easier to inhibit than stronger intron sequences and this differential could manifest itself through alterations in splicing preferences in the presence of different compounds.

Consistent with this model for inhibition, here a nonlinear dose response was observed in global splicing due to variations in individual intron “strength.” Splicing modulation is a global phenomenon, which impacts more than 200,000 introns in the human genome (Sakharkar et al., In silico biology 4, 387-93 (2004)). Despite several conserved features within introns and adjacent exons, regulation of individual introns during splicing is both diverse and complex. This variation and complexity means that small molecule inhibition will have differential effects on splice junction usage. Here, a protective mutation in PHF5A allowed the individual cellular responses of introns upon splicing modulation to be examined, which revealed transitions between intron retention (IR) and exon skipping (ES) events.

It has been proposed that during evolution, the generally shorter, low GC containing introns in lower eukaryotes evolved under two different routes (Amit et al., Cell reports 1, 543-56 (2012)) one group of introns remained short, but had markedly increased GC percentage and had less differential in term of GC composition compared to their neighboring exons. Due to the shorter length of these introns, they are more likely to be recognized by an intron-defined splicing mechanism. Interestingly, these introns appear to be more susceptible to intron-retention upon E7107 treatment. Also, it was observed that when the effect of E7107 was weakened in the presence of PHF5A Y36C mutation, the average GC compositions of IR events related introns were markedly higher with little to no differential from downstream exons (FIG. 4C). Given that the differential in GC composition between introns and surrounding exons might contribute to splicing machinery recognition, it is plausible to hypothesize that these kinds of introns are inherently more difficult for the splicing machinery to recognize, which in turn might make them easier to inhibit with splicing modulators. It has also been proposed that higher GC content around BPA may lead to a more stable secondary structure of the pre-mRNA, therefore it is also plausible that GC content may affect the effectiveness of competition between pre-mRNAs and splicing modulators via structural and spatial mechanisms (Zhang et al., BMC genomics 12, 90 (2011)).

In contrast, another group of introns maintained their low GC composition and large differential with adjacent exons during evolution, but underwent significant increases in length, which likely brought them out of the range of intron-defined splicing and converted them to an exon-defined splicing mechanism. Intriguingly, under E7107 treatment, introns associated with increased ES events are associated with lower GC composition and higher GC differential with the skipped exons (FIG. 4D). Similar to the observation in IR events, the GC content of compound induced ES introns in the presence of Y36C was also higher than that of the WT cells (FIG. 4D). A higher differential in GC composition between introns and exons has been linked to increased nucleosome occupancy and enrichment of SF3B1 association with the chromatin, which presumably primes these junctions for co-transcriptional splicing (Amit et al., Cell reports 1, 543-56 (2012); Kfir et al., Cell reports 11, 618-29 (2015)). Further characterization of the genomic structure of the junctions associated with ES events may yield additional insight into understanding of the complex link between transcription and splicing.

Here, it was observed that 2470 junctions can be switched between IR and ES upon E7107 treatment depending on the genotype of PHF5A strengthens the hypothesis that introns possess differential sensitivity to small molecule inhibitors (FIG. 4E). The fact that IR and ES events affect the same 3′ junction are not mutually exclusive further unveils the plasticity of splicing regulation and a fine-tuning mechanism of the usage of individual junctions. Specifically, these approximately 2470 junctions display intermediate sensitivity to splicing inhibition and are switchable between IR and ES events depending on the level of splicing inhibition. It is conceivable that in PHF5A WT cells, E7107 was efficient in competing with the canonical BPAs in these 2470 junctions and led to intron-retention events. However, upon PHF5A Y36C expression, as the association of the compound with the PHF5A-SF3B1 interface was weakened but not lost, E7107 would become less efficient in the competition with these junctions while maintaining its competence with the immediate upstream introns, which therefore induced more exon-skipping events. This is in contrast with other weaker, high GC content introns which can be readily retained with E7107 even in the presence of PHF5A Y36C mutation (FIG. 4A-4C). Interestingly, some of the aforementioned 3883 ES related junctions were not associated with increased IR events in the presence of WT PHF5A, suggesting that these junctions could be even stronger and more resistant to splicing modulation (FIG. 4E). Furthermore, E7107 only induced aberrant splicing in approximately 20,000 introns (FIG. 4A), suggesting the existence of even stronger introns which can withstand splicing modulation at this dosage, which is consistent with previous observation using splicing sensitive microarray that spliceostatin A only impacted on selective 3′ splice sites (Corrionero et al., Genes & development 25, 445-59 (2011)). Collectively, these differential sensitivities from cellular introns are consistent with the model that splicing modulators act as competitive BPA inhibitors, and are likely to result in the nonlinear response to differential dosages of splicing modulators. Interestingly, some of the junctions identified in this study are players in cell cycle regulation and RNA-binding, i.e. RBM5, which has been shown to be a functional group preferentially modulated by spliceostatin A (Corrionero et al., Genes & development 25, 445-59 (2011)). Given the frequent alterations surrounding the pathway in tumorigenesis, further analysis of how splicing machinery contributes to the regulation of normal and aberrant cell cycle regulation could provide an additional route to target cancer cells.

Phenotypic screening of small molecule libraries is a powerful way to identify potential drugs. However, cellular target identification for the screening hits has been an unremitting challenge. Several unbiased approaches have been developed to identify the cellular targets and mechanisms of action, including biochemical approaches such as affinity purification coupled with quantitative proteomics, genetic interaction approaches such as RNAi screening and domain focused CRISPR screens, and computational inference approaches (Shi et al., Nature biotechnology 33, 661-7 (2015); Schenone et al., Nature chemical biology 9, 232-40 (2013)). More recently, next-generation sequencing (NGS)-based genomic or transcriptomic profiling of phenotypically resistant cell populations has been used (Adams et al., ACS chemical biology 9, 2247-54 (2014); Korpal et al., Cancer discovery 3, 1030-43 (2013); Wacker et al., Nature chemical biology 8, 235-7 (2012)) to identify unique recurrent single nucleotide variations (SNVs) or expression alterations to illuminate potential cellular targets of compounds. Here, the method by screening structurally unrelated compounds at different low concentrations was further developed, in order to 1) mitigate the potential off-target activity at high concentrations, and 2) enhance the possibility to identify subtle but common mechanisms of chemical probes. This allowed multiple mutations/genes encoding proteins co-existing in the same complex to be uncovered. Interestingly, the finding of resistant mutations to PHF5A-Y36, SF3B1-V1078 and K1071, in addition to confirming the previously reported SF3B1-R1074, suggests the proximity of these residues to the action site of splicing modulators. The fact that corresponding amino acids of these residues in yeast were recently shown to form a pocket that accommodates the invariant adenosine in the BPS demonstrates that this genomic profiling strategy can provide faithful and informative insights into the action of candidate compounds. Hence, further expansion of the genomic profiling approach will likely offer a unique way to explore the MoA (mechanisms of action) for compounds using the “2-dimensional” genomic fingerprint dissection. This is particularly valuable when the protein structure and/or biochemical assays with purified proteins are not readily available as exemplified in this study by the complex and dynamic spliceosome.

In summary, PHF5A was identified as a node of interaction for small molecule splicing modulators. Structural analysis pinpointed a common binding site around the branch point adenosine binding pocket. Also, the results demonstrate how a single amino acid change on PHF5A Y36 weakened the inhibitory effect of splicing modulators and altered the global splicing pattern between exon-skipping events and intron-retention events.

SEQUENCE LISTING

| SEQ ID NO: | Sequence |
|---|---|
| 1 | Met Ala Lys Ile Ala Lys Thr His Glu Asp Ile Glu Ala Gln Ile Arg |
| | Glu Ile Gln Gly Lys Lys Ala Ala Leu Asp Glu Ala Gln Gly Val Gly |
| | Leu Asp Ser Thr Gly Tyr Tyr Asp Gln Glu Ile Tyr Gly Gly Ser Asp |
| | Ser Arg Phe Ala Gly Tyr Val Thr Ser Ile Ala Ala Thr Glu Leu Glu |
| | Asp Asp Asp Asp Asp Tyr Ser Ser Ser Thr Ser Leu Leu Gly Gln Lys |
| | Lys Pro Gly Tyr His Ala Pro Val Ala Leu Leu Asn Asp Ile Pro Gln |
| | Ser Thr Glu Gln Tyr Asp Pro Phe Ala Glu His Arg Pro Pro Lys Ile |
| | Ala Asp Arg Glu Asp Glu Tyr Lys Lys His Arg Arg Thr Met Ile Ile |
| | Ser Pro Glu Arg Leu Asp Pro Phe Ala Asp Gly Gly Lys Thr Pro Asp |
| | Pro Lys Met Asn Ala Arg Thr Tyr Met Asp Val Met Arg Glu Gln His |
| | Leu Thr Lys Glu Glu Arg Glu Ile Arg Gln Gln Leu Ala Glu Lys Ala |
| | Lys Ala Gly Glu Leu Lys Val Val Asn Gly Ala Ala Ala Ser Gln Pro |
| | Pro Ser Lys Arg Lys Arg Arg Trp Asp Gln Thr Ala Asp Gln Thr Pro |
| | Gly Ala Thr Pro Lys Lys Leu Ser Ser Trp Asp Gln Ala Glu Thr Pro |
| | Gly His Thr Pro Ser Leu Arg Trp Asp Glu Thr Pro Gly Arg Ala Lys |
| | Gly Ser Glu Thr Pro Gly Ala Thr Pro Gly Ser Lys Ile Trp Asp Pro |
| | Thr Pro Ser His Thr Pro Ala Gly Ala Ala Thr Pro Gly Arg Gly Asp |
| | Thr Pro Gly His Ala Thr Pro Gly His Gly Gly Ala Thr Ser Ser Ala |
| | Arg Lys Asn Arg Trp Asp Glu Thr Pro Lys Thr Glu Arg Asp Thr Pro |
| | Gly His Gly Ser Gly Trp Ala Glu Thr Pro Arg Thr Asp Arg Gly Gly |
| | Asp Ser Ile Gly Glu Thr Pro Thr Pro Gly Ala Ser Lys Arg Lys Ser |
| | Arg Trp Asp Glu Thr Pro Ala Ser Gln Met Gly Gly Ser Thr Pro Val |
| | Leu Thr Pro Gly Lys Thr Pro Ile Gly Thr Pro Ala Met Asn Met Ala |
| | Thr Pro Thr Pro Gly His Ile Met Ser Met Thr Pro Glu Gln Leu Gln |
| | Ala Trp Arg Trp Glu Arg Glu Ile Asp Glu Arg Asn Arg Pro Leu Ser |
| | Asp Glu Glu Leu Asp Ala Met Phe Pro Glu Gly Tyr Lys Val Leu Pro |
| | Pro Pro Ala Gly Tyr Val Pro Ile Arg Thr Pro Ala Arg Lys Leu Thr |
| | Ala Thr Pro Thr Pro Leu Gly Gly Met Thr Gly Phe His Met Gln Thr |
| | Glu Asp Arg Thr Met Lys Ser Val Asn Asp Gln Pro Ser Gly Asn Leu |
| | Pro Phe Leu Lys Pro Asp Asp Ile Gln Tyr Phe Asp Lys Leu Leu Val |
| | Asp Val Asp Glu Ser Thr Leu Ser Pro Glu Glu Gln Lys Glu Arg Lys |
| | Ile Met Lys Leu Leu Leu Lys Ile Lys Asn Gly Thr Pro Pro Met Arg |
| | Lys Ala Ala Leu Arg Gln Ile Thr Asp Lys Ala Arg Glu Phe Gly Ala |
| | Gly Pro Leu Phe Asn Gln Ile Leu Pro Leu Leu Met Ser Pro Thr Leu |
| | Glu Asp Gln Glu Arg His Leu Leu Val Lys Val Ile Asp Arg Ile Leu |
| | Tyr Lys Leu Asp Asp Leu Val Arg Pro Tyr Val His Lys Ile Leu Val |
| | Val Ile Glu Pro Leu Leu Ile Asp Glu Asp Tyr Tyr Ala Arg Val Glu |
| | Gly Arg Glu Ile Ile Ser Asn Leu Ala Lys Ala Ala Gly Leu Ala Thr |
| | Met Ile Ser Thr Met Arg Pro Asp Ile Asp Asn Met Asp Glu Tyr Val |
| | Arg Asn Thr Thr Ala Arg Ala Phe Ala Val Val Ala Ser Ala Leu Gly |
| | Ile Pro Ser Leu Leu Pro Phe Leu Lys Ala Val Cys Lys Ser Lys Lys |
| | Ser Trp Gln Ala Arg His Thr Gly Ile Lys Ile Val Gln Gln Ile Ala |
| | Ile Leu Met Gly Cys Ala Ile Leu Pro His Leu Arg Ser Leu Val Glu |
| | Ile Ile Glu His Gly Leu Val Asp Glu Gln Gln Lys Val Arg Thr Ile |
| | Ser Ala Leu Ala Ile Ala Ala Leu Ala Glu Ala Ala Thr Pro Tyr Gly |
| | Ile Glu Ser Phe Asp Ser Val Leu Lys Pro Leu Trp Lys Gly Ile Arg |
| | Gln His Arg Gly Lys Gly Leu Ala Ala Phe Leu Lys Ala Ile Gly Tyr |
| | Leu Ile Pro Leu Met Asp Ala Glu Tyr Ala Asn Tyr Tyr Thr Arg Glu |
| | Val Met Leu Ile Leu Ile Arg Glu Phe Gln Ser Pro Asp Glu Glu Met |
| | Lys Lys Ile Val Leu Lys Val Val Lys Gln Cys Cys Gly Thr Asp Gly |
| | Val Glu Ala Asn Tyr Ile Lys Thr Glu Ile Leu Pro Pro Phe Phe Lys |
| | His Phe Trp Gln His Arg Met Ala Leu Asp Arg Arg Asn Tyr Arg Gln |
| | Leu Val Asp Thr Thr Val Glu Leu Ala Asn Lys Val Gly Ala Ala Glu |
| | Ile Ile Ser Arg Ile Val Asp Asp Leu Lys Asp Glu Ala Glu Gln Tyr |
| | Arg Lys Met Val Met Glu Thr Ile Glu Lys Ile Met Gly Asn Leu Gly |
| | Ala Ala Asp Ile Asp His Lys Leu Glu Glu Gln Leu Ile Asp Gly Ile |
| | Leu Tyr Ala Phe Gln Glu Gln Thr Thr Glu Asp Ser Val Met Leu Asn |
| | Gly Phe Gly Thr Val Val Asn Ala Leu Gly Lys Arg Val Lys Pro Tyr |
| | Leu Pro Gln Ile Cys Gly Thr Val Leu Trp Arg Leu Asn Asn Lys Ser |
| | Ala Lys Val Arg Gln Gln Ala Ala Asp Leu Ile Ser Arg Thr Ala Val |
| | Val Met Lys Thr Cys Gln Glu Glu Lys Leu Met Gly His Leu Gly Val |
| | Val Leu Tyr Glu Tyr Leu Gly Glu Glu Tyr Pro Glu Val Leu Gly Ser |
| | Ile Leu Gly Ala Leu Lys Ala Ile Val Asn Val Ile Gly Met His Lys |
| | Met Thr Pro Pro Ile Lys Asp Leu Leu Pro Arg Leu Thr Pro Ile |
| | Leu Lys Asn Arg His Glu Lys Val Gln Glu Asn Cys Ile Asp Leu |
| | Val Gly Arg Ile Ala Asp Arg Gly Ala Glu Tyr Val Ser Ala Arg |
| | Glu Trp Met Arg Ile Cys Phe Glu Leu Leu Glu Leu Leu Lys Ala |
| | His Lys Lys Ala Ile Arg Arg Ala Thr Val Asn Thr Phe Gly Tyr |
| | Ile Ala Lys Ala Ile Gly Pro His Asp Val Leu Ala Thr Leu Leu |
| | Asn Asn Leu Lys Val Gln Glu Arg Gln Asn Arg Val Cys Thr Thr |
| | Val Ala Ile Ala Ile Val Ala Glu Thr Cys Ser Pro Phe Thr Val |
| | Leu Pro Ala Leu Met Asn Glu Tyr Arg Val Pro Glu Leu Asn Val |
| | Gln Asn Gly Val Leu Lys Ser Leu Ser Phe Leu Phe Glu Tyr Ile |
| | Gly Glu Met Gly Lys Asp Tyr Ile Tyr Ala Val Thr Pro Leu Leu |
| | Glu Asp Ala Leu Met Asp Arg Asp Leu Val His Arg Gln Thr Ala |
| | Ser Ala Val Val Gln His Met Ser Leu Gly Val Tyr Gly Phe Gly |
| | Cys Glu Asp Ser Leu Asn His Leu Leu Asn Tyr Val Trp Pro Asn |
| | Val Phe Glu Thr Ser Pro His Val Ile Gln Ala Val Met Gly Ala |
| | Leu Glu Gly Leu Arg Val Ala Ile Gly Pro Cys Arg Met Leu Gln |
| | Tyr Cys Leu Gln Gly Leu Phe His Pro Ala Arg Lys Val Arg Asp |
| | Val Tyr Trp Lys Ile Tyr Asn Ser Ile Tyr Ile Gly Ser Gln Asp |
| | Ala Leu Ile Ala His Tyr Pro Arg Ile Tyr Asn Asp Asp Lys Asn |
| | Thr Tyr Ile Arg Tyr Glu Leu Asp Tyr Ile Leu |
| 2 | MAKHHPDLIF CRKQAGVAIG RLCEKCDGKC VICDSYVRPC |
| | TLVRICDECN YGSYQGRCVI CGGPGVSDAY YCKECTIQEK |
| | DRDGCPKIVN LGSSKTDLFY ERKKYGFKKR |
| 3 | actctcttccgcatcgctgtctgcgagggccagctgttggggtgagtactccctctcaaaagcgggcatgactt |
| | ctgcgctaagattgtcagtaccaaaaacgaggaggatttgatattcacctggcccgcggtgatgccatgagg |
| | gtggccgcgtccatctggtcagaaaagacaatctattgagtcaagctagcacgtctagggcgcagtagtcc |
| | agggtaccttgatgatgtcatactaatcctgtcccattattccacagctcgcggttgaggacaaactcttcgcgg |
| | tctttccagtactcttggatcggaaacccgtcggcctccgaacg |
| 4 | ACTCTCTTCCGCATCGCTGT |
| 5 | CCGACGGGTTTCCGATCCAA |
| 6 | CTGTTGGGCTCGCGGTTG |
| 7 | TGGCATCAGATTGCAAAGAC |
| 8 | ACGCCGGGTGATGTATCTAT |
| 9 | CGAAACGCACCCGTCAGACG |
| 10 | ATATGCCAAACCAGCTCCTAC |
| 11 | AGAACTCCACAAACCCATCCCAGC |
| 12 | AAGGACAAAACGGGACTGG |
| 13 | AAAGCCAATGGGCAGGT |
| 14 | TCCACAAACCCATCTTGGAAGGCC |
| 15 | CCACCTTCTAGGTCCTCTACAT |
| 16 | GACAAAGGAGGCCGTGAGGA |
| 17 | GTTTGTTACGCCGTCGCTGAAA |
| 18 | TCAGGCATGCTTCGGAAACTGGA |
| 19 | GCCCCGGGGTGAATAATAATTGGTTTACT |
| 20 | TTTCTAGGATGGGTTTGTGGAGTT |
| 21 | CCTGATGCCACCTTCTAGGTCCTCTAC |
| 22 | GCCAAGGACACAAAGCCAAT |
| 23 | CTGGAGACCTTACGACGGGTTGGG |
| 24 | AAGGCCGTCTCGTGGTT |
| 25 | ATGGCGAAGATCGCCAAGACTCACGAAGATATTGAAGCACAGATTCGAGAAATT |
| | CAAGGCAAGAAGGCAGCTCTTGATGAAGCTCAAGGAGTGGGCCTCGATTCTACA |
| | GGTTATTATGACCAGGAAATTTATGGTGGAAGTGACAGCAGATTTGCTGGATACG |
| | TGACATCAATTGCTGCAACTGAACTTGAAGATGATGACGATGACTATTCATCATC |
| | TACGAGTTTGCTTGGTCAGAAGAAGCCAGGATATCATGCCCCTGTGGCATTGCT |
| | TAATGATATACCACAGTCAACAGAACAGTATGATCCATTTGCTGAGCACAGACCT |
| | CCAAAGATTGCAGACCGGGAAGATGAATACAAAAAGCATAGGCGGACCATGATA |
| | ATTTCCCCAGAGCGTCTTGATCCTTTTGCAGATGGAGGGAAAACCCCTGATCCTA |
| | AAATGAATGCTAGGACTTACATGGATGTAATGCGAGAACAACACTTGACTAAAG |
| | AAGAACGAGAAATTAGGCAACAGCTAGCAGAAAAAGCTAAAGCTGGAGAACTAA |
| | AAGTCGTCAATGGAGCAGCAGCGTCCCAGCCTCCATCAAAACGAAAACGGCGTT |
| | GGGATCAAACAGCTGATCAGACTCCTGGTGCCACTCCCAAAAAACTATCAAGTT |
| | GGGATCAGGCAGAGACCCCTGGGCATACTCCTTCCTTAAGATGGGATGAGACAC |
| | CAGGTCGTGCAAAGGGAAGCGAGACTCCTGGAGCAACCCCAGGCTCAAAAATAT |
| | GGGATCCTACACCTAGCCACACACCAGCGGGAGCTGCTACTCCTGGACGAGGT |
| | GATACACCAGGCCATGCGACACCAGGCCATGGAGGCGCAACTTCCAGTGCTCG |
| | TAAAAACAGATGGGATGAAACCCCCAAAACAGAGAGAGATACTCCTGGGCATGG |
| | AAGTGGATGGGCTGAGACTCCTCGAACAGATCGAGGTGGAGATTCTATTGGTGA |
| | AACACCGACTCCTGGAGCCAGTAAAAGAAAATCACGGTGGGATGAAACACCAGC |
| | TAGTCAGATGGGTGGAAGCACTCCAGTTCTGACCCCTGGAAAGACACCAATTGG |
| | CACACCAGCCATGAACATGGCTACCCCTACTCCAGGTCACATAATGAGTATGACT |
| | CCTGAACAGCTTCAGGCTTGGCGGTGGGAAAGAGAAATTGATGAGAGAAATCG |
| | CCCACTTTCTGATGAGGAATTAGATGCTATGTTCCCAGAAGGATATAAGGTACTT |
| | CCTCCTCCAGCTGGTTATGTTCCTATTCGAACTCCAGCTCGAAAGCTGACAGCTA |
| | CTCCAACACCTTTGGGTGGTATGACTGGTTTCCACATGCAAACTGAAGATCGAAC |
| | TATGAAAAGTGTTAATGACCAGCCATCTGGAAATCTTCCATTTTTAAAACCTGATG |
| | ATATTCAATACTTTGATAAACTATTGGTTGATGTTGATGAATCAACACTTAGTCCA |
| | GAAGAGCAAAAAGAGAGAAAAATAATGAAGTTGCTTTTAAAAATTAAGAATGGAA |
| | CACCACCAATGAGAAAGGCTGCATTGCGTCAGATTACTGATAAAGCTCGTGAATT |
| | TGGAGCTGGTCCTTTGTTTAATCAGATTCTTCCTCTGCTGATGTCTCCTACACTTG |
| | AGGATCAAGAGCGTCATTTACTTGTGAAAGTTATTGATAGGATACTGTACAAACTT |
| | GATGACTTAGTTCGTCCATATGTGCATAAGATCCTCGTGGTCATTGAACCGCTAT |
| | TGATTGATGAAGATTACTATGCTAGAGTGGAAGGCCGAGAGATCATTTCTAATTT |
| | GGCAAAGGCTGCTGGTCTGGCTACTATGATCTCTACCATGAGACCTGATATAGAT |
| | AACATGGATGAGTATGTCCGTAACACAACAGCTAGAGCTTTTGCTGTTGTAGCCT |
| | CTGCCCTGGGCATTCCTTCTTTATTGCCCTTCTTAAAAGCTGTGTGCAAAAGCAA |
| | GAAGTCCTGGCAAGCGAGACACACTGGTATTAAGATTGTACAACAGATAGCTATT |
| | CTTATGGGCTGTGCCATCTTGCCACATCTTAGAAGTTTAGTTGAAATCATTGAAC |
| | ATGGTCTTGTGGATGAGCAGCAGAAAGTTCGGACCATCAGTGCTTTGGCCATTG |
| | CTGCCTTGGCTGAAGCAGCAACTCCTTATGGTATCGAATCTTTTGATTCTGTGTT |
| | AAAGCCTTTATGGAAGGGTATCCGCCAACACAGAGGAAAGGGTTTGGCTGCTTT |
| | CTTGAAGGCTATTGGGTATCTTATTCCTCTTATGGATGCAGAATATGCCAACTACT |
| | ATACTAGAGAAGTGATGTTAATCCTTATTCGAGAATTCCAGTCTCCTGATGAGGA |
| | AATGAAAAAAATTGTGCTGAAGGTGGTAAAACAGTGTTGTGGGACAGATGGTGTA |
| | GAAGCAAACTACATTAAAACAGAGATTCTTCCTCCCTTTTTTAAACACTTCTGGCA |
| | GCACAGGATGGCTTTGGATAGAAGAAATTACCGACAGTTAGTTGATACTACTGTG |
| | GAGTTGGCAAACAAAGTAGGTGCAGCAGAAATTATATCCAGGATTGTGGATGAT |
| | CTGAAAGATGAAGCCGAACAGTACAGAAAAATGGTGATGGAGACAATTGAGAAA |
| | ATTATGGGTAATTTGGGAGCAGCAGATATTGATCATAAACTTGAAGAACAACTGA |
| | TTGATGGTATTCTTTATGCTTTCCAAGAACAGACTACAGAGGACTCAGTAATGTT |
| | GAACGGCTTTGGCACAGTGGTTAATGCTCTTGGCAAACGAGTCAAACCATACTT |
| | GCCTCAGATCTGTGGTACAGTTTTGTGGCGTTTAAATAACAAATCTGCTAAAGTT |
| | AGGCAACAGGCAGCTGACTTGATTTCTCGAACTGCTGTTGTCATGAAGACTTGTC |
| | AAGAGGAAAAATTGATGGGACACTTGGGTGTTGTATTGTATGAGTATTTGGGTGA |
| | AGAGTACCCTGAAGTATTGGGCAGCATTCTTGGAGCACTGAAGGCCATTGTAAA |
| | TGTCATAGGTATGCATAAGATGACTCCACCAATTAAAGATCTGCTGCCTAGACTA |
| | CCCCCATCTTAAAGAACAGACATGAAAAAGTACAAGAGAATTGTATTGATCTTGTT |
| | GGTCGTATTGCTGACAGGGGAGCTGAATATGTATCTGCAAGAGAGTGGATGAGG |
| | ATTTGCTTTGAGCTTTTAGAGCTCTTAAAAGCCCACAAAAAGGCTATTCGTAGAG |
| | CCACAGTCAACACATTTGGTTATATTGCAAAGGCCATTGGCCCTCATGATGTATT |
| | GGCTACACTTCTGAACAACCTCAAAGTTCAAGAAAGGCAGAACAGAGTTTGTACC |
| | ACTGTAGCAATAGCTATTGTTGCAGAAACATGTTCACCCTTTACAGTACTCCCTG |
| | CCTTAATGAATGAATACAGAGTTCCTGAACTGAATGTTCAAAATGGAGTGTTAAAA |
| | TCGCTTTCCTTCTTGTTTGAATATATTGGTGAAATGGGAAAAGACTACATTTATGC |
| | CGTAACACCGTTACTTGAAGATGCTTTAATGGATAGAGACCTTGTACACAGACAG |
| | ACGGCTAGTGCAGTGGTACAGCACATGTCACTTGGGGTTTATGGATTTGGTTGT |
| | GAAGATTCGCTGAATCACTTGTTGAACTATGTATGGCCCAATGTATTTGAGACAT |
| | CTCCTCATGTAATTCAGGCAGTTATGGGAGCCCTAGAGGGCCTGAGAGTTGCTA |
| | TTGGACCATGTAGAATGTTGCAATATTGTTTACAGGGTCTGTTTCACCCAGCCCG |
| | GAAAGTCAGAGATGTATATTGGAAAATTTACAACTCCATCTACATTGGTTCCCAG |
| | GACGCTCTCATAGCACATTACCCAAGAATCTACAACGATGATAAGAACACCTATA |
| | TTCGTTATGAACTTGACTATATCTTATAA |
| 26 | ATGGCTAAACATCATCCTGATTTGATCTTTTGCCGCAAGCAGGCTGGTGTTGCCA |
| | TCGGAAGACTGTGTGAAAAATGTGATGGCAAGTGTGTGATTTGTGACTCCTATGT |
| | GCGTCCCTGCACTCTGGTGCGCATATGTGATGAGTGTAACTATGGATCTTACCA |
| | GGGGCGCTGTGTGATCTGTGGAGGACCTGGGGTCTCTGATGCCTATTATTGTAA |
| | GGAGTGCACCATCCAGGAGAAGGACAGAGATGGCTGCCCAAAGATTGTCAATCT |
| | GGGGAGCTCTAAGACAGACCTCTTCTATGAACGCAAAAAATACGGCTTCAAGAA |
| | GAGGTGA |

1-81. (canceled)

82. A method of treating a subject having a neoplastic disorder, comprising administering a splicing modulator to the subject lacking a PHF5A mutation, or administering an alternative treatment that does not target the spliceosome to the subject having a PHF5A mutation.

83. The method of claim 82, further comprising detecting the presence or absence of a PHF5A mutation in the subject.

84. The method of claim 82, further comprising detecting the presence or absence of a PHF5A mutation in the subject administered the splicing modulator; and administering a further dose of the splicing modulator to the subject if a PHF5A mutation is absent.

85. The method of claim 82, further comprising obtaining a biological sample from the subject, wherein the presence or absence of a PHF5A mutation is detected in the sample.

86. The method of claim 85, wherein the sample comprises a tumor sample, blood, or a blood fraction.

87. The method of claim 83, wherein detecting the presence or absence of a PHF5A mutation comprises:
a) obtaining a tumor sample from the subject;
b) contacting the sample with a splicing modulator;
c) measuring the growth and/or volume of the sample contacted with the splicing modulator; and
d) comparing the growth and/or volume of the sample to a control tumor sample of known PHF5A mutation status,
wherein a change or lack of change in the growth and/or volume of the sample as compared to the control tumor sample indicates the presence or absence of a PHF5A mutation.

88. The method of claim 82, wherein the splicing modulator comprises a SF3b complex modulator, a SF3B1 complex modulator, and/or a PHF5A modulator.

89. The method of claim 82, wherein the splicing modulator comprises a pladienolide or pladienolide derivative, a herboxidiene or herboxidiene derivative, a spliceostatin or spliceostatin derivative, a sudemycin or sudemycin derivative, or a combination thereof.

90. The method of claim 89, wherein the pladienolide or pladienolide derivative comprises E7107, pladienolide B, or pladienolide D; wherein the herboxidiene or herboxidiene derivative comprises 6-nor herboxidiene; wherein the spliceostatin or spliceostatin derivative comprises FR901464 or spliceostatin A; and/or wherein the sudemycin or sudemycin derivative comprises sudemycin D6.

91. The method of claim 82, wherein the PHF5A mutation is located in or near the PHF5A-SF3B1 interface.

92. The method of claim 82, wherein the PHF5A mutation comprises a Y36 mutation in PHF5A.

93. The method of claim 92, wherein the Y36 mutation comprises a Y36C, Y36A, Y36C, Y36S, Y36F, Y36W, Y36E, or Y36R mutation in PHF5A.

94. The method of claim 82, wherein the alternative treatment that does not target the spliceosome comprises a cytotoxic agent, a cytostatic agent, and/or a proteasome inhibitor.

95. The method of claim 94, wherein the proteasome inhibitor comprises bortezomib.

96. The method of claim 82, further comprising detecting the presence or absence of a SF3B1 mutation in the subject.

97. The method of claim 96, further comprising administering a pladienolide or pladienolide derivative to the subject lacking a SF3B1 mutation and a PHF5A mutation.

98. The method of claim 97, wherein the pladienolide or pladienolide derivative comprises E7107.

99. The method of claim 96, wherein the SF3B1 mutation comprises a E622D, E622K, E622Q, E622V, Y623C, Y623H, Y623S, R625C, R625G, R625H, R625L, R625P, R625S, R1074H, N626D, N626H, N626I, N626S, N626Y, H662D, H662L, H662Q, H662R, H662Y, T663I, T663P, K666E, K666M, K666N, K666Q, K666R, K666S, K666T, K700E, V701A, V701F, V701I, I704F, I704N, I704S, I704V, G740E, G740K, G740R, G740V, K741N, K741Q, K741T, G742D, D781E, D781G, and/or D781N mutation in SF3B1.

100. The method of claim 99, wherein the SF3B1 mutation further comprises a R1074H mutation in SF3B1.

101. The method of claim 82, wherein the subject has a cancer comprising a mutation at one or more of positions K1071, R1074, and V1078 in SF3B1.

102. The method of claim 82, wherein the subject has a cancer comprising a K1071E, R1074H, V1078A, and/or V1078I mutation in SF3B1.

103. The method of claim 82, wherein the subject has a cancer comprising a Y36C mutation in PHF5A, and a K1071E, R1074H, V1078A, and/or V1078I mutation in SF3B1.

104. The method of claim 82, wherein the neoplastic disorder is a hematological malignancy, a solid tumor, or a soft tissue sarcoma.

105. The method of claim 104, wherein the hematological malignancy is myelodysplastic syndrome, chronic lymphocytic leukemia, chronic myelomonocytic leukemia, or acute myeloid leukemia.

106. The method of claim 96, wherein detecting the presence or absence of a PHF5A mutation comprises comparing PHF5A in the subject to a wild-type PHF5A nucleic acid or protein sequence; and/or wherein detecting the presence or absence of a mutation in SF3B1 comprises comparing SF3B1 in the subject to a wild-type SF3B1 nucleic acid or protein sequence.

107. The method of claim 96, wherein detecting the presence or absence of a PHF5A mutation comprises sequencing the gene encoding PHF5A in the subject; and/or wherein detecting the presence or absence of a SF3B1 mutation comprises sequencing the gene encoding SF3B1 in the subject.

108. The method of claim 107, wherein sequencing comprises PCR amplification, real time-PCR, in situ PCR, Sanger sequencing, whole exome sequencing, single nucleotide polymorphism analysis, deep sequencing, targeted gene sequencing, or a combination thereof.

109. A method of treating a subject having a neoplastic disorder, comprising:
a) detecting the presence or absence of a PHF5A mutation in the subject; and
b) administering a splicing modulator to the subject lacking a PHF5A mutation, or administering an alternative treatment that does not target the spliceosome to the subject having a PHF5A mutation.

110. The method of claim 109, further comprising obtaining a biological sample from the subject, wherein the presence or absence of a PHF5A mutation is detected in the sample.

111. The method of claim 110, wherein the sample comprises a tumor sample, blood, or a blood fraction.

112. A method of identifying a subject having a neoplastic disorder that is resistant or responsive to a splicing modulator, comprising:
a) detecting the presence or absence of a PHF5A mutation in a sample from the subject; and
b) identifying the subject as having a treatment-resistant neoplastic disorder if a PHF5A mutation is detected in the sample; or identifying the subject as having a treatment-responsive neoplastic disorder if a PHF5A mutation is not detected in the sample.

113. A method of monitoring treatment efficacy in a subject having a neoplastic disorder, comprising:
a) administering a splicing modulator to the subject;
b) detecting the presence or absence of a PHF5A mutation in the subject administered the splicing modulator;
c) administering a further dose of the splicing modulator to the subject if a PHF5A mutation is absent; and
d) continuing to repeat steps a)-c) until a PHF5A mutation is detected.

114. A kit comprising:
a) a reagent capable of detecting a PHF5A mutation; and
b) instructions for use of the reagent to detect a PHF5A mutation.

115. A method of treating a subject having a neoplastic disorder, comprising administering a splicing modulator to the subject lacking a SF3B1 mutation, or administering an alternative treatment that does not target the spliceosome to the subject having a SF3B1 mutation.

116. The method of claim 115, further comprising detecting the presence or absence of a SF3B1 mutation in the subject.
